(19) United States
(12) Patent Application Publication
(10) Pub. No.: US 2019/0133963 A1
(43) Pub. Date: May 9, 2019

(54) POLYMERIC NANOPARTICLES

(71) Applicant: NANOPROTEAGEN, Grand Cayman (KY)

(72) Inventors: SURENDER KHARBANDA, NATICK, MA (US); HARPAL SINGH, NEW DELHI (IN); DIKSHI GUPTA, GURGAON (IN)

(73) Assignee: NANOPROTEAGEN, Grand Cayman (KY)

(21) Appl. No.: 15/773,392

(22) PCT Filed: Nov. 3, 2016
(86) PCT No.: PCT/US2016/060276

§ 371 Date: May 3, 2018

Related U.S. Application Data
(60) Provisional application No. 62/250,137, filed on Nov. 3, 2015.
(60) Provisional application No. 62/358,373, filed on Jul. 5, 2016.

(51) Int. Cl. A61K 9/51; A61K 38/17; A61K 31/337; A61K 31/7068; A61K 47/69; A61P 35/00; C08G 81/00

(52) U.S. Cl. CPC A61K 9/5153; A61K 38/1761; A61K 38/1735; C08G 81/00; A61K 31/7068; A61K 47/6937; A61P 35/00; A61K 31/337

(57) ABSTRACT

The present invention relates to polymeric nanoparticles comprising a pharmaceutical combination, pharmaceutical compositions comprising the same, and methods for treating certain diseases comprising administering these polymeric nanoparticles to a subject in need thereof.

### RELATED APPLICATIONS

This application claims priority to U.S. Provisional Application No. 62/250,137, filed Nov. 3, 2015, and U.S. Provisional Application No. 62/358,373, filed Jul. 5, 2016, the contents of which are incorporated herein in their entirety.

### FIELD OF INVENTION

The present invention relates to the field of nanotechnology, in particular, to the use of biodegradable polymeric nanoparticles for the delivery of therapeutic agents.

### BACKGROUND

Cancer is one of the most devastating diseases and it involves various genetic alterations and cellular abnormalities. This complexity and heterogeneity promotes the aggressive growth of cancer cells leading to significant morbidity and mortality in patients (Das, M. et al. (2009) Ligand-based targeted therapy for cancer tissue. Expert Opin. Drug Deliv. 6, 285-304; Mohanty, C. et al. (2011) Receptor mediated tumor targeting: an emerging approach for cancer therapy. Curr. Drug Deliv. 8, 45-58). Breast cancer is one of the most commonly diagnosed cancers and is the second leading cause of death among women. Paclitaxel (“PTX”) is a widely used chemotherapy drug in the treatment of breast cancer and other solid tumors (Holmes F., et al. Phase II trial of taxol, an active drug in the treatment of metastatic breast cancer. J. Natl. Cancer Inst. 1991, 83(24):1797-1805; Brown T., et al. A phase I trial of taxol given by a 6-hour intravenous infusion. J. Clin. Oncol. 1991, 9(7):1261-1267; McGuire W., et al.: Taxol: a unique antineoplastic agent with significant activity in advanced ovarian epithelial neoplasms. Ann. Intern. Med. 1989, 111(4):273-279). It inhibits microtubule disassembly when it binds to assembled tubulins, making the microtubules locked in polymerized state (Jordan M., Kamath K.: How do microtubule-targeted drugs work? An overview. Curr. Cancer Drug Targets 2007, 7(8):730-742) leading to cell cycle arrest (Fuchs D., Johnson R.: Cytologic evidence that taxol, an antineoplastic agent from Taxus brevifolia, acts as a mitotic spindle poison. Cancer Treat. Rep. 1978, 62(8):1219-1222; Schiff P., Horwitz S B: Taxol stabilizes microtubules in mouse fibroblast cells. Proc. Natl. Acad. Sci. USA 1980, 77(3):1561-1565; Schiff P., Horwitz S.: Taxol assembles tubulin in the absence of exogenous guanosine 5′-triphosphate or microtubule-associated proteins. Biochemistry 1981, 20(11):3247-3252; Schiff P., et al.: Promotion of microtubule assembly in vitro by taxol. Nature 1979, 277(5698):665-667). Paclitaxel also inhibits the anti-apoptotic protein BCL-2, and induces apoptosis in cancer cells (Haldar S., et al.: Inactivation of BCL-2 by phosphorylation. Proc. Natl. Acad. Sci. USA 1995, 92(10):4507-4511). Paclitaxel is a highly effective anti-neoplastic agent but its high dose and repeated treatment may result in high cytotoxicity and drug resistance which limits the prolonged use in patients (Brown T., et al. J. Clin. Oncol. 1991, 9(7):1261-1267; Wiernik P., et al.: Phase I clinical and pharmacokinetic study of taxol. Cancer Res 1987, 47(9):2486-2493; Wiernik P., et al.: Phase I trial of taxol given as a 24-hour infusion every 21 days: responses observed in metastatic melanoma. J. Clin. Oncol. 1987, 5(8):1232-1239).

PTX was initially developed for breast cancer treatment in a solvent-based formulation consisting of polyoxyethylated castor oil, which was associated with clinically significant hypersensitivity reactions. Nab-paclitaxel (Abraxane) is a second generation formulation in which PTX is encapsulated in solvent-free albumin NPs (Yardley D A, et al. (2013) Randomized phase II, double-blind, placebo-controlled study of exemestane with or without entinostat in postmenopausal women with locally recurrent or metastatic estrogen receptor-positive breast cancer progressing on treatment with a nonsteroidal aromatase inhibitor. J. Clin Oncol 31(17):2128-2135). Nab-paclitaxel can be delivered at higher doses than PTX by, in part, circumventing the hypersensitivity reactions (Ibrahim N K, et al. (2005) Multicenter phase II trial of ABI-007, an albumin-bound paclitaxel, in women with metastatic breast cancer. J. Clin. Oncol 23(25):6019-6026; Yardley D A et al. (2013), J. Clin. Oncol 31(17):2128-2135). In addition, nab-paclitaxel was found to be more effective than PTX in the treatment of patients with breast cancer (Gradishar W J, et al. (2005) Phase III trial of nanoparticle albumin-bound paclitaxel compared with polyethylated castor oil-based paclitaxel in women with breast cancer. J. Clin. Oncol 23(31):7794-7803; Blum J L, et al. (2007) Phase II study of weekly albumin-bound paclitaxel for patients with metastatic breast cancer heavily pretreated with taxanes. Clin Breast Cancer 7(11):850-856; 30; Gradishar W J, et al. (2012) Phase II trial of nab-paclitaxel compared with docetaxel as first-line chemotherapy in patients with metastatic breast cancer: final analysis of overall survival. Clin Breast Cancer 12(5):313-321). Thus, the approval of nab-paclitaxel for the treatment of breast, as well as NSCLC and pancreatic cancers has supported the effectiveness of delivering PTX in a NP formulation. However, the progression-free survival for PTX and nab-paclitaxel as first-line treatment of locally recurrent or metastatic breast cancer is 11 and 9.3 months, respectively (Rugo H S, et al. (2015) Randomized Phase III Trial of Paclitaxel Once Per Week Compared With Nanoparticle Albumin-Bound Nab-Paclitaxel Once Per Week or Ixabepilone With Bevacizumab As First-Line Chemotherapy for Locally Recurrent or Metastatic Breast Cancer: CALGB 40502/NCCTG N063H (Alliance). J. Clin Oncol. 33(21):2361-2369), emphasizing the need for more effective therapies that circumvent the development of resistance.

PTX induces a multidrug resistance (MDR) phenotype in large part by overexpression of the ABC family of transporters (Barbuti A M & Chen Z S (2015) Paclitaxel Through the Ages of Anticancer Therapy: Exploring Its Role in Chemoresistance and Radiation Therapy. Cancers (Basel) 7(4):2360-2371; Zhao Y, Mu X, & Du G (2015) Microtubule-stabilizing agents: New drug discovery and cancer therapy. Pharmacol Ther.). Among the subclasses of ABC transporters, overexpression of Pgp1 (ABCB1, MDR1) represents a major mechanism of PTX resistance (Barbuti A M & Chen Z S (2015) Cancers (Basel) 7(4):2360-2371; Zhao Y, Mu X, & Du G (2015) Pharmacol Ther.). However, despite years of research, there has been limited progress in the development of P-gp inhibitors that increase the effectiveness of PTX in the absence of unacceptable toxicity (Gottesman M M, Fojo T, & Bates S E (2002) Multidrug resistance in cancer: role of ATP-dependent transporters. Nat Rev Cancer 2(1):48-58).

Combination therapy has been adopted in clinics to address the problems associated with Paclitaxel cancer treatment. By combining paclitaxel with one or more agents like cisplatin, 5-fluoro uracil (5-FU), or gemcitabine, chemotherapy resistance and side-effects associated with high doses can be overcome by countering different biological signaling pathways synergistically, enabling a low dosage of each compound. Applying multiple drugs with different molecular targets can raise the genetic barriers that need to be overcome for cancer cell mutations, thereby delaying the cancer adaptation process. It has also been demonstrated that multiple drugs targeting the same cellular pathways could function synergistically for higher therapeutic efficacy and higher target selectivity (Lehar J., et al. Synergistic drug combinations tend to improve therapeutically relevant selectivity. Nat. Biotechnol. 27(7), 659-666 (2009)). Nanotechnology can make significant advances in cancer therapy by offering a smart drug delivery system.

However, conventional combination therapy has not proved successful for cancer due to low bioavailability and optimal biodistribution of drugs at the target site. Wang et al. showed the co-administration of paclitaxel (PTX) and doxorubicin using micelles of stearate-grafted chitosan oligosaccharide (CSO-SA) (Zhao, M. et al. Coadministration of glycolipid-like micelles loading cytotoxic drug with different action site for efficient cancer chemotherapy. Nanotechnology 2009, 20, 055102). Another study employed nanoparticles of poly(D,L-lactide-co-glycolide acid) (PLGA) for simultaneous delivery of vincristine (VCR) and verapamil (VRP) (Song, X. et al. PLGA nanoparticles simultaneously loaded with vincristine sulfate and verapamil hydrochloride: Systematic study of particle size and drug entrapment efficiency. Int. J. Pharm. 2008, 35, 320-329). Liposomal delivery formulation for quercetin and VCR was also developed (Wong, M.-Y.; Chiu, G. N. C. Simultaneous liposomal delivery of quercetin and vincristine for enhanced estrogen-receptor-negative breast cancer treatment. Anti-Cancer Drugs 2010, 21, 401-410). However these formulations still showed high toxicity due to combination of chemotherapeutic drugs.

Biomolecules have been adopted in research along with chemo drugs for lower toxicity and better therapeutic effectiveness. Kwon et al. reported that poly(ethylene glycol)-block-poly(D,L-lactic acid) (PEG-b-PLA) micelles can deliver multiple drugs including combinations of PTX/17-allylamino-17-demethoxygeldanamycin (17-AAG) (Kwon, G. S. et al. Multi-drug loaded polymeric micelles for simultaneous delivery of poorly soluble anticancer drugs. J. Controlled Release 2009, 140, 294-300). PTX with BCL-2 targeted siRNA using cationic core shell nanoparticles have been reported for breast cancer treatment. Sugahara et al. showed that co-administration of iRGD (a tumor-penetrating peptide) with different types of cancer drugs are effective in inhibiting tumor growth and tumor accumulation (Sugahara K N, et al. Co-administration of a Tumor-Penetrating Peptide Enhances the Efficacy of Cancer Drugs. Science. 2010; 328:1031-1035). In such combinations, the effective cytotoxic doses of chemotherapeutic drugs are dramatically reduced with a concomitant decrease in adverse events, so this strategy represents a superior approach to the use of single chemo-drug with biomolecule (Wang S. Z., et al. TRAIL and Doxorubicin Combination Induces Proapoptotic and Antiangiogenic Effects in Soft Tissue Sarcoma in vivo. Clin. Cancer Res. 2010; 16:2591-2604; Hossain M A, et al. Aspirin enhances doxorubicin-induced apoptosis and reduces tumor growth in human hepatocellular carcinoma cells in vitro and in vivo. Int. J. Oncol. 2012; 40:1636-1642; Jin C., et al. Combination chemotherapy of doxorubicin and paclitaxel for hepatocellular carcinoma in vitro and in vivo. J. Cancer Res. Clin. 2010; 136:267-274).

Further, molecularly targeted therapy has emerged as a promising approach to overcome the lack of specificity of conventional chemotherapeutic agents in the treatment of cancer. Synthetic peptide drugs in cancer therapy show high specificity, stability and ease of synthesis compared to conventional proteins. However, the delivery of these anti-cancer peptides to the target site poses huge problems due to factors like enzymatic degradation, immunogenicity, and a short life span in the blood. Targeted delivery of anticancer drugs would be more effective if the delivery system was able to reach the desired tumor tissues through the penetration of barriers in the body with minimal loss of their volume or activity in the blood circulation and selectively kill tumor cells. This would improve patient survival and quality of life by increasing the intracellular concentration of drugs and reducing dose-limiting toxicities simultaneously. One of the strategies for delivery of peptide drugs involves conjugating peptides with cell penetrating peptides (CPP) for direct delivery of the drug into cytosol. However, conjugation with CPP increases the cost and decreases the efficacy and stability of peptide drugs, and can in some instances increase toxicity. Some peptidic therapeutic agents like NuBCP-9 and Bax-BH3 show selective binding to cancerous cells and initiate apoptosis. Unfortunately, free drug formulations of peptidic therapeutic agents require the use of large amounts and frequent administration of the peptide, thereby increasing the cost and inconvenience of therapy.

There is a pressing need for a delivery system that can effectively deliver therapeutic agents, such as therapeutic peptides, alone, or in combination with other therapeutic agents such as chemotherapeutic agents, into cancerous cells. Furthermore, there is a need for a delivery system capable of treating cancers resistant to traditional chemotherapeutics, e.g., paclitaxel or nab-paclitaxel.

### SUMMARY

In an aspect, provided herein is a composition comprising

a) polymeric nanoparticles comprising a poly(lactic acid)-poly(ethylene glycol)-poly(propylene glycol)-poly(ethylene glycol) (PLA-PEG-PPG-PEG) tetra block copolymer;

b) one or more chemotherapeutic agents or anti-cancer targeting agents; and

c) a peptide comprising NuBCP-9 (SEQ ID NO: 1) or a peptide comprising MUC1 (SEQ ID NO: 2).

In an embodiment of the composition, the composition comprises a peptide comprising NuBCP-9 (SEQ ID NO: 1).

In another embodiment of the composition, the composition comprises a peptide comprising MUC1 (SEQ ID NO: 2).

In an embodiment of the composition, the molecular weight of PLA is between about 2,000 and about 80,000 daltons.

In an embodiment of the composition, the PLA-PEG-PPG-PEG tetra block copolymer is formed from chemical conjugation of PEG-PPG-PEG tri-block copolymer with PLA, and the PEG-PPG-PEG tri-block copolymer can be of different molecular weights.

In an embodiment of the composition, the polymeric nanoparticles are loaded with

a) a chemotherapeutic agent or a targeted anti-cancer agent; and

b) a peptide comprising NuBCP-9 (SEQ ID NO: 1) or a peptide comprising MUC1 (SEQ ID NO: 2).

In a further embodiment of the composition, the polymeric nanoparticles are loaded with

a) a chemotherapeutic agent or a targeted anti-cancer agent; and

b) a peptide comprising NuBCP-9 (SEQ ID NO: 1).

In another further embodiment of the composition, the polymeric nanoparticles are loaded with

a) a chemotherapeutic agent or a targeted anti-cancer agent; and

b) a peptide comprising MUC1 (SEQ ID NO: 2).

In a further embodiment of the composition, the chemotherapeutic agent is paclitaxel.

In yet a further embodiment of the composition, the polymeric nanoparticles are loaded with paclitaxel and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the composition, the chemotherapeutic agent is gemcitabine. In a further embodiment of the composition, the polymeric nanoparticles are loaded with gemcitabine and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the composition, the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

In another aspect, provided herein is a pharmaceutical composition comprising

a) polymeric nanoparticles comprising a poly(lactic acid)-poly(ethylene glycol)-poly(propylene glycol)-poly(ethylene glycol) (PLA-PEG-PPG-PEG) tetra block copolymer;

b) one or more therapeutic agents; and

c) a peptide comprising NuBCP-9 (SEQ ID NO: 1) or a peptide comprising MUC1 (SEQ ID NO: 2),

for use in treating a disease selected from the group consisting of cancer, an autoimmune disease, an inflammatory disease, a metabolic disorder, a developmental disorder, a cardiovascular disease, liver disease, an intestinal disease, an infectious disease, an endocrine disease and a neurological disorder.

In an embodiment of the pharmaceutical composition, the composition comprises a peptide comprising NuBCP-9 (SEQ ID NO: 1).

In another embodiment of the pharmaceutical composition, the composition comprises a peptide comprising MUC1 (SEQ ID NO: 2).

In an embodiment of any of the compositions provided herein, the polymeric nanoparticles consist essentially of poly(lactic acid)-poly(ethylene glycol)-poly(propylene glycol)-poly(ethylene glycol) (PLA-PEG-PPG-PEG) tetra block copolymer.

In an embodiment of any of the compositions provided herein, the polymeric nanoparticles further comprise a targeting moiety attached to the outside of the polymeric nanoparticles, and the targeting moiety is an antibody, peptide, or aptamer.

In another aspect, provided herein is a polymeric nanoparticle consisting essentially of a PLA-PEG-PPG-PEG tetra block copolymer wherein the polymeric nanoparticle is loaded with paclitaxel and a peptide comprising NuBCP-9 (SEQ ID NO: 1).

In another aspect, provided herein is a polymeric nanoparticle consisting essentially of a PLA-PEG-PPG-PEG tetra block copolymer wherein the polymeric nanoparticle is loaded with paclitaxel and a peptide comprising MUC1 (SEQ ID NO: 2).

In another aspect, provided herein is a method for treating cancer in a subject in need thereof comprising administering to the subject a therapeutically effective amount of a pharmaceutical composition comprising

a) polymeric nanoparticles comprising a PLA-PEG-PPG-PEG tetra block copolymer;

b) a chemotherapeutic agent and/or an anti-cancer targeted agent; and

b) a peptide comprising NuBCP-9 (SEQ ID NO: 1) or a peptide comprising MUC1 (SEQ ID NO: 2).

In an embodiment of the method, the pharmaceutical composition comprises a peptide comprising NuBCP-9 (SEQ ID NO: 1).

In another embodiment of the method, the pharmaceutical composition comprises a peptide comprising MUC1 (SEQ ID NO: 2).

In an embodiment of the method, the chemotherapeutic agent is paclitaxel. In a further embodiment of the method, the polymeric nanoparticles are loaded with paclitaxel and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the method, the chemotherapeutic agent is gemcitabine. In a further embodiment of the method, the polymeric nanoparticles are loaded with gemcitabine and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the method, the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

In an embodiment of the method, the cancer is breast cancer, prostate cancer, non-small cell lung cancer, metastatic colon cancer, pancreatic cancer, or a hematological malignancy.

In an embodiment of the method, the subject is resistant to treatment with paclitaxel or nab-paclitaxel.

In an embodiment of the method, the subject is refractory to treatment with paclitaxel or nab-paclitaxel.

In another embodiment of the method, the subject is in relapse after treatment with paclitaxel or nab-paclitaxel.

In another aspect, provided herein is a method for inhibiting paclitaxel efflux in a cell comprising contacting the cell with an effective amount of polymeric nanoparticles comprising PLA-PEG-PPG-PEG tetra block copolymer.

In an embodiment of the method, the polymeric nanoparticles are loaded with paclitaxel.

In yet another aspect, provided herein is a method for blocking P-glycoprotein expression in a cell comprising contacting the cell with an effective amount of polymeric nanoparticles comprising PLA-PEG-PPG-PEG tetra block copolymer.

In another aspect, provided herein is a method for reversing P-glycoprotein-mediated drug resistance in a cell comprising contacting the cell with an effective amount of polymeric nanoparticles comprising PLA-PEG-PPG-PEG tetra block copolymer.

In an embodiment of any of the methods provided herein, the polymeric nanoparticles consist essentially of PLA-PEG-PPG-PEG tetra block copolymer.

In another aspect, provided herein is a method for causing a cancer cell having resistance against a first chemotherapeutic comprising contacting the cancer cell with polymeric nanoparticles comprising PLA-PEG-PPG-PEG tetra block copolymer, wherein the polymeric nanoparticles are loaded with a second chemotherapeutic, and wherein the resistance of the cancer cell against the first chemotherapeutic is caused by upregulation of P-glycoprotein.

In an embodiment, the polymeric nanoparticles consist essentially of PLA-PEG-PPG-PEG tetra block copolymer.

In an embodiment, the cancer cell is a breast cancer cell.

In an embodiment, the first chemotherapeutic is paclitaxel.

In an embodiment, the second chemotherapeutic is paclitaxel.

In an embodiment, the polymeric nanoparticles are loaded with a peptide comprising NuBCP-9 (SEQ ID NO: 1).

In another embodiment, the polymeric nanoparticles are loaded with a peptide comprising MUC1 (SEQ ID NO: 2).

### BRIEF DESCRIPTION OF THE FIGURES

The following figures form part of the present specification and are included to further illustrate aspects of the present invention. The invention may be better understood by reference to the figures in combination with the detailed description of the specific embodiments presented herein.

FIG. 1 provides the schematic diagram of the polymeric nanoparticles of PLA-PEG-PPG-PEG tetra block copolymer.

FIG. 2 provides FTIR spectra of PLA, PEG-PPG-PEG and PLA-PEG-PPG-PEG nanoparticles.

FIG. 3A shows the Nuclear Magnetic Resonance (NMR) spectra of PLA-PEG-PPG-PEG nanoparticles synthesized from a block copolymer of PEG-PPG-PEG of 1,100 g/mol.

FIG. 3B shows the Nuclear Magnetic Resonance (NMR) spectra of PLA-PEG-PPG-PEG nanoparticles synthesized from a block copolymer of PEG-PPG-PEG of 4,400 g/mol.

FIG. 3C shows the Nuclear Magnetic Resonance (NMR) spectra of PLA-PEG-PPG-PEG nanoparticles synthesized from a block copolymer of PEG-PPG-PEG of 8,400 g/mol.

FIG. 4A and FIG. 4B show Transmission Electron Micrograph (TEM) images of PLA-PEG-PPG-PEG polymeric nanoparticles.

FIG. 5A, FIG. 5B, and FIG. 5C show the cellular internalisation of PLA-PEG-PPG-PEG nanoparticles encapsulating the fluorescent dye, Rhodamine B in MCF-7 cells.

FIG. 6A shows the in-vitro release of encapsulated L-NuBCP-9 over time from the PLA-PEG-PPG-PEG nanoparticles synthesized using different copolymers at 25° C.

FIG. 6B shows the lack of efficacy of PLA-PEG-PPG-PEG nanoparticles synthesized using different block copolymers loaded with L-NuBCP-9 in normal HUVEC cells, as a negative control.

FIG. 7A shows the lack of efficacy of the anticancer peptide, L-NuBCP-9-loaded PLA-PEG-PPG-PEG nanoparticles on another primary HUVEC cell line.

FIG. 7B shows the efficacy of the delivery of the PLA-PEG-PPG-PEG nanoparticles loaded with anticancer peptide, L-NuBCP-9, compared with drug delivery using cell penetrating peptide (CPP) on MCF-7 cell proliferation.

FIG. 8A shows levels of hemoglobin in BALB/c mice treated with plain PLA-PEG-PPG-PEG nanoparticles to define any general toxicity by doing blood chemistry at a dose of 150 mg/kg body weight.

FIG. 8B shows levels of neutrophils and lymphocyte count in BALB/c mice treated with plain PLA-PEG-PPG-PEG nanoparticles to define any general toxicity by doing blood chemistry at a dose of 150 mg/kg body weight.

FIG. 8C shows packed cell volume, MCV (Mean Corpuscular Volume), MCH (Mean Corpuscular Hemoglobin) and MCHC (Mean Corpuscular Hemoglobin Concentration), in BALB/c mice treated with plain PLA-PEG-PPG-PEG nanoparticles to define any general toxicity by doing blood chemistry at a dose of 150 mg/kg body weight.

FIG. 9A shows the levels of aspartate transaminase and alanine transaminase in BALB/c mice treated with plain PLA-PEG-PPG-PEG nanoparticles to define any general toxicity by doing blood chemistry at a dose of 150 mg/kg body weight.

FIG. 9B shows the levels alkaline phosphatase in BALB/c mice treated with plain PLA-PEG-PPG-PEG nanoparticles to define any general toxicity by doing blood chemistry at a dose of 150 mg/kg body weight.

FIG. 9C shows the levels of urea and blood urea nitrogen (BUN) in BALB/c mice treated with plain PLA-PEG-PPG-PEG nanoparticles to define any general toxicity by doing blood chemistry at a dose of 150 mg/kg body weight.

FIG. 10 shows the histopathology of the brain, heart, liver, spleen, kidney and lung of BALB/c mice injected with plain PLA-PEG-PPG-PEG nanoparticles to define any general toxicity by doing histopathology of different organs.

FIG. 11A and FIG. 11B show tumor regression in Ehrlich Ascites Tumor (EAT) mice treated with LNuBCP-9-encapsulated PLA-PEG-PPG-PEG nanoparticles (8,800 g/mol).

FIG. 12A shows the Ehrlich Ascites Tumor in BALB-c mice at day 1.

FIG. 12B shows tumor growth suppression in EAT mice treated with L-NuBCP-9-encapsulated PLA-PEG-PPG-PEG nanoparticles (8,800 g/mol) at day 21.

FIG. 12C shows untreated, control mice at day 21.

FIG. 13 shows the efficacy of insulin-loaded PLA-PEG-PPG-PEG nanoparticles on controlling blood glucose levels in diabetic rabbits.

FIG. 14 shows the release data of a MUC1 cytoplasmic domain peptide linked to a polyarginine sequence (RRRRRRRRRCQCRRKN) from PLA-PEG-PPG-PEG nanoparticles.

FIG. 15A shows the SEM of PLA72K-PEG-PPG-PEG12K NPs.

FIG. 15B shows the TEM of PLA72K-PEG-PPG-PEG12K NPs.

FIG. 16 shows cellular internalization of Rhodamine B loaded PLA72K-PEG-PPG-PEG12K NPs.

FIG. 17A shows paclitaxel (also referred to herein as “PTX”) release from PLA-PEG-PPG-PEG NPs.

FIG. 17B shows L-NuBCP-9 release from PLA-PEG-PPG-PEG NPs.

FIG. 17C shows PTX and L-NuBCP-9 release from dual/hybrid PLA-PEG-PPG-PEG NPs encapsulating both the drugs in same nanoparticles.

FIG. 18A shows the treatment of MCF-7 cells (left panel) and MDA-MB-231 (right panel) cells upon exposure to NPs encapsulated with different ratios of PTX:NuBCP-9 (3:1, 1:1 and 1:3). After 72 h, the cells were analyzed by XTT assays The results are represented as percentage viability (mean±SD of three independent experiments).

FIG. 18B shows a time dependent study of dual loaded NPs (i.e., polymeric NPs comprising PTX and NuBCP-9) in comparison with single loaded NPs, where the time points are 0 hour (1); 12 hours post treatment (2); 24 hours post treatment (3); 48 hours post treatment (4) and 72 hours post treatment (5) using hormone-dependent breast carcinoma cell line MCF-7

FIG. 18C shows the proliferation inhibition of MCF-7 cells of a single formulation in comparison with free or single loaded NPs using different concentrations of the drugs.

FIG. 18D shows the proliferation inhibition of MDA-MB231 cells of a single formulation in comparison with free or single loaded NPs using different concentrations of the drugs.

FIG. 18E shows CI (combination index) for paclitaxel and L-NuBCP-9 analysis in connection with synergy in inhibition of MCF7 cells. The CI of less than 1.0 shows synergy. The CI numbers achieved in this analysis were 0.1 to 0.3 at different doses which demonstrate very high synergy in killing of MCF-7 cells.

FIG. 18F shows CI (combination index) for paclitaxel and L-NuBCP-9 analysis in connection with synergy in inhibition of MDA-MB-231 cells The CI numbers achieved in this analysis were 0.1 to 1.0 at different doses which demonstrate significantly high synergy in killing of MCF-7 cells.

FIG. 18G shows MCF-7 cells treated with different concentrations of empty NPs (circles), PTX/NPs (triangles) or NuBCP-9/NPs (squares) for 72 h. Cell viability was determined by XTT assays. The results are represented in the left panel as a percentage viability (mean+SD of three independent experiments). The indicated cells were treated with different concentrations of empty NPs (circles), PTX/NPs+NuBCP-9/NPs (squares) or PTX-NuBCP-9/NPs (triangles) for 72 h. Cell viability was determined by XTT assays. The results are represented in the right panel as a percentage viability (mean+SD of three independent experiments).

FIG. 18H shows MDA-MB-231 cells were treated with different concentrations of empty NPs (circles), PTX/NPs (triangles) or NuBCP-9/NPs (squares) for 72 h. Cell viability was determined by XTT assays. The results are represented in the left panel as a percentage viability (mean+SD of three independent experiments). The indicated cells were treated with different concentrations of empty NPs (circles), PTX/NPs+NuBCP-9/NPs (squares) or PTX-NuBCP-9/NPs (triangles) for 72 h. Cell viability was determined by XTT assays. The results are represented in the right panel as a percentage viability (mean+SD of three independent experiments).

FIG. 18I shows the combination index upon treatment of MCF-7 cells with the indicated concentrations of PTX/NPs alone, the indicated concentrations of NuBCP-9/NPs alone, and the indicated concentrations of PTX-NuBCP-9/NPs for 72 hours. Mean cell survival was assessed in triplicate by XTT assays. Numbers 1 to 7 in the graphs (left) represent combinations listed in tables (right). Fa indicates fraction affected and CI represents combination index.

FIG. 18J shows the combination index upon treatment of MDA-MB-231 cells with the indicated concentrations of PTX/NPs alone, the indicated concentrations of NuBCP-9/NPs alone, and the indicated concentrations of PTX-NuBCP-9/NPs for 72 hours. Mean cell survival was assessed in triplicate by XTT assays. Numbers 1 to 7 in the graphs (left) represent combinations listed in tables (right). Fa indicates fraction affected and CI represents combination index.

FIG. 19A shows the effects of PTX and NuBCP-9 (single/dual) loaded nanoparticles (NPs) on induction of cell death. A, confocal laser scanning microscopic images of Annexin V/PI double staining of MCF-7 cells left untreated (control; Top), treated with NuBCP-9 loaded PLA^{72K}-PEG-PPG-PEG NPs (second in middle), PTX loaded PLA^{72K}-PEG-PPG-PEG nanoparticles (third in middle), only free PTX as control (second in bottom) and, PTX-NuBCP-9 loaded PLA^{72K}-PEG-PPG-PEG Nps (bottom) for the indicated times.

FIG. 19B shows the percent of positive cells in early apoptosis, late apoptosis, or that have died upon exposure to L-NuBCP-9/PTX combination NPs, NuBCP-9 NPs, PTX NPs, PTX, and NPs.

FIG. 19C shows the Western blot data used to determine levels of BCL-2, Tubulin, cleaved form of caspase 3, and cleaved form of PARP proteins in MCF-7 cells.

FIG. 19D shows the levels of BCL-2, Tubulin, cleaved form of caspase 3, and cleaved form of PARP proteins in the breast cancer cell line as determined by the Western blott analysis shown in FIG. 19C.

FIG. 20A shows tumor growth curves (EAT syngeneic tumor model) generated from weekly and bi-weekly i.p L-NuBCP-9 peptide in combination with paclitaxel (PTX) loaded in NPs. Tumor growth curves showed that the bi-weekly i.p L-NuBCP-9 peptide in combination with paclitaxel (PTX) loaded in nanoparticles was effective in controlling EAT tumor growth as compared to untreated or weekly dosing. Each point represented the average of the volume of all tumor EAT mice±SE. *P<0.01, significantly different from the control PBS group; **P<0.001, significantly different from the control PBS groups; P<0.001, significantly different from peptide or paclitaxel alone group.

FIG. 20B shows tumor growth curves (EAT syngeneic tumor model) generated from bi-weekly i.p. paclitaxel (PTX) loaded in NPs. Tumor growth curves showed that the bi-weekly i.p L-NuBCP-9 paclitaxel (PTX) loaded in nanoparticles was effective in controlling EAT tumor growth as compared to untreated or weekly dosing.

FIG. 20C shows tumor growth curves generated from bi-weekly i.p. NuBCP-9 peptide loaded in NPs. Tumor growth curves showed that the bi-weekly i.p. L-NuBCP-9 peptide loaded in nanoparticles was effective in controlling EAT tumor growth as compared to untreated or weekly dosing.

FIG. 21 shows histopathology of tumor tissues obtained from mice treated with the control, PTX control, PTX loaded NPs, L-NuBCP-9 loaded NPs and Dual Drug loaded NPs (right to left) for 21 days and stained with hematoxylin and eosin (X400). Very low Ki67 expression is seen in the combination test; reduced ki67 expression in L-NuBCP-9 loaded Nps and PTX loaded Nps while high expression is seen in vehicle control and PTX control (P<0.05). TUNEL-positive cells are seen maximally in the combination drug loaded NPs, some TUNEL-positive cells are seen L-NuBCP-9 loaded Nps and PTX loaded Nps while no TUNEL-positive cells are seen in the vehicle control (P<0.05).

FIG. 22 shows antitumor activity of PTX and L-NuBCP-9 (single/dual) loaded nanoparticles. Ehrlich tumor-bearing mice were treated with empty NPs (i.p., squares, twice weekly), 10 mg/kg L-NuBCP-9 loaded NPs (i.p., triangles, twice weekly), 10 mg/kg PTX loaded NPs (i.p., diamonds, twice weekly), or 10 mg/kg PTX-NuBCP-9 dual drug loaded NPs (i.p., circles, twice weekly) for a 21-day cycle. Tumor measurements were performed on the indicated days. The results are expressed as tumor volumes (mean+SD).

FIG. 23 shows the results in the experiment described in FIG. 22 expressed as the percentage survival as determined by Kaplan-Meier analysis empty NPs (squares), L-NuBCP-9 loaded NPs (triangles), PTX loaded NPs (circles), and PTX-NuBCP-9 loaded NPs (open squares). The statistical analysis was performed between the vehicle control and the PTX-NuBCP-9 loaded nanoparticle group (P<0.001).

FIG. 24 shows antitumor activity of PTX and L-NuBCP-9 (single/dual) loaded nanoparticles at the dose of 30 mg/kg. Syngeneic EAT model comparing Paclitaxel/NP, L-NuBCP-9/NP with Paclitaxel+NuBCP-9 Dual/NP 30 mg/kg IP weekly dosing×3.

FIG. 25 shows a colocalization study of MCF-7 cells treated with FITC-labeled L-NuBCP-9 nanoparticles for 12 h. After washing, the cells were fixed and visualized by confocal microscopy. Mitochondria were stained with mitochondria selective Mitotracker dye. (upper panel). Separately, MCF-7 cells were treated with NPs encapsulating L-NuBCP-9-Rho B and paclitaxel labeled with green fluoro dye (FITC) for 12 h. After washing, the cells were fixed and visualized by confocal microscopy. Colocalization of L-NuBCP-9 and PTX were seen in mitochondria (lower panel).

FIG. 26 shows a schematic presentation of PTX-NuBCP-9 dual loaded NPs, acting on multiple targets, to show synergistic effect.

FIG. 27A shows analysis of whole cell lysates from wild-type MCF-7 (MCF-7) and PTX-resistant MCF-7 (MCF-7/PTX-R) by immunoblotting with anti-P gp1, anti-BCL-2 and anti-β-actin antibodies (see Example 9).

FIG. 27B shows MCF-7 or MCF-7/PTX-R cells that were treated with 100 nM PTX or 100 nM PTX/NPs for 12 h. After washing, the cells were fixed and visualized by confocal microscopy (see Example 9).

FIG. 27C shows confocal laser scanning microscopic images of MCF-7 (top 2 panels) and MCF-7/PTX-R (bottom 2 panels) cells treated with 100 nM PTX or 100 nM PTX/NPs for 48 h and then stained with AnnexinV/PI (see Example 9).

FIG. 27D shows MCF-7 and MCF-7/PTX-R cells that were treated with 100 nM PTX or 100 nM PTX/NPs for 48 h. Cells were then stained with Annexin V/PI and analyzed by FACS. The percentage of PI+ and/or annexin V+ cells is included in the panels. (see Example 9).

FIG. 27E shows whole cell lysates from MCF-7 and MCF-7/PTX-R that were treated with 100 nM PTX, 100 nM nab-paclitaxel (nab-PTX; Abraxane) or 100 nM PTX/NPs for 48 h. Analysis was performed by immunoblotting with anti-caspase-3 CF, anti-PARP CF and anti-β-Actin antibodies (see Example 9).

FIG. 27F shows analysis of whole cell lysates from MCF-7/PTX-R cells treated with 100 nM PTX-NuBCP-9/NPs for 72 h. Analysis was performed by immunoblotting with anti-P-gp, anti-BCL-2, and anti-β-Actin antibodies (see Example 9).

### DETAILED DESCRIPTION

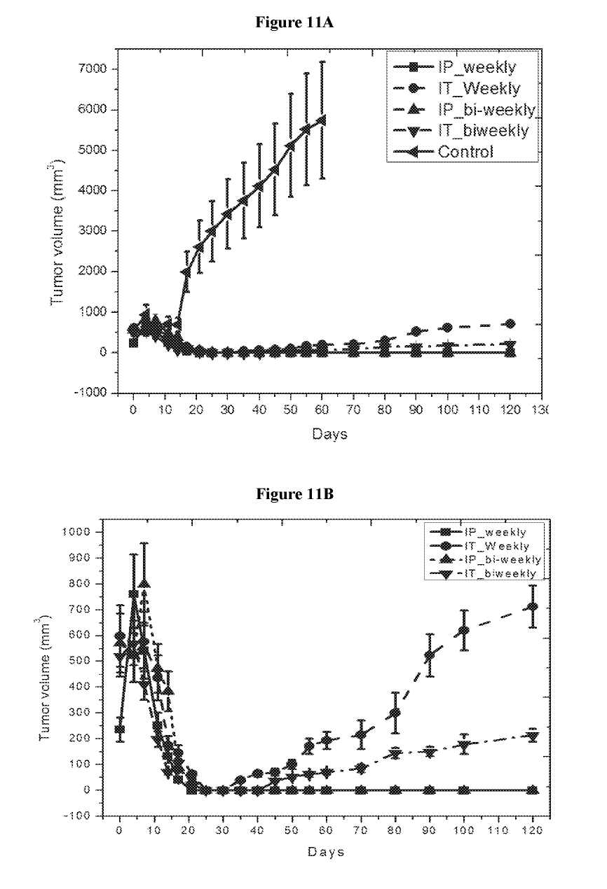

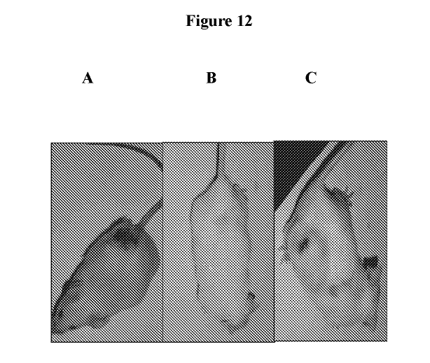

NuBCP-9 is a highly promising anti-cancer peptide which selectively induces apoptosis of cancer cells by exposing the BCL-2 BH3 domain and blocking the BCL-xL survival function (Kolluri S K, et al. A short Nur77-derived peptide converts Bcl-2 from a protector to a killer. Cancer Cell 2008; 14:285-98). NuBCP-9 was linked to the D-Arg octamer r8 for intracellular delivery, a modification that has been reported to decrease selectivity by inducing BCL-2-independent cell killing involving membrane disruption. The sustained delivery of L-NuBCP-9 peptide via a novel polymeric PLA-PEG-PPG-PEG nanoparticle administered i.p. was effective in inducing complete regressions of the Ehrlich tumors (see, e.g., FIG. 11 and FIG. 12, as well as Example 7). Characteristics and processes for preparing this type of nanoparticle is disclosed in WO 2013/160773, the content of which is hereby incorporated by reference in its entirety.

Nanoparticles (also referred to herein as “NPs”) can be produced as nanocapsules or nanospheres. Protein loading in the nanoparticle can be carried out by either the adsorption process or the encapsulation process (Spada et al., 2011; Protein delivery of polymeric nanoparticles; World Academy of Science, Engineering and Technology: 76). Nanoparticles, by using both passive and active targeting strategies, can enhance the intracellular concentration of drugs in cancer cells while avoiding toxicity in normal cells. When nanoparticles bind to specific receptors and enter the cell, they are usually enveloped by endosomes via receptor-mediated endocytosis, thereby bypassing the recognition of P-glycoprotein, one of the main drug resistance mechanisms (Cho et al., 2008, Therapeutic Nanoparticles for Drug Delivery in Cancer, Clin. Cancer Res., 2008, 14:1310-1316). Nanoparticles are removed from the body by opsonization and phagocytosis (Sosnik et al., 2008; Polymeric Nanocarriers: New Endeavors for the Optimization of the Technological Aspects of Drugs; Recent Patents on Biomedical Engineering, 1: 43-59). Nanocarrier based systems can be used for effective drug delivery with the advantages of improved intracellular penetration, localized delivery, protect drugs against premature degradation, controlled pharmacokinetic and drug tissue distribution profile, lower dose requirement and cost effectiveness (Farokhzad O C, et al.; Targeted nanoparticle-aptamer bioconjugates for cancer chemotherapy in vivo. Proc. Natl. Acad. Sci. USA 2006, 103 (16): 6315-20; Fonseca C, et al., Paclitaxel-loaded PLGA nanoparticles: preparation, physicochemical characterization and in vitro anti-tumoral activity. J. Controlled Release 2002; 83 (2): 273-86; Hood et al., Nanomedicine, 2011, 6(7):1257-1272).

The uptake of nanoparticles is indirectly proportional to their small dimensions. Due to their small size, the polymeric nanoparticles have been found to evade recognition and uptake by the reticulo-endothelial system (RES), and can thus circulate in the blood for an extended period (Borchard et al., 1996, Pharm. Res. 7: 1055-1058). Nanoparticles are also able to extravasate at the pathological site like the leaky vasculature of a solid tumor, providing a passive targeting mechanism. Due to the higher surface area leading to faster solubilization rates, nano-sized structures usually show higher plasma concentrations and area under the curve (AUC) values. Lower particle size helps in evading the host defense mechanism and increase the blood circulation time. Nanoparticle size affects drug release. Larger particles have slower diffusion of drugs into the system. Smaller particles offer larger surface area but lead to fast drug release. Smaller particles tend to aggregate during storage and transportation of nanoparticle dispersions. Hence, a compromise between a small size and maximum stability of nanoparticles is desired. The size of nanoparticles used in a drug delivery system should be large enough to prevent their rapid leakage into blood capillaries but small enough to escape capture by fixed macrophages that are lodged in the reticuloendothelial system, such as the liver and spleen.

In addition to their size, the surface characteristics of nanoparticles are also an important factor in determining the life span and fate during circulation. Nanoparticles should ideally have a hydrophilic surface to escape macrophage capture. Nanoparticles formed from block copolymers with hydrophilic and hydrophobic domains meet these criteria. Controlled polymer degradation also allows for increased levels of agent delivery to a diseased state. Polymer degradation can also be affected by the particle size. Degradation rates increase with increase in particle size in vitro (Biopolymeric nanoparticles; Sundar et al., 2010, Science and Technology of Advanced Materials; doi:10.1088/1468-6996/11/1/014104).

Poly(lactic acid) (PLA) has been approved by the US FDA for applications in tissue engineering, medical materials and drug carriers and poly(lactic acid)-poly(ethylene glycol) PLA-PEG based drug delivery systems are known in the art. US2006/0165987A1 describes a stealthy polymeric biodegradable nanosphere comprising poly(ester)-poly(ethylene) multiblock copolymers and optional components for imparting rigidity to the nanospheres and incorporating pharmaceutical compounds. US2008/0081075A1 discloses a novel mixed micelle structure with a functional inner core and hydrophilic outer shells, self-assembled from a graft macromolecule and one or more block copolymer. US2010/0004398A1 describes a polymeric nanoparticle of shell/core configuration with an interphase region and a process for producing the same.

However, these polymeric nanoparticles essentially require the use of about 1% to 2% emulsifier for the stability of the nanoparticles. Emulsifiers stabilize the dispersed particles in a medium. PVA, PEG, Tween 80 and Tween 20 are some of the common emulsifiers. The use of emulsifiers is however, a cause of concern for in vivo applications as the leaching out of emulsifiers can be toxic to the subject (Safety Assessment on polyethylene glycols (PEGS) and their derivatives as used in cosmetic products, Toxicology, 2005 Oct. 15; 214 (1-2): 1-38). The use of emulsifier also increases the mass of the nanoparticle thereby reducing the drug load, leading to higher dosage requirements. Other disadvantages still prevalent in the nanoparticle drug carrier systems are poor oral bioavailability, instability in circulation, inadequate tissue distribution and toxicity. A delivery system that can effectively deliver therapeutic agents including therapeutic peptides such as NuBCP-9 into the cytosol of diseased (e.g., cancerous) cells without the disadvantages presented above is described herein.

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The articles “a,” “an,” and “the” are used to refer to one or to more than one (i.e., to at least one) of the grammatical object of the article.

The terms “comprise” “comprising” “including” “containing” “characterized by” and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

As used herein, the term “about” or “approximately” usually means within 20%, more preferably within 10%, and most preferably still within 5% of a given value or range.

The term “biodegradable” as used herein refers to both enzymatic and non-enzymatic breakdown or degradation of the polymeric structure.

As used herein, the term “nanoparticle” refers to particles in the range between 10 nm to 1000 nm in diameter, wherein diameter refers to the diameter of a perfect sphere having the same volume as the particle. The term “nanoparticle” is used interchangeably as “nanoparticle(s)”. In some cases, the diameter of the particle is in the range of about 1-1000 nm, 10-500 nm, 30-270 nm, 30-200 nm, or 30-120 nm.

In some cases, a population of particles may be present. As used herein, the diameter of the nanoparticles is an average of a distribution in a particular population.

As used herein, the term “polymer” is given its ordinary meaning as used in the art, i.e., a molecular structure comprising one or more repeat units (monomers), connected by covalent bonds. The repeat units may all be identical, or in some cases, there may be more than one type of repeat unit present within the polymer.

The term “nucleic acid” refers to polynucleotides such as deoxyribonucleic acid (DNA), ribonucleic acid (RNA), its variants and derivatives thereof.

As used herein, the term “therapeutic agent” and “drug” are used interchangeably and are also intended to encompass not only compounds or species that are inherently pharmaceutically or biologically active, but materials which include one or more of these active compounds or species, as well as conjugations, modification, and pharmacologically active fragments, and antibody derivatives thereof.

A “targeting moiety” or “targeting agent” is a molecule that will bind selectively to the surface of targeted cells. For example, the targeting moiety may be a ligand that binds to the cell surface receptor found on a particular type of cell or expressed at a higher frequency on target cells than on other cells.

The targeting agent, or therapeutic agent can be a peptide or protein. “Proteins” and “peptides” are well-known terms in the art, and as used herein, these terms are given their ordinary meaning in the art. Generally, peptides are amino acid sequences of less than about 100 amino acids in length, but can include up to 300 amino acids. Proteins are generally considered to be molecules of at least 100 amino acids. The amino acids can be in D- or L-configuration. A protein can be, for example, a protein drug, an antibody, a recombinant antibody, a recombinant protein, an enzyme, or the like. In some cases, one or more of the amino acids of the peptide or protein can be modified, for example by the addition of a chemical entity such as a carbohydrate group, a phosphate group, a farnesyl group, an isofarnesyl group, a fatty acid group, a linker for conjugation, functionalization, or other modification such as cyclization, by-cyclization and any of numerous other modifications intended to confer more advantageous properties on peptides and proteins. In other instances one or more of the amino acids of the peptide or protein can be modified by substitution with one or more non-naturally occurring amino acids. The peptides or proteins may by selected from a combinatorial library such as a phage library, a yeast library, or an in vitro combinatorial library.

As used herein, the term “antibody” refers to any molecule incorporating an amino acid sequence or molecule with secondary or tertiary structural similarity conferring binding affinity to a given antigen that is similar or greater to the binding affinity displayed by an immunoglobulin variable region containing molecule from any species. The term antibody includes, without limitation native antibodies consisting of two heavy chains and two light chains; binding molecules derived from fragments of a light chain, a heavy chain, or both, variable domain fragments, heavy chain or light chain only antibodies, or any engineered combination of these domains, whether monospecific or bispecific, and whether or not conjugated to a second diagnostic or therapeutic moiety such as an imaging agent or a chemotherapeutic molecule. The term includes without limitation immunoglobulin variable region derived binding moieties whether derived from a murine, rat, rabbit, goat, llama, camel, human or any other vertebrate species. The term refers to any such immunoglobulin variable region binding moiety regardless of discovery method (hybridoma-derived, humanized, phage derived, yeast derived, combinatorial display derived, or any similar derivation method known in the art), or production method (bacterial, yeast, mammalian cell culture, or transgenic animal, or any similar method of production known in the art).

The term “combination,” “therapeutic combination,” or “pharmaceutical combination” as used herein refer to the combined administration of two or more therapeutic agents. (e.g., co-delivery).

The term “pharmaceutically acceptable” as used herein refers to those compounds, materials, compositions and/or dosage forms, which are, within the scope of sound medical judgment, suitable for contact with the tissues a warm-blooded animal, e.g., a mammal or human, without excessive toxicity, irritation allergic response and other problem complications commensurate with a reasonable benefit/risk ratio.

A “therapeutically effective amount” of a polymeric nanoparticle comprising one or more therapeutic agents is an amount sufficient to provide an observable or clinically significant improvement over the baseline clinically observable signs and symptoms of the disorders treated with the combination.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest and/or reduce the risk of worsening a disease. The term “prevent”, “preventing” or “prevention” as used herein comprises the prevention of at least one symptom associated with or caused by the state, disease or disorder being prevented.

### Polymeric Nanoparticles

Provided herein is a non-toxic, safe, biodegradable polymeric nanoparticle made up of block copolymer for the delivery of one or more therapeutics. The biodegradable polymeric nanoparticles of the instant invention are formed of a block copolymer consisting essentially of poly(lactic acid) (PLA) chemically modified with a hydrophilic-hydrophobic block copolymer, wherein said hydrophilic-hydrophobic block copolymer is selected from poly(methyl methacrylate)-poly(methylacrylic acid) (PMMA-PMAA), poly(styrene)-poly(acrylic acid) (PS-PAA), poly(acrylic acid)-poly(vinylpyridine) (PAA-PVP), poly(acrylic acid)-poly(N,N-dimethylaminoethyl methacrylate) (PAA-PDMAEMA), poly(ethylene glycol)-poly(butylene glycol) (PEG-PBG), and poly(ethylene glycol)-poly(propylene glycol)-poly(ethylene glycol) (PEG-PPG-PEG).

As used herein, the “polymeric nanoparticle of the invention” refers to polymeric nanoparticles formed of a block copolymer comprising poly(lactic acid) (PLA) chemically modified with a hydrophilic-hydrophobic block copolymer, wherein said hydrophilic-hydrophobic block copolymer is selected from poly(methyl methacrylate)-poly(methylacrylic acid) (PMMA-PMAA), poly(styrene)-poly(acrylic acid) (PS-PAA), poly(acrylic acid)-poly(vinylpyridine) (PAA-PVP), poly(acrylic acid)-poly(N,N-dimethylaminoethyl methacrylate) (PAA-PDMAEMA), poly(ethylene glycol)-poly(butylene glycol) (PEG-PBG), and poly(ethylene glycol)-poly(propylene glycol)-poly(ethylene glycol) (PEG-PPG-PEG). Thus, the “polymeric nanoparticle of the invention” encompasses polymeric nanoparticles formed of a block copolymer comprising or consisting essentially of poly(lactic acid) (PLA) chemically modified with poly(ethylene glycol)-poly(propylene glycol)-poly(ethylene glycol) (PEG-PPG-PEG).

The present invention provides a process for preparing the biodegradable polymeric nanoparticle comprising one or more therapeutics. The resulting nanoparticle is not only non-toxic, safe, and biodegradable, but is stable in vivo, has high storage stability and can be safely used in a nanocarrier system or drug delivery system in the field of medicine. In fact, the nanoparticles of the instant invention increase the half-life of the deliverable drug or therapeutic agent in-vivo.

The present invention also provides a process for efficient drug loading (e.g., a peptide comprising NuBCP-9 as a single agent, or NuBCP-9 and a chemotherapeutic agent or a targeted anti-cancer agent) on a biodegradable polymeric nanoparticle to form an effective and targeted drug delivery nanocarrier system which prevents premature degradation of active agents and has a strong potential for use in cancer therapy.

There is also provided a composition comprising the biodegradable polymeric nanoparticle for use in medicine and in other fields that employ a carrier system or a reservoir or depot of nanoparticles. The nanoparticles of the present invention can be extensively used in prognostic, therapeutic, diagnostic or theranostic compositions. Suitably, the nanoparticles of the present invention are used for drug and agent delivery, as well as for disease diagnosis and medical imaging in human and animals. Thus, the instant invention provides a method for the treatment of disease using the nanoparticles further comprising a therapeutic agent as described herein. The nanoparticles of the present invention can also be use in other applications such as chemical or biological reactions where a reservoir or depot is required, as biosensors, as agents for immobilized enzymes and the like.

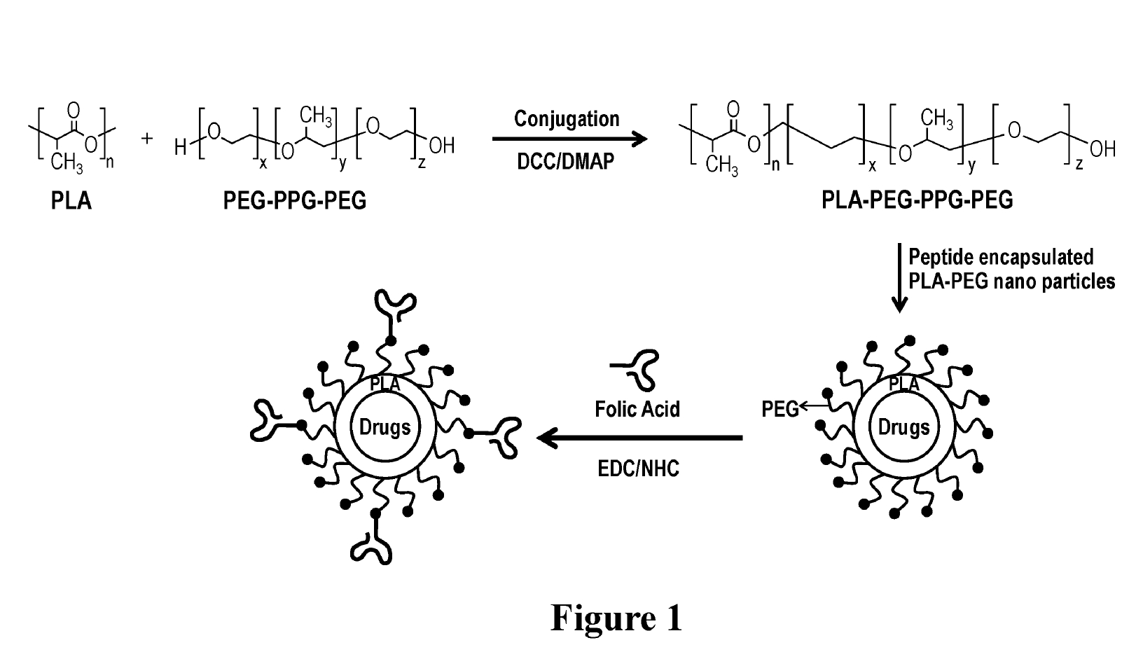

Unexpected and surprising results were obtained during production of biodegradable polymeric nanoparticles without the use of any emulsifiers or stabilizers according to the processes described herein. The biodegradable polymeric nanoparticles so obtained by the process are safe, stable and non-toxic. In an embodiment, the block copolymer PEG-PPG-PEG is covalently attached to the poly-lactic acid (PLA) matrix, resulting in the block copolymer becoming a part of the matrix, i.e., the nanoparticle delivery system. In contrast, in the prior art, the emulsifier (e.g. PEG-PPG-PEG) is not a part of the nanoparticle matrix and therefore leaches out (FIG. 1). In contrast to nanoparticles of the prior art, there is no leaching out of emulsifier into the medium from the nanoparticles provided herein.

The nanoparticles obtained by the present process are non-toxic and safe due to the absence of added emulsifiers, which can leach out in vivo. The absence or reduced quantity of emulsifier also leads to nanoparticles with a higher drug to polymer ratio. These nanoparticles have higher stability, and an increased storage shelf life as compared to the polymeric nanoparticles present in the art. The polymeric nanoparticles of the present invention are prepared to be biodegradable so that the degradation products may be readily excreted from the body. The degradation also provides a method by which the encapsulated contents in the nanoparticle can be released at a site within the body.

Poly(lactic acid) (PLA), is a hydrophobic polymer, and is the preferred polymer for synthesis of the polymeric nanoparticles of the instant invention. However, poly(glycolic acid) (PGA) and block coploymer of poly lactic acid-co-glycolic acid (PLGA) may also be used. The hydrophobic polymer can also be biologically derived or a biopolymer.

The molecular weight of the PLA used is generally in the range of about 2,000 g/mol to 80,000 g/mol. Thus, in an embodiment, the PLA used is in the range of about 2,000 g/mol to 80,000 g/mol. The average molecular weight of PLA may also be about 72,000 g/mol. As used herein, one g/mole is equivalent to one “dalton” (i.e., dalton and g/mol are interchangeable when referring to the molecular weight of a polymer.

Block copolymers like poly(ethylene glycol)-poly(propylene glycol)-poly(ethylene glycol) (PEG-PPG-PEG), poly(methyl methacrylate)-poly(methylacrylic acid) (PMMA-PMAA), poly(styrene)-poly(acrylic acid) (PS-PAA), poly(acrylic acid)-poly(vinylpyridine) (PAA-PVP), poly(acrylic acid)-poly(N,N-dimethylaminoethyl methacrylate) (PAA-PDMAEMA), poly(ethylene glycol)-poly(butylene glycol) (PEG-PBG) and PG-PR (Polyglycerol (PG) and its copolymers with polyester (PR) including adipic acid, pimelic acid and sebecic acid) are hydrophilic or hydrophilic-hydrophobic copolymers that can be used in the present invention and include ABA type block copolymers such as PEG-PPG-PEG, BAB block copolymers such as PPG-PEG-PPG, (AB)ₙ type alternating multiblock copolymers and random multiblock copolymers. Block copolymers may have two, three or more numbers of distinct blocks. PEG is a preferred component as it imparts hydrophilicity, anti-phagocytosis against macrophage and resistance to immunological recognition.

In some embodiments, the average molecular weight (Mn) of the hydrophilic-hydrophobic block copolymer is generally in the range of 1,000 to 20,000 g/mol. In a further embodiment, the average molecular weight (Mn) of the hydrophilic-hydrophobic block copolymer is about 4,000 g/mol to 15,000 g/mol. In some cases, the average molecular weight (Mn) of the hydrophilic-hydrophobic block copolymer is 4,400 g/mol, 8,400 g/mol, or 14,600 g/mol.

A block copolymer of the instant invention can consist essentially of a segment of poly(lactic acid) (PLA) and a segment of poly(ethylene glycol)-poly(propylene glycol)-poly(ethylene glycol) (PEG-PPG-PEG).

A specific biodegradable polymeric nanoparticle of the instant invention is formed of the block copolymer poly(lactic acid)-poly(ethylene glycol)-poly(propylene glycol)-poly(ethylene glycol) (PLA-PEG-PPG-PEG).

Another specific biodegradable polymeric nanoparticle of the instant invention is formed of the block copolymer poly(lactic acid)-poly(ethylene glycol)-poly(propylene glycol)-poly(ethylene glycol)-poly(lactic acid) (PLA-PEG-PPG-PEG-PLA).

The biodegradable polymers of the instant invention are formable by chemically modifying PLA with a hydrophilic-hydrophobic block copolymer using a covalent bond.

The biodegradable polymeric nanoparticles of the instant invention can have size in the range of about 30-300 nm. In a further embodiment, the biodegradable polymeric nanoparticles of the instant invention have a size in the range of about 30-120 nm.

In an embodiment, the biodegradable polymer of the instant invention is substantially free of emulsifier, or may comprise external emulsifier by an amount of about 0.5% to 5% by weight.

In an embodiment, the biodegradable polymeric nanoparticle of the present invention is PLA-PEG-PPG-PEG, and the average molecular weight of the poly(lactic acid) block is about 60,000 g/mol, the average weight of the PEG-PPG-PEG block is about 8,400 or about 14,600 g/mol, and the external emulsifier is about 0.5% to 5% by weight.

In another embodiment, the biodegradable polymeric nanoparticle of the present invention is PLA-PEG-PPG-PEG, and the an average molecular weight of the poly(lactic acid) block is less than or equal to approximately 16,000 g/mol, the average weight of the PEG-PPG-PEG block is about 8,400 g/mol or about 14,600 g/mol, and wherein the composition is substantially free of emulsifier.

### Preparation of Polymeric Nanoparticles

The process for preparing biodegradable polymeric nanoparticles of the instant invention comprises dissolving poly(lactic acid) (PLA) and a hydrophilic-hydrophobic block copolymer in an organic solvent to obtain a solution; adding a carbodiimide coupling agent and a base to the solution to obtain a reaction mixture; stirring the reaction mixture to obtain a block copolymer of PLA chemically modified with the hydrophilic-hydrophobic block copolymer; dissolving the block copolymer from the previous step in organic solvent and homogenizing to obtain a homogenized mixture; adding the homogenized mixture to an aqueous phase to obtain an emulsion; and stirring the emulsion to obtain the polymeric nanoparticles.

Carbodiimide coupling agents are well-known in the art. Suitable carbodiimide coupling agents include, but are not limited to, N,N-dicyclohexylcarbodiimide (DCC), N-(3-diethylaminopropyl)-N-ethylcarbodiimide (EDC), and N,N-diisopropylcarbodiimide.

The coupling reaction is usually carried out in the presence of catalysts and/or auxiliary bases such as trialkylamines, pyridine, or 4-dimethylamino pyridine (DMAP).

The coupling reaction can be also carried out in combination with a hydroxyderivative, such as N-hydroxysuccinimide (NHS). Other hydroxyderivatives include, but are not limited to, 1-hydroxybenzotriazole (HOBt), 1-hydroxy-7-azabenzotriazole (HOAt), 6-chloro-1-hydroxybenzotriazole (Cl-HOBt).

Organic solvents useful in the preparation of the nanoparticles prepared herein are suitably acetonitrile (C₂H₃N), dimethyl formamide (DMF; C₃H₇NO), acetone ((CH₃)₂CO) and dichloromethane (CH₂Cl₂).

The process described above can optionally comprise the additional steps of washing the biodegradable polymeric nanoparticles with water, and drying the polymeric biodegradable polymeric nanoparticles. The process can also optionally comprise a first step of adding emulsifier. The nanoparticles resulting from this process can have a size in the range of about 30-300 nm, or about 30-120 nm.

In a specific process, the PLA and the copolymer, PEG-PPG-PEG, are dissolved in an organic solvent to obtain a polymeric solution. To this solution, N,N-dicyclohexylcarbodiimide (DCC) is added followed by 4-dimethylaminopyridine (DMAP) at −4° C. to 0° C. The solution is allowed to stir at 250 to 300 rpm at a low temperature ranging from −4° C. to 0° C. for 20 to 28 hours. The nanoparticles of PLA-PEG-PPG-PEG have PLA covalently linked to PEG-PPG-PEG to form a PLA-PEG-PPG-PEG matrix. The nanoparticles are precipitated by an organic solvent like diethyl ether, methanol or ethanol and separated from the solution by conventional methods in the art including filtration, ultracentrifugation or ultrafiltration. The nanoparticles are stored in a temperature ranging from 2° C. to 8° C.

The process of the present invention provides the added advantage of not requiring additional steps of freezing or the use of decoy proteins as none, or a minimal amount, of emulsifiers are used in the process. The present invention is easily carried out in ambient room temperature conditions of 25° C.−30° C. and does not require excessive shearing to obtain the desired small particle size.

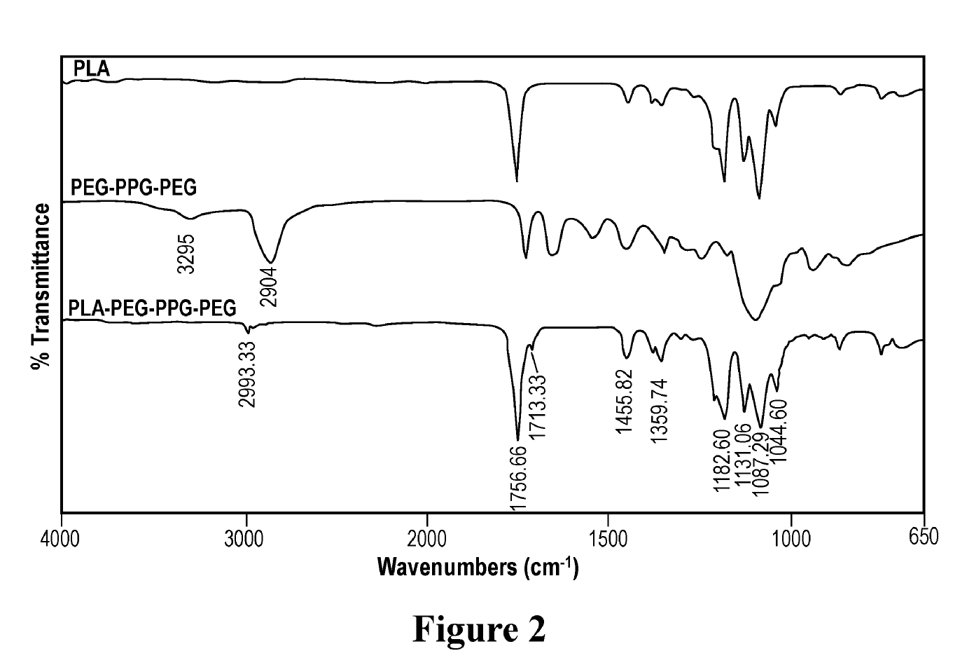

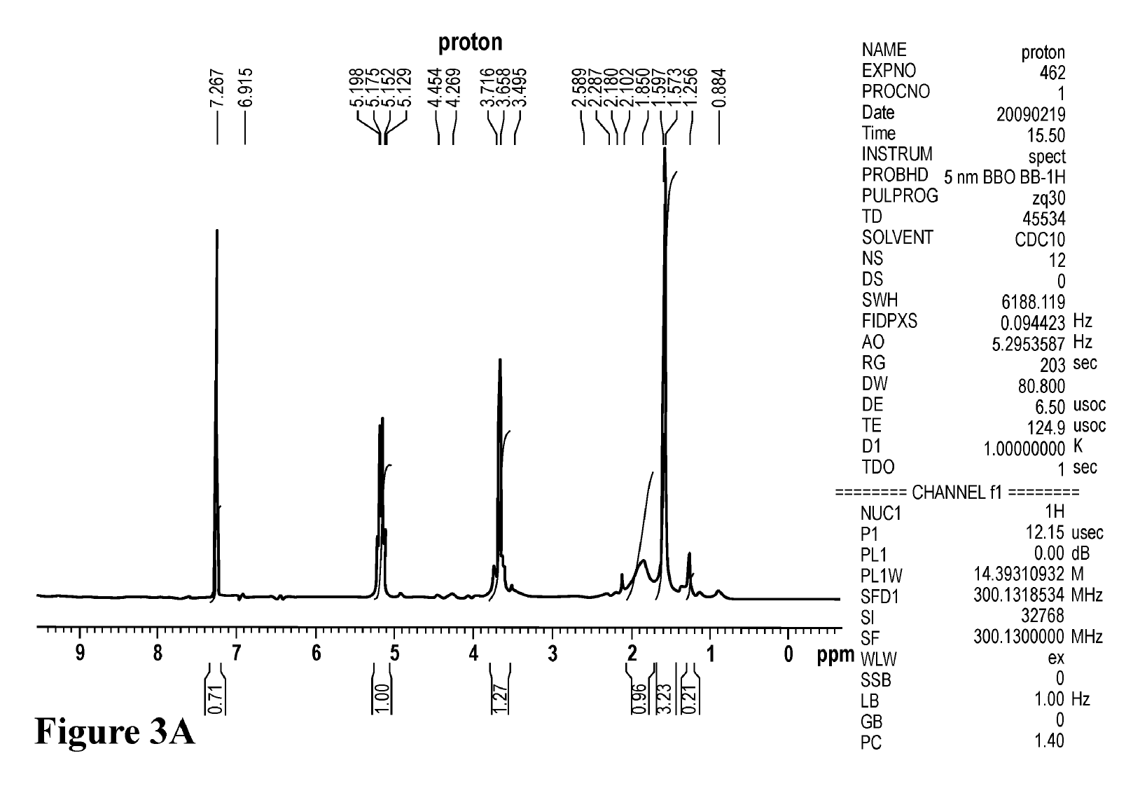

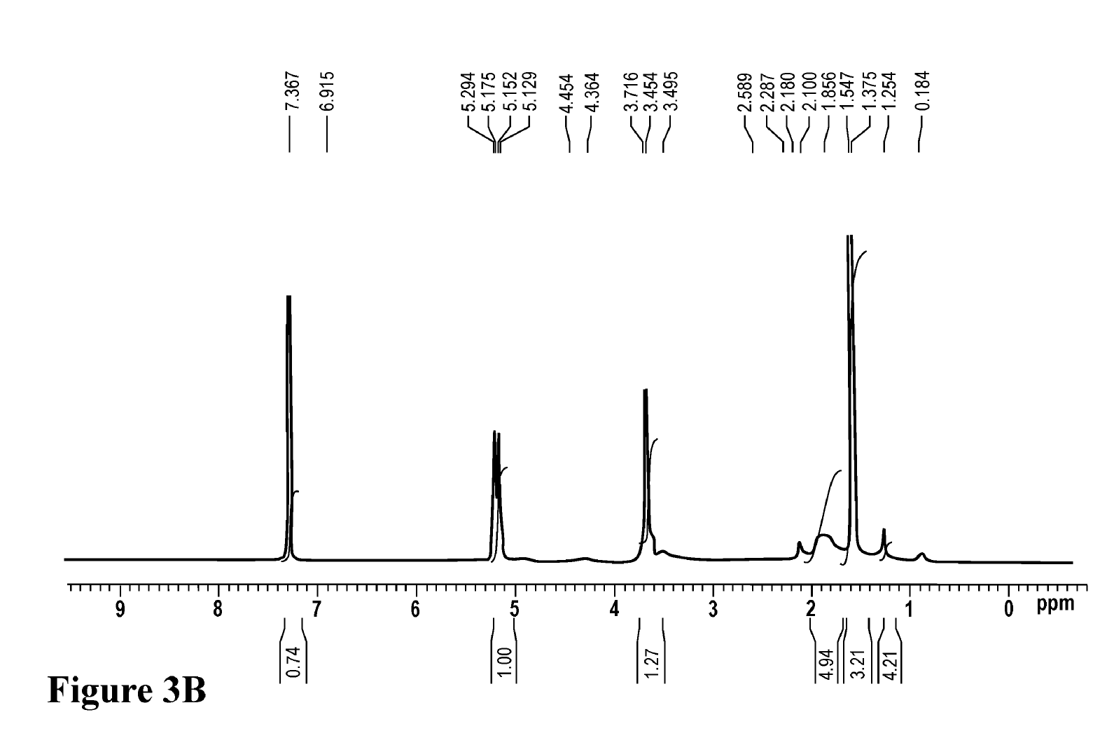

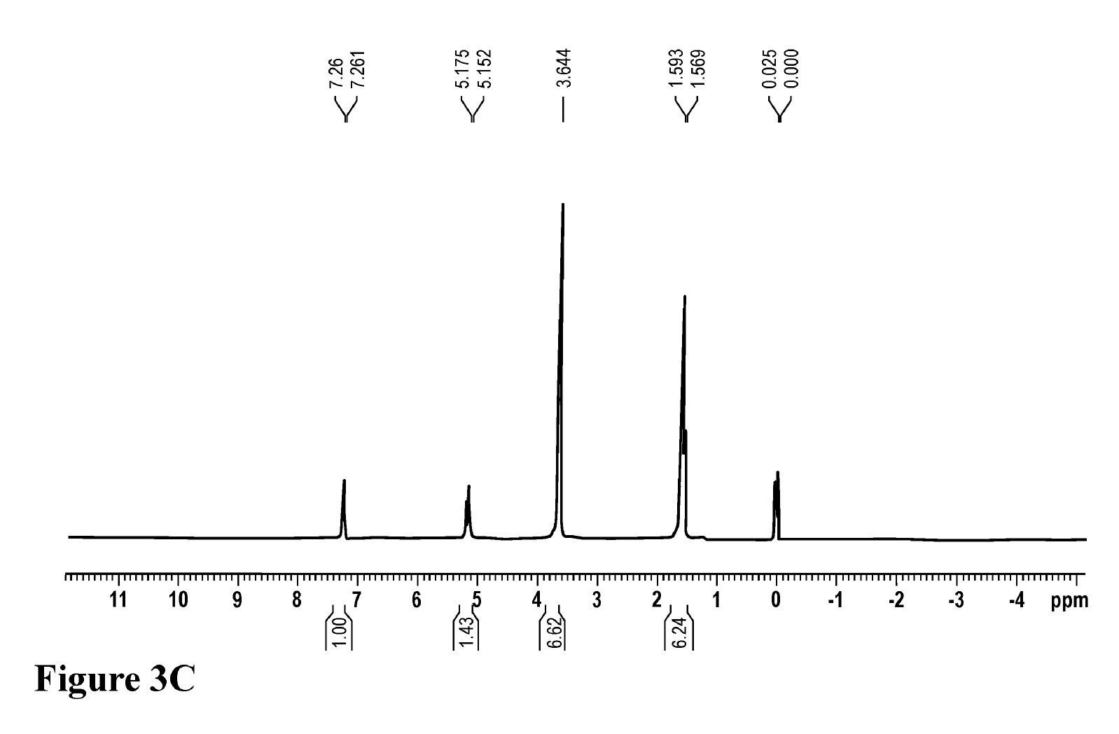

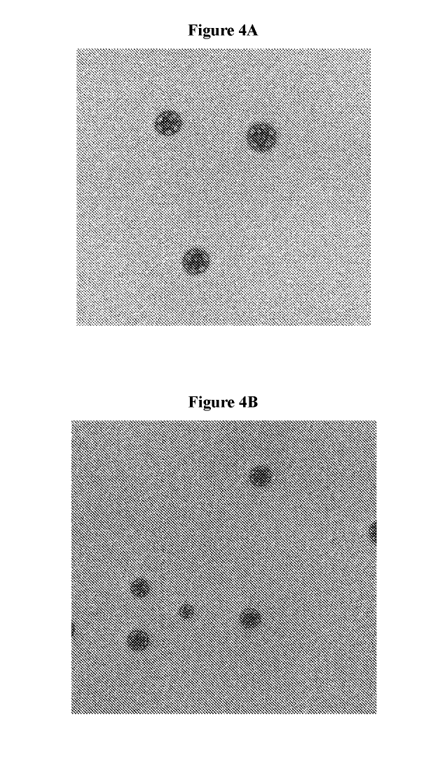

A FTIR spectrum of one example of nanoparticles of the present invention is provided in FIG. 2. The NMR spectra of the nanoparticles are provided in FIGS. 3A, 3B, and 3C. The nanoparticle is substantially spherical in configuration as shown in the TEM images of FIGS. 4A and 4B, however, the nanoparticles can adopt a non-spherical configuration upon swelling or shrinking. The nanoparticle is amphiphillic in nature. The zeta potential and PDI (Polydispersity Index) of the nanoparticles are provided in Table 2. Storage stability of the nanoparticles of the present invention is better compared to the conventional emulsifier based systems as there is no addition of any free emulsifiers to the process and the block copolymer comprising the PEG moiety is covalently linked in the overall PLA-PEG-PPG-PEG matrix. The storage shelf life of the nanoparticle ranges from 6 to 18 months.

The nanoparticles of the present invention can have dimensions ranging from 30-120 nm as measured using a Transmission Electron Microscope (FIG. 4). In suitable embodiments, the diameter of the nanoparticles of the present invention will be less than 500 nma in diameter, less than 300 nm in diameter, or less than 200 nm in diameter. In certain such embodiments, the nanoparticles of the present invention will be in the range of about 10 to 500 nm, about 10 to 300 nm, about 10 to 200 nm, in the range of about 20 to 150 nm, or in the range of about 30 to 120 nm in diameter.

Specific processes for nanoparticle formation and uses in pharmaceutical composition are provided herein for purpose of reference. These processes and uses may be carried out through a variety of methods apparent to those of skill in the art.

In an embodiment of the present invention, provided herein is a process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer.

In another embodiment of the present invention, there is provided a process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer, wherein said process optionally comprises the steps of washing the nanoparticles of PLA-PEG-PPG-PEG block copolymer with water and drying the nanoparticles by conventional method.

In another embodiment of the present invention, there is provided a process for preparation of biodegradable polymeric nanoparticles of PLA-PEG-PPG-PEG block copolymer, wherein said process comprises (a) dissolving a PEG-PPG-PEG copolymer and poly-lactic acid (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer, wherein size of the nanoparticle is in the range of about 30 to 300 nm or about 30-120 nm.

In yet another embodiment of the present invention, there is provided a process for preparation of biodegradable polymeric nanoparticles of PLA-PEG-PPG-PEG block copolymer, wherein said process comprises (a) dissolving a PEG-PPG-PEG copolymer and poly-lactic acid (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer, wherein molecular weight of the PEG-PPG-PEG copolymer is in the range of 1,000 g/mol to 10,000 g/mol.

In a further embodiment of the present invention, there is provided a process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer, wherein molecular weight of PLA is in the range of 10,000 g/mol to 60,000 g/mol.

In a further embodiment of the present invention, there is provided a process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer, wherein the solution of step (a) optionally comprises additives such as emulsifier.

Another embodiment of the present invention provides a biodegradable polymeric nanoparticles of PLA-PEG-PPG-PEG block copolymer obtained by the process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer.

Another embodiment of the present invention provides a composition comprising the biodegradable polymeric nanoparticle of PLA-PEG-PPG-PEG block copolymer obtained by the process for preparation of biodegradable polymeric nanoparticles of PLA-PEG-PPG-PEG block copolymer, wherein said process comprises (a) dissolving a PEG-PPG-PEG copolymer and poly(lactic acid) (PLA) in an organic solvent to obtain a solution (b) adding N,N,-dicyclohexylcarbodiimide (DCC) and 4-(dimethylamino) pyridine (DMAP) to the solution at a temperature in the range of −4° C. to 0° C. to obtain a reaction mixture (c) stirring the reaction mixture at 250 to 400 rpm at a temperature ranging from −4° C. to 0° C. for 20 to 28 hours to obtain the PLA-PEG-PPG-PEG block copolymer (d) dissolving the PLA-PEG-PPG-PEG block copolymer in an organic solvent and homogenizing at 250 to 400 rpm to obtain a homogenized mixture (e) adding the homogenized mixture to an aqueous phase to obtain an emulsion, and (f) stirring the emulsion at 25° C. to 30° C. at 250 to 400 rpm for 10 to 12 hours to obtain the nanoparticles of PLA-PEG-PPG-PEG block copolymer.

### Polymeric Nanoparticles Comprising Therapeutics

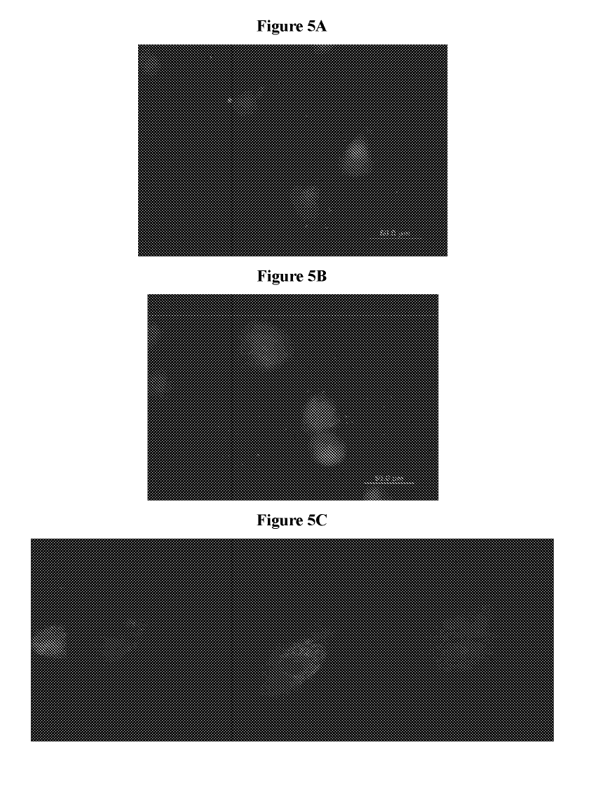

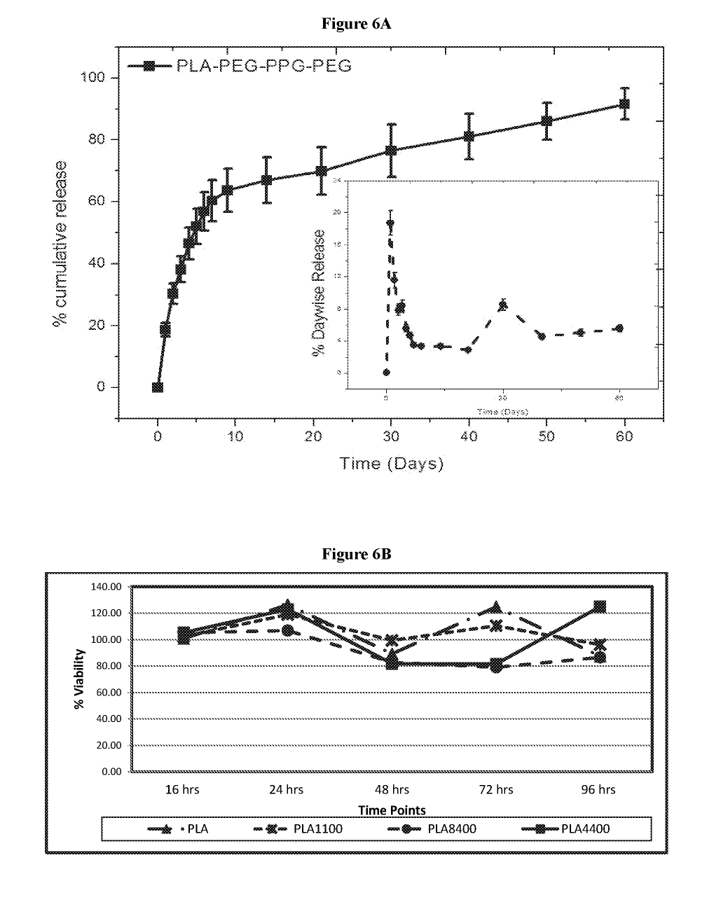

The nanoparticles of the present invention are capable of delivering active agents or entities to specific sites (FIG. 5). The particle size and release properties of the PLA-PEG-PPG-PEG nanoparticle of the present invention can be controlled by varying the molecular weight of the PLA or PEG-PPG-PEG in the polymeric matrix. The release of active agent or entity can be controlled from 12 hrs to 60 days which is an improvement over conventional PLA-PEG systems available in the art (FIG. 6A). The drug loading capacity of the nanoparticle can also be controlled by varying the average molecular weight of the block copolymer in the polymeric matrix of the nanoparticles. There is an increase in the drug loading capacity of the nanoparticle with an increase in the block length of PEG-PPG-PEG block copolymer (Table 3).

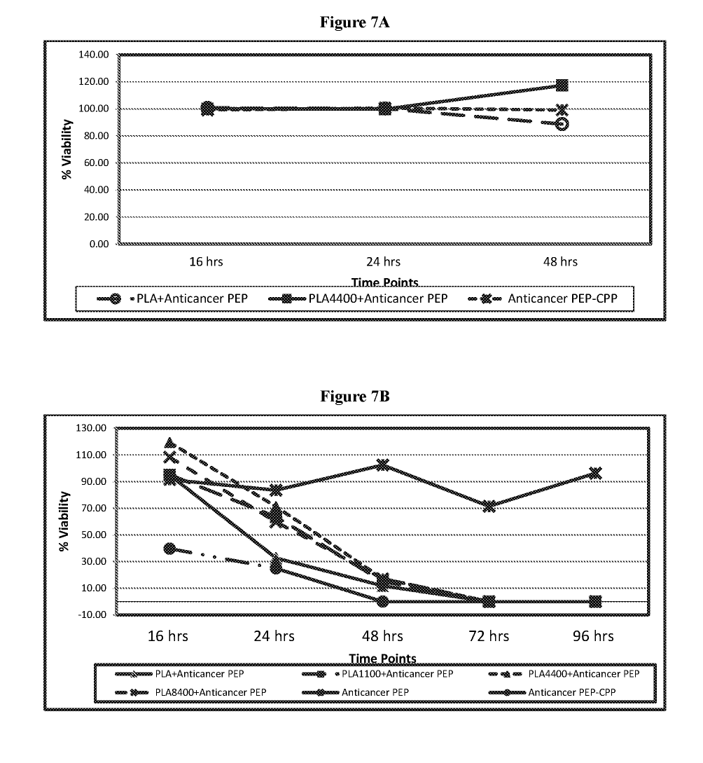

As the polymeric nanoparticles made up of PLA-PEG-PPG-PEG block copolymer are amphiphillic in nature, both hydrophobic and hydrophilic drugs can be loaded on the nanoparticles. The nanoparticles of the present invention possess high drug loading capacity due to the absence or minimal use of emulsifiers, resulting in reducing the dose load and frequency of therapeutics. The ratio of active agent or entity to nanoparticle is higher in the nanoparticles of the present invention compared to conventional systems employing emulsifiers, since the weight of the emulsifier can add up to 50% of the total formulation weight (International Journal of Pharmaceutics, 15 Jun. 2011, Volume 411, Issues 1-2, Pages 178-187; International Journal of Pharmaceutics, 2010, 387: 253-262). The nanoparticles help to achieve single and low dose drug delivery coupled with reduced toxicity. The weight percentage of the active agent to the nanocarrier system of PLA-PEG-PPG-PEG ranges from 2-20% to the nanoparticle. The higher drug loading in the nanoparticle reduces the drug dose requirement since the effective dose can be administered at a reduced dosage level. The enhanced internal loading in the polymeric nanoparticles with a prolonged activity of the loaded entities without hampering the total loading capacity of the nanoparticle leads to an effective delivery of highly potential therapeutics. FIG. 7B shows the efficacy of the anticancer peptide, L-NuBCP-9, also referred to herein as “NuBCP-9”, (L-configuration of amino acid sequence FSRSLHSLL) loaded into a nanoparticle formulation compared to the free peptide drug formulation and the conventional cell-penetrating peptide conjugated drug formulation in Primary HUVEC cell lines.

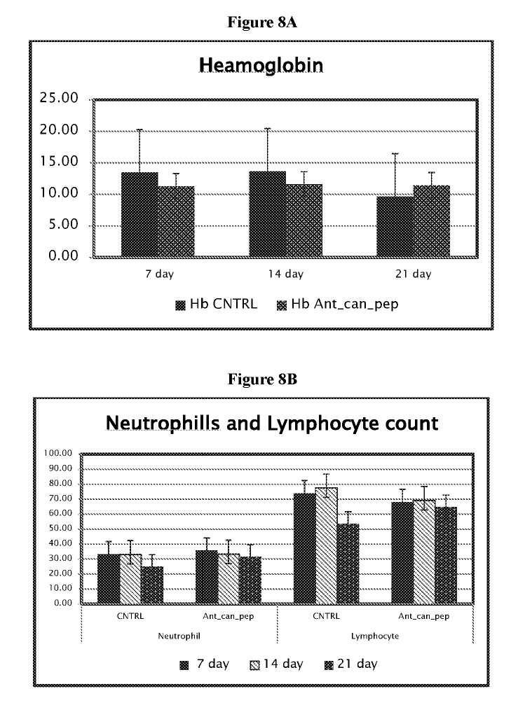

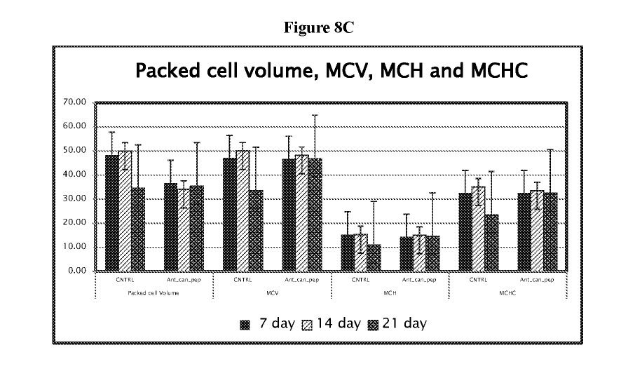

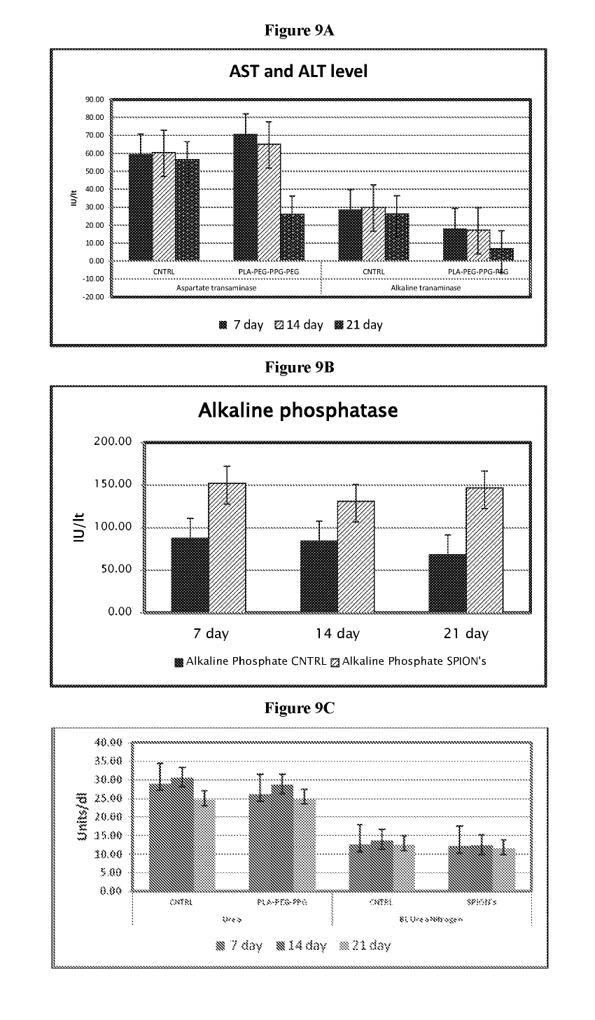

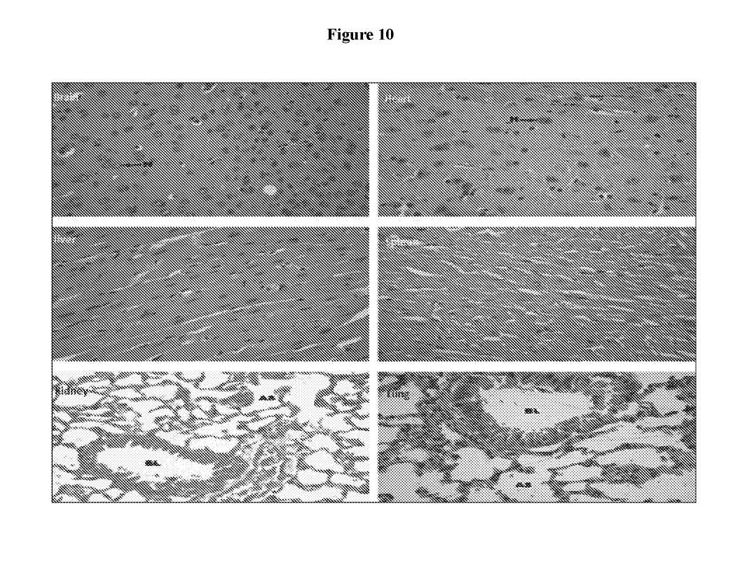

The PLA-PEG-PPG-PEG nanoparticles of the present invention are nontoxic as confirmed by in-vitro cell line studies and in-vivo mouse model studies. Hematological parameters assessed in mice treated with PLA-PEG-PPG-PEG nanoparticles at a dose of 150 mg/kg body weight showed no significant change in the complete blood count, red blood count, white blood count, neutrophil and lymphocyte levels with the control group (FIG. 8). Biochemical parameters assessed for liver and kidney functions showed no significant change in the total protein, albumin and globulin levels between the control and the nanoparticle-treated groups. The levels of the liver enzymes, alanine transaminase (ALT), aspartate transaminase (AST) and alkaline phosphatase (ALP) were non-significantly increased in the PLA-PEG-PPG-PEG nanoparticle treated group compared to control group, as seen in FIGS. 9A and 9B. There is no significant change in the levels of urea and blood urea nitrogen (BUN) in mice treated with PLA-PEG-PPG-PEG nanoparticles compared with control (FIG. 9C). The histopathology of the organs, brain, heart, liver, spleen, kidney and lung of mice injected with PLA-PEG-PPG-PEG nanoparticles is shown in FIG. 10.

The nanoparticles of the present invention can encapsulate and/or adsorb one or more entities. The entity can also be conjugated to directly to the block copolymer of the biodegradable nanoparticle. Entities of the present invention include but are not limited to, small organic molecules, nucleic acids, polynucleotides, oligonucleotides, nucleosides, DNA, RNA, SiRNA, amino acids, peptides, protein, amines, antibodies and variants thereof, antibiotics, low molecular weight molecules, chemotherapeutics, drugs or therapeutic agents, metal ions, dyes, radioisotope, contrast agent, and/or imaging agents.

Suitable molecules that can be encapsulated are therapeutic agents. Included in therapeutic agents are proteins or peptides or fragments thereof, insulin, etc., hydrophobic drugs like doxorubcin, paclitaxil, gemcetabin, docetaxel etc; antibiotics like amphotericin B, isoniazid (INH) etc, and nucleic acids. Therapeutic agents also include chemotherapeutics such as paclitaxel, doxorubicin pimozide, perimethamine, indenoisoquinolines, or nor-indenoisoquinolines.

The therapeutic agent can comprise natural and non-natural (synthetic) amino acids. Non-limiting examples include bicyclic compounds and peptidomimetics such as cyclic peptidomimetics.

It is known that the L-form or L-configuration of the therapeutic peptides are economically cheaper to manufacture but have a disadvantage in drug applications since they are known to degrade very fast in the in-vivo system compared to their D-forms. However, encapsulation of such L-peptides by the nanoparticles of the present invention does not result in degradation in circulation due to encapsulation in the core of the nanoparticles as confirmed by in-vivo studies (FIGS. 11, 12 and 13).

Targeted delivery of the nanoparticles loaded with anticancer drugs can be achieved compared to the free drug formulations prevalent in the art. The nanoparticles of the present invention can also be surface conjugated, bioconjugated, or adsorbed with one or more entities including targeting moieties on the surface of nanoparticles. Targeting moieties cause nanoparticles to localize onto a tumor or a disease site and release a therapeutic agent. The targeting moiety can bind to or associate with a linker molecules. Targeting molecules include but are not limited to antibody molecules, growth receptor ligands, vitamins, peptides, haptens, aptamers, and other targeting molecules known to those skilled in the art. Drug molecules and imaging molecules can also be attached to the targeting moieties on the surface of the nanoparticles directly or via linker molecules.

Specific, non-limiting examples of targeting moieties include vitamins, ligands, amines, peptide fragments, antibodies, aptamers, a transferrin, an antibody or fragment thereof, sialyl Lewis X antigen, hyaluronic acid, mannose derivatives, glucose derivatives, cell specific lectins, galaptin, galectin, lactosylceramide, a steroid derivative, an RGD sequence, EGF, EGF-binding peptide, urokinase receptor binding peptide, a thrombospondin-derived peptide, an albumin derivative and/or a molecule derived from combinatorial chemistry.

Further, the nanoparticles of the present invention may be surface functionalized and/or conjugated to other molecules of interest. Small low molecular weight molecules like folic acid, prostate membrane specific antigen (PSMA), antibodies, aptamers, molecules that bind to receptors or antigens on the cell surface etc., can be covalently bound to the block copolymer PEG-PPG-PEG or the PEG component of the polymeric matrix. In suitable embodiment of the present invention, the matrix comprises of polymer and an entity. In some cases the entity or targeting moiety can be covalently associated with surface of polymeric matrix. Therapeutic agents can be associated with the surface of the polymeric matrix or encapsulated throughout the polymeric matrix of the nanoparticles. Cellular uptake of the conjugated nanoparticle is higher compared to plain nanoparticles.

The nanoparticle of the present invention can comprise one or more agents attached to the surface of nanoparticle via methods well known in the art and also encapsulate one or more agents to function as a multifunctional nanoparticle. The nanoparticles of the present invention can function as multi-functional nanoparticles that can combine tumor targeting, tumor therapy and tumor imaging in an all-in-one system, providing a useful multi-modal approach in the battle against cancer. The multifunctional nanoparticle can have one or more active agents with similar or different mechanisms of actions, similar or different sites of action; or similar and different functions.

Entity encapsulation in the PLA-PEG-PPG-PEG nanoparticle is prepared by emulsion precipitation method. The PLA-PEG-PPG-PEG polymeric nanoparticle prepared using the process of the present invention is dissolved in an organic solvent comprising an organic solvent. The entity is added to the polymeric solution in the weight range of 10-20% weight of the polymer. The polymeric solution is then added drop-wise to the aqueous phase and stirred at room temperature for 10-12 hours to allow for solvent evaporation and nanoparticle stabilization. The entity-loaded nanoparticles are collected by centrifugation, dried, and stored at 2° C.-8° C. until further use. Other additives like sugars, amino acids, methyl cellulose etc., may be added to the aqueous phase in the process for the preparation of the entity-loaded polymeric nanoparticles.

The entity-loading capacity of the nanoparticles of the present invention is high, reaching nearly about 70-90% as shown in Table 3. The PLA-PEG-PPG-PEG based nanocarrier system of the present invention prevents premature degradation and effective and targeted delivery of anticancer peptide to the cancer cells. Surface foliated biodegradable PLA-PEG-PPG-PEG nanoparticles encapsulating therapeutic peptides such as NuBCP-9, Bax BH3 etc., in the core can be effectively delivered into the cytosol of the cancer cells without the use of any cell penetrating peptides. In-vitro studies with MCF-7 cell lines challenged with NuBCP-9-loaded nanoparticles showed complete killing of cells in 48-72 hrs as assessed by XTT assay (FIG. 7B) and in-vivo studies (FIGS. 11 and 12). FIG. 7B also shows the efficacy of the nanoparticles for sustained release and efficient delivery of drug compared with free drug formulations in the MCF-7 cell lines.

In suitable embodiments, higher loading of the entity in the PLA-PEG-PPG-PEG nanoparticles is achieved by linking the active agent with low molecular weight PLA. The entity is covalently linked with low molecular weight PLA by a reaction with a carbodiimide coupling reagent in combination with a hydroxyderivative. As an example, the carbodiimide coupling agent is ethyl-dimethyl aminopropylcarbodiimide and the hydroxyderivative is N-hydroxy-succinimide (EDC/NHS) chemistry. The molecular weight of PLA is in the range of about 2,000-10,000 g/mol. Higher loading of both hydrophobic and hydrophilic drugs in the PLA-PEG-PPG-PEG nanoparticles are achieved (Example 5, Tables 4 and 5). The nanoparticles with encapsulated PLA-drugs were delivered into the cytosol without the aid of cell penetrating peptides (CPPs).

Thus, provided herein is a process for preparing biodegradable polymeric nanoparticles of PLA-PEG-PPG-PEG block copolymer comprising one or more entities (e.g., one or more therapeutic agents).

In an embodiment, provided herein is a process for preparing biodegradable polymeric nanoparticles of PLA-PEG-PPG-PEG block copolymer comprising one or more entities (e.g., one or more therapeutic agents), wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity.

In another embodiment of the present invention there is provided a process for preparing biodegradable polymeric nanoparticles of PLA-PEG-PPG-PEG block copolymer comprising at least one entity, wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein said process optionally comprises the steps of washing the nanoparticles of PLA-PEG-PPG-PEG block copolymer comprising the entity with water and drying the nanoparticles by conventional method.

In another embodiment of the present invention there is provided a process for preparing biodegradable polymeric nanoparticles of PLA-PEG-PPG-PEG block copolymer comprising at least one entity, wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein the entity is selected from a group consisting of small organic molecules, nucleic acids, polynucleotides, oligonucleotides, nucleosides, DNA, RNA, amino acids, peptides, protein, antibiotics, low molecular weight molecules, pharmacologically active molecules, drugs, metal ions, dyes, radioisotopes, contrast agents imaging agents, and targeting moiety.

In another embodiment of the present invention there is provided a process for preparing biodegradable polymeric nanoparticles of PLA-PEG-PPG-PEG block copolymer comprising at least one entity, wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein the entity is a targeting moiety selected from the group consisting of vitamins, ligands, amines, peptide fragment, antibodies and aptamers.

In another embodiment of the present invention there is provided a process for preparing biodegradable polymeric nanoparticles of PLA-PEG-PPG-PEG block copolymer comprising at least one entity, wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein the entity is linked to PLA.

In another embodiment of the present invention there is provided a process for preparing biodegradable polymeric nanoparticles of PLA-PEG-PPG-PEG block copolymer comprising at least one entity, wherein said process comprises (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein the entity is linked to PLA of molecular weight in the range of 2,000 g/mol to 10,000 g/mol.

Another embodiment of the present invention provides a biodegradable polymeric nanoparticle of PLA-PEG-PPG-PEG comprising at least one entity obtained by the process comprising (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity.

Another embodiment of the present invention provides a composition comprising the biodegradable polymeric nanoparticle of PLA-PEG-PPG-PEG comprising at least one entity obtained by the process comprising (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity.

In another embodiment of the present invention, there is provided a composition comprising the biodegradable polymeric nanoparticle of PLA-PEG-PPG-PEG comprising at least one entity obtained by the process comprising (a) homogenizing the entity with the polymeric nanoparticles of PLA-PEG-PPG-PEG block copolymer dissolved in an organic solvent at 250 to 400 rpm to obtain a primary emulsion (b) emulsifying the primary emulsion in an aqueous phase at 250 to 400 rpm to obtain a secondary emulsion, and (c) stirring the secondary emulsion at 25° C. to 30° C. at 250 to 400 rpm for 10 to 12 hours to obtain the nanoparticle of PLA-PEG-PPG-PEG comprising the entity, wherein the composition optionally comprises at least one pharmaceutical excipient selected from the group consisting of preservative, antioxidant, thickening agent, chelating agent, isotonic agent, flavoring agent, sweetening agent, colorant, solubilizer, dye, flavors, binder, emollient, fillers, lubricants and preservative.

### Polymeric Nanoparticles Comprising Pharmaceutical Combinations

The biodegradable polymeric nanoparticles described herein can be used to deliver pharmaceutical combinations. For example, a pharmaceutical combination that can be delivered by the nanoparticles disclosed herein comprises a chemotherapeutic drug, e.g., paclitaxel, and an anticancer peptide, e.g., a peptide comprising NuBCP-9 (SEQ ID NO: 1) or a peptide comprising MUC1 (SEQ ID NO: 2). When delivered via a nanoparticle, the in vitro activity of paclitaxel and NuBCP-9 against breast cancer cell lines was synergistically increased, as was their activity in a EAT model in BALB/c mice (See, e.g., Example 8). The results showed approximate 40 fold decrease in IC₅₀ of paclitaxel when co-delivered with NuBCP-9 as compared to single drug alone (See, e.g., Example 8 and Table 8). The mechanism of the PTX/NuBCP-9 combination was found to involve enhanced apoptosis, which seemed to be caspase-dependent and involved in intrinsic parts of the caspase cascade in MCF7 cells. Combined application of NuBCP-9 and PTX at low concentrations was significantly more effective than either drug alone against EAT tumor model Balb/c mice. The co-delivery of paclitaxel along with NuBCP-9 anti-cancer peptide therefore may be used to effectively treat cancers such as breast cancer.

In an aspect, provided herein is a polymeric nanoparticle comprising a poly(lactic acid)-poly(ethylene glycol)-poly(propylene glycol)-poly(ethylene glycol) (PLA-PEG-PPG-PEG) tetra block copolymer, wherein the polymeric nanoparticle is loaded with

a) one or more chemotherapeutic agents; and

b) a peptide comprising NuBCP-9 (SEQ ID NO: 1) or a peptide comprising MUC1 (SEQ ID NO: 2).

In an embodiment, the polymeric nanoparticle is loaded with a peptide comprising NuBCP-9 (SEQ ID NO: 1).

In another embodiment, the polymeric nanoparticle is loaded with a peptide comprising MUC1 (SEQ ID NO: 2).

In an embodiment, the molecular weight of the PLA is between about 2,000 and about 80,000 daltons.

In another embodiment, the PLA-PEG-PPG-PEG tetra block copolymer is formed from chemical conjugation of PEG-PPG-PEG tri-block copolymer with PLA, and wherein the PEG-PPG-PEG tri-block copolymer can be of different molecular weights.

In an embodiment, the polymeric nanoparticle is loaded with

a) a chemotherapeutic agent or a targeted anti-cancer agent; and

b) a peptide comprising NuBCP-9 (SEQ ID NO: 1) or a peptide comprising MUC1 (SEQ ID NO: 2).

In an embodiment, the polymeric nanoparticle is loaded with a peptide comprising NuBCP-9 (SEQ ID NO: 1).

In another embodiment, the polymeric nanoparticle is loaded with a peptide comprising MUC1 (SEQ ID NO: 2).

In an embodiment, the chemotherapeutic agent is paclitaxel. In a further embodiment, the polymeric nanoparticle is loaded with paclitaxel and a peptide comprising NuBCP-9 in a ratio of about 9:1, 8:2, 7:3, 6:4, 5:5, 4:6, 3:7, 2:8, or 1:9.

In another embodiment, the chemotherapeutic agent is gemcitabine. In a further embodiment, the polymeric nanoparticle is loaded with gemcitabine and a peptide comprising NuBCP-9 in a ratio of about 9:1, 8:2, 7:3, 6:4, 5:5, 4:6, 3:7, 2:8, or 1:9.

In other embodiments, the chemotherapeutic agent or targeted anti-cancer agent is selected from the group consisting of doxorubicin, daunorubicin, decitabine, irinotecan, SN-38, cytarabine, docetaxel, triptolide (a diterpinoid epoxide), geldanamycin (a HSP90 inhibitor), 17-AAG, 5-FU, oxaliplatin, carboplatin, taxotere, methotrexate, and bortezomib.

In an embodiment, the polymeric nanoparticle consists essentially of a poly(lactic acid)-poly(ethylene glycol)-poly(propylene glycol)-poly(ethylene glycol) (PLA-PEG-PPG-PEG) tetra block copolymer.

In another aspect, provided herein is a polymeric nanoparticle comprising

a) a poly(lactic acid)-poly(ethylene glycol)-poly(propylene glycol)-poly(ethylene glycol) (PLA-PEG-PPG-PEG) tetra block copolymer;

b) one or more therapeutics; and

c) a peptide comprising NuBCP-9 (SEQ ID NO: 1) or a peptide comprising MUC1 (SEQ ID NO: 2),

for use in treating a disease selected from the group consisting of an autoimmune disease, an inflammatory disease, a metabolic disorder, a developmental disorder, a cardiovascular disease, a liver disease, an intestinal disease, an infectious disease, an endocrine disease and a neurological disorder.

In an embodiment, the polymeric nanoparticle consists essentially of a poly(lactic acid)-poly(ethylene glycol)-poly(propylene glycol)-poly(ethylene glycol) (PLA-PEG-PPG-PEG) tetra block copolymer.

### Compositions

In an aspect, provided herein is a polymeric nanoparticle of the invention comprising a pharmaceutical combination for use in the preparation of a medicament for the treatment or prevention of a disease such as cancer. In an embodiment, the polymeric nanoparticle comprising the pharmaceutical combination is for use in the preparation of a medicament for the treatment of cancer.

In another aspect, the present invention provides for the use of the biodegradable polymeric nanoparticle consisting essentially of PLA-PEG-PPG-PEG block copolymer comprising a pharmaceutical combination for the manufacture of a medicament.

Also provided herein is a composition comprising the polymeric nanoparticle of the invention, wherein the polymeric nanoparticle comprises a pharmaceutical combination of therapeutic agents (e.g., a peptide comprising NuBCP-9 and a chemotherapeutic agent or a targeted anti-cancer agent) and a pharmaceutically acceptable carrier.

In an aspect, provided herein is use of polymeric nanoparticle comprising a pharmaceutical combination for the manufacture of a medicament for the treatment or prevention of a disease, such as cancer. In an embodiment, the use of a polymeric nanoparticle comprising a pharmaceutical combination is for the manufacture of a medicament for the treatment of a disease such as cancer.

In an embodiment of the compositions provided herein, the polymeric nanoparticle further comprises a targeting moiety attached to the outside of the polymeric nanoparticle, and wherein the targeting moiety is an antibody, peptide, or aptamer.

In an aspect, provided herein is a composition comprising

a) polymeric nanoparticles comprising a poly(lactic acid)-poly(ethylene glycol)-poly(propylene glycol)-poly(ethylene glycol) (PLA-PEG-PPG-PEG) tetra block copolymer;

b) one or more chemotherapeutic agents or anti-cancer targeting agents; and

c) a peptide comprising NuBCP-9 (SEQ ID NO: 1) or a peptide comprising MUC1 (SEQ ID NO: 2).

In an embodiment of the composition, the composition comprises a peptide comprising NuBCP-9 (SEQ ID NO: 1).

In another embodiment of the composition, the composition comprises a peptide comprising MUC1 (SEQ ID NO: 2).

In an embodiment of the composition, the molecular weight of PLA is between about 2,000 and about 80,000 daltons.

In an embodiment of the composition, the PLA-PEG-PPG-PEG tetra block copolymer is formed from chemical conjugation of PEG-PPG-PEG tri-block copolymer with PLA, and wherein the PEG-PPG-PEG tri-block copolymer can be of different molecular weights.

In an embodiment of the composition, the polymeric nanoparticles are loaded with

a) a chemotherapeutic agent or a targeted anti-cancer agent; and

b) a peptide comprising NuBCP-9 (SEQ ID NO: 1) or a peptide comprising MUC1 (SEQ ID NO: 2).

In an embodiment, the polymeric nanoparticle is loaded with a peptide comprising NuBCP-9 (SEQ ID NO: 1).

In another embodiment, the polymeric nanoparticle is loaded with a peptide comprising MUC1 (SEQ ID NO: 2).

In a further embodiment of the composition, the chemotherapeutic agent is paclitaxel.

In yet a further embodiment of the composition, the polymeric nanoparticles are loaded with paclitaxel and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the composition, the chemotherapeutic agent is gemcitabine. In a further embodiment of the composition, the polymeric nanoparticles are loaded with gemcitabine and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the composition, the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

In another aspect, provided herein is a pharmaceutical composition comprising

a) polymeric nanoparticles comprising a poly(lactic acid)-poly(ethylene glycol)-poly(propylene glycol)-poly(ethylene glycol) (PLA-PEG-PPG-PEG) tetra block copolymer;

b) one or more therapeutic agents; and

c) a peptide comprising NuBCP-9 (SEQ ID NO: 1),

for use in treating a disease selected from the group consisting of cancer, an autoimmune disease, an inflammatory disease, a metabolic disorder, a developmental disorder, a cardiovascular disease, liver disease, an intestinal disease, an infectious disease, an endocrine disease and a neurological disorder.

In an embodiment, the composition is for use in treating cancer. In a further embodiment, the cancer is breast cancer, prostate cancer, non-small cell lung cancer, metastatic colon cancer, pancreatic cancer, or a hematological malignancy. In yet a further embodiment, the cancer is breast cancer.

In another aspect, provided herein is a pharmaceutical composition comprising

a) polymeric nanoparticles comprising a poly(lactic acid)-poly(ethylene glycol)-poly(propylene glycol)-poly(ethylene glycol) (PLA-PEG-PPG-PEG) tetra block copolymer;

b) one or more therapeutic agents; and

c) a peptide comprising MUC1 (SEQ ID NO: 2),

for use in treating a disease selected from the group consisting of cancer, an autoimmune disease, an inflammatory disease, a metabolic disorder, a developmental disorder, a cardiovascular disease, liver disease, an intestinal disease, an infectious disease, an endocrine disease and a neurological disorder.

In an embodiment, the composition is for use in treating cancer. In a further embodiment, the cancer is breast cancer, prostate cancer, non-small cell lung cancer, metastatic colon cancer, pancreatic cancer, or a hematological malignancy. In yet a further embodiment, the cancer is breast cancer.

In an embodiment of any of the compositions provided herein, the polymeric nanoparticles consist essentially of poly(lactic acid)-poly(ethylene glycol)-poly(propylene glycol)-poly(ethylene glycol) (PLA-PEG-PPG-PEG) tetra block copolymer.

In an embodiment of any of the compositions provided herein, the polymeric nanoparticles further comprise a targeting moiety attached to the outside of the polymeric nanoparticles, and wherein the targeting moiety is an antibody, peptide, or aptamer.

Suitable pharmaceutical compositions or formulations can contain, for example, from about 0.1% to about 99.9%, preferably from about 1% to about 60%, of the active ingredient(s). Pharmaceutical formulations for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

The pharmaceutical compositions can contain, as the active ingredient, one or more of the nanoparticles of the invention in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Some examples of suitable excipients include lactose (e.g. lactose monohydrate), dextrose, sucrose, sorbitol, mannitol, starches (e.g. sodium starch glycolate), gum acacia, calcium phosphate, alginates, tragacanth, gelatin, calcium silicate, colloidal silicon dioxide, microcrystalline cellulose, polyvinylpyrrolidone (e.g. povidone), cellulose, water, syrup, methyl cellulose, and hydroxypropyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

### Methods for Treating

In yet another aspect, the present invention provides a method for treating disease comprising administering biodegradable polymeric nanoparticles of the inventions (e.g., consisting essentially of PLA-PEG-PPG-PEG) comprising a pharmaceutical combination (i.e., more than one therapeutic agent) to a subject in need thereof.

In an embodiment, the disease is selected from the group consisting of cancer, an autoimmune disease, an inflammatory disease, a metabolic disorder, a developmental disorder, a cardiovascular disease, a liver disease, an intestinal disease, an infectious disease, an endocrine disease and a neurological disorder.

Also provided herein is a method for treating cancer in a subject in need thereof comprising administering to the subject a therapeutically effective amount of a polymeric nanoparticle comprising a PLA-PEG-PPG-PEG tetra block copolymer loaded with

a) a chemotherapeutic agent and/or a targeted anti-cancer agent; and

b) a peptide comprising NuBCP-9 (SEQ ID NO: 1) or a peptide comprising MUC1 (SEQ ID NO:2).

In an embodiment, the polymeric nanoparticle is loaded with a peptide comprising NuBCP-9 (SEQ ID NO: 1).

In another embodiment, the polymeric nanoparticle is loaded with a peptide comprising MUC1 (SEQ ID NO: 2).

In an embodiment, the chemotherapeutic agent is paclitaxel. In a further embodiment, the polymeric nanoparticle is loaded with paclitaxel and a peptide comprising NuBCP-9 in a ratio of about 9:1, 8:2, 7:3, 6:4, 5:5, 4:6, 3:7, 2:8, or 1:9.

In another embodiment, the chemotherapeutic agent is gemcitabine. In a further embodiment, the polymeric nanoparticle is loaded with gemcitabine and a peptide comprising NuBCP-9 in a ratio of about 9:1, 8:2, 7:3, 6:4, 5:5, 4:6, 3:7, 2:8, or 1:9.

In other embodiments, the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib. In a further embodiment, the polymeric nanoparticle is loaded with the chemotherapeutic or targeted anti-cancer agent (e.g., doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, or bortezomib) and a peptide comprising NuBCP-9 in a ratio of about 9:1, 8:2, 7:3, 6:4, 5:5, 4:6, 3:7, 2:8, or 1:9.

In an embodiment, the cancer is wherein the cancer is breast cancer, prostate cancer, non-small cell lung cancer, metastatic colon cancer, pancreatic cancer, or a hematological malignancy. In a particular embodiment, the cancer is breast cancer.

In an aspect, provided herein is method for treating a disease in a subject in need thereof comprising administering to the subject a therapeutically effective amount of a polymeric nanoparticle consisting essentially of a PLA-PEG-PPG-PEG tetra block copolymer, wherein the polymeric nanoparticle is loaded with

a) one or more therapeutic agents; and

b) a peptide comprising NuBCP-9 (SEQ ID NO: 1) or a peptide comprising MUC1 (SEQ ID NO: 2).

In an embodiment, the polymeric nanoparticle is loaded with a peptide comprising NuBCP-9 (SEQ ID NO: 1).

In another embodiment, the polymeric nanoparticle is loaded with a peptide comprising MUC1 (SEQ ID NO: 2).

In an embodiment, the disease selected from the group consisting of cancer, an autoimmune disease, an inflammatory disease, a metabolic disorder, a developmental disorder, a cardiovascular disease, a liver disease, an intestinal disease, an infectious disease, an endocrine disease and a neurological disorder.

In another aspect, provided herein is a method for treating cancer in a subject in need thereof comprising administering to the subject a therapeutically effective amount of a pharmaceutical composition comprising

a) polymeric nanoparticles comprising a PLA-PEG-PPG-PEG tetra block copolymer;

b) a chemotherapeutic agent and/or an anti-cancer targeted agent; and

c) a peptide comprising NuBCP-9 (SEQ ID NO: 1) or a peptide comprising MUC1 (SEQ ID NO: 2).

In an embodiment of the method, the pharmaceutical composition comprises a peptide comprising NuBCP-9 (SEQ ID NO: 1).

In another embodiment of the method, the pharmaceutical composition comprises a peptide comprising MUC1 (SEQ ID NO: 2).

In an embodiment of the method, the chemotherapeutic agent is paclitaxel. In a further embodiment of the method, the polymeric nanoparticles are loaded with paclitaxel and a peptide comprising NuBCP-9 (SEQ ID NO: 1) in a ratio of about 9:1, 8:2, 7:3, 6:4, 5:5, 4:6, 3:7, 2:8, or 1:9.

In another embodiment of the method, the chemotherapeutic agent is gemcitabine. In a further embodiment of the method, the polymeric nanoparticles are loaded with gemcitabine and a peptide comprising NuBCP-9 in a ratio of about 9:1, 8:2, 7:3, 6:4, 5.5, 4-6, 3:7, 2:8, or 1:9.

In another embodiment of the method, the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

In an embodiment of the method, the cancer is breast cancer, prostate cancer, non-small cell lung cancer, metastatic colon cancer, pancreatic cancer, or a hematological malignancy.

The administration of a polymeric nanoparticle comprising a pharmaceutical combination may result not only in a beneficial effect, e.g. a synergistic therapeutic effect, e.g. with regard to alleviating, delaying progression of or inhibiting the symptoms, but also in further surprising beneficial effects, e.g. fewer side-effects, more durable response, an improved quality of life or a decreased morbidity, compared with a monotherapy (either monotherapy using the polymeric nanoparticle delivery system, or monotherapy where the agent is delivered by conventional means) applying only one of the pharmaceutically therapeutic agents used in the combination of the invention.

It can be shown by established test models that a polymeric nanoparticle comprising a pharmaceutical combination results in the beneficial effects described herein before. The person skilled in the art is fully enabled to select a relevant test model to prove such beneficial effects. The pharmacological activity of a polymeric nanoparticle comprising a pharmaceutical combination may, for example, be demonstrated in a clinical study or in an animal model.

In determining a synergistic interaction between one or more components, the optimum range for the effect and absolute dose ranges of each component for the effect may be definitively measured by administration of the components over different w/w ratio ranges and doses to subjects in need of treatment. For humans, the complexity and cost of carrying out clinical studies on patients may render impractical the use of this form of testing as a primary model for synergy. However, the observation of synergy in certain experiments (see, e.g., Example 8) can be predictive of the effect in other species, and animal models exist may be used to further quantify a synergistic effect. The results of such studies can also be used to predict effective dose ratio ranges and the absolute doses and plasma concentrations.

In an embodiment, polymeric nanoparticle comprising a pharmaceutical combination or a pharmaceutical composition comprising polymeric nanoparticles comprising a pharmaceutical combination, or both, as provided herein display a synergistic effect. The term “synergistic effect” as used herein, refers to action of two agents such as, for example, paclitaxel and a peptide comprising NuBCP-9 to produce an effect, for example, slowing the symptomatic progression of cancer or symptoms thereof, which is greater than the simple addition of the effects of each drug administered by themselves (either administered by themselves using the polymeric nanoparticle delivery system, or delivered by themselves wherein the agent is delivered by conventional means). A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the pharmaceutical combination. The corresponding graphs associated with the equations referred to above are the concentration-effect curve, isobologram curve and combination index curve, respectively.

In a further embodiment, the provided herein is a polymeric nanoparticle comprising a synergistic pharmaceutical combination for administration to a subject, wherein the dose range of each component corresponds to the synergistic ranges suggested in a suitable tumor model or clinical study.

The effective dosage of each of the combination partners employed in the combination used in forming the polymeric nanoparticles provided herein may vary depending on the particular compound or pharmaceutical composition employed, the mode of administration, the condition being treated, and the severity of the condition being treated. Thus, the dosage regimen of the polymeric nanoparticle comprising the pharmaceutical combination is selected in accordance with a variety of factors including the route of administration and the renal and hepatic function of the patient.

While certain ratios of pharmaceutical combinations are disclosed, optimum ratios, and concentrations of the combination partners (e.g., a peptide comprising NuBCP-9 and paclitaxel) used in forming the polymeric nanoparticles provided herein that yield efficacy without toxicity are based on the kinetics of the therapeutic agents' availability to target sites, and are determined using methods known to those of skill in the art.

The methods of treating disclosed herein can be particularly suited for a subject who has been diagnosed with at least one of the cancers described as treatable by the use of a polymeric nanoparticle described herein. For example, the biodegradable tetrablock polymeric nanoparticles for intracellular PTX delivery (PTX/NPs) are highly effective in inhibiting PTX efflux. As described in Example 9, PTX/NPs are active against P-gp-expressing breast cancer cells resistant to PTX and nab-paclitaxel.

In some embodiments, the subject has been diagnosed with a cancer named herein, and has proven refractory to treatment with at least one conventional chemotherapeutic agent, e.g., paclitaxel, nab-paclitaxel (ABRAXANE), docetaxel, vincristine, vinblastine, taxol. Thus, in one embodiment, the treatments of the invention are directed to subjects or patients who have received one or more than one treatment with a conventional chemotherapeutic and remain in need of more effective treatment. In a particular embodiment, the treatments of the invention are directed to subjects or patients who have received treatment with paclitaxel or nab-paclitaxel and remain in need of more effective treatment.

In an embodiment of any of the methods provided herein, the subject is resistant to treatment with paclitaxel or nab-paclitaxel.

In an embodiment of any of the methods provided herein, the subject is refractory to treatment with paclitaxel or nab-paclitaxel.

In another embodiment any of the methods provided herein, the subject is in relapse after treatment with paclitaxel or nab-paclitaxel.

In another aspect, provided herein is a method for inhibiting paclitaxel efflux in a cell comprising contacting the cell with an effective amount of polymeric nanoparticles comprising PLA-PEG-PPG-PEG tetra block copolymer.

In an embodiment of this method, the polymeric nanoparticles are loaded with paclitaxel.

In yet another aspect, provided herein is a method for blocking P-glycoprotein expression in a cell comprising contacting the cell with an effective amount of polymeric nanoparticles comprising PLA-PEG-PPG-PEG tetra block copolymer.

In another aspect, provided herein is a method for reversing P-glycoprotein-mediated drug resistance in a cell comprising contacting the cell with an effective amount of polymeric nanoparticles comprising PLA-PEG-PPG-PEG tetra block copolymer.

In an embodiment of any of the methods provided herein, the polymeric nanoparticles consist essentially of PLA-PEG-PPG-PEG tetra block copolymer.

In another aspect, provided herein is a method for causing a cancer cell having resistance against a first chemotherapeutic comprising contacting the cancer cell with polymeric nanoparticles comprising PLA-PEG-PPG-PEG tetra block copolymer, wherein the polymeric nanoparticles are loaded with a second chemotherapeutic, and wherein the resistance of the cancer cell against the first chemotherapeutic is caused by upregulation of P-glycoprotein.

In an embodiment of this method, the polymeric nanoparticles consist essentially of PLA-PEG-PPG-PEG tetra block copolymer.

In an embodiment of this method, the cancer cell is a breast cancer cell.

In an embodiment of this method, the first chemotherapeutic is paclitaxel.

In an embodiment of this method, the second chemotherapeutic is paclitaxel.

In an embodiment of this method, the polymeric nanoparticles are loaded with a peptide comprising NuBCP-9 (SEQ ID NO: 1).

In another embodiment of this method, the polymeric nanoparticles are loaded with a peptide comprising MUC1 (SEQ ID NO: 2).

Although the subject matter has been described in considerable detail with reference to certain preferred embodiments thereof, other embodiments are possible. As such, the spirit and scope of the appended claims should not be limited to the description of the preferred embodiment contained therein.

### Examples

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1: Preparation of Polymeric Nanoparticles of PLA-PEG-PPG-PEG Block Copolymer

Poly(lactic acid) (Mw. ˜45,000-72,000 g/mol), PEG-PPG-PEG (Table 1) and tissue culture reagents were obtained from Sigma-Aldrich (St. Louis, Mo.). All reagents were analytical grade or above and used as received, unless otherwise stated. Cell lines were obtained from NCCS Pune, India. NuBCP-9 peptide was custom synthesized with 95% purity.

### Preparation of PLA-PEG-PPG-PEG Block Copolymer

5 gm of poly (lactic acid) (PLA) with an average molecular weight of 60,000 g/mol was dissolved in 100 ml CH₂Cl₂ (dichloromethane) in a 250 ml round bottom flask. To this solution, 0.7 g of PEG-PPG-PEG polymer (molecular weight range of 1100-12,500 Mn) was added. The solution was stirred for 10-12 hours at 0° C. To this reaction mixture, 5 ml of 1% N,N-dicyclohexylcarbodimide (DCC) solution was added followed by slow addition of 5 ml of 0.1% 4-Dimethylaminopyridine (DMAP) at −4° C. to 0° C./sub zero temperatures. The reaction mixture was stirred for the next 24 hours followed by precipitation of the PLA-PEG-PPG-PEG block copolymer with diethyl ether and filtration using Whatman filter paper No. 1. The PLA-PEG-PPG-PEG block copolymer precipitates so obtained are dried under low vacuum and stored at 2° C. to 8° C. until further use.

### Preparation of PLA-PEG-PPG-PEG Nanoparticles

The PLA-PEG-PPG-PEG nanoparticles were prepared by emulsion precipitation method. 100 mg of the PLA-PEG-PPG-PEG copolymer obtained by the above mentioned process was separately dissolved in an organic solvent, for example, acetonitrile, dimethyl formamide (DMF) or dichloromethane to obtain a polymeric solution.

The nanoparticles were prepared by adding this polymeric solution drop wise to the aqueous phase of 20 ml distilled water. The solution was stirred magnetically at room temperature for 10 to 12 hours to allow residual solvent evaporation and stabilization of the nanoparticles. The nanoparticles were then collected by centrifugation at 25,000 rpm for 10 min and washed thrice using distilled water. The nanoparticles were further lyophilized and stored at 2° C. to 8° C. until further use.

### Characterization of Polymeric Nanoparticles of PLA-PEG-PPG-PEG Block Copolymer

The shape of the nanoparticles obtained by the process mentioned above is essentially spherical as is seen in the Transmission Electron Micrsocopy Image shown in FIGS. 4A-B. The TEM images allowed for the determination of the particle size range, which is about 30 to 120 nm. The hydrodynamic radius of the nanoparticle was measured using a dynamic light scattering (DLS) instrument and is in the range of 110-120 nm (Table 2).

The characteristics of the PLA-PEG-PPG-PEG nanoparticles synthesized using a range of molecular weights of the block copolymer, PEG-PPG-PEG, is shown in Table 2. The FTIR spectra of the PLA, PLA-PEG, the block copolymer PEG-PPG-PEG and the polymeric nanoparticles PLA-PEG-PPG-PEG are given in FIG. 2A. The FTIR proved to be insensitive to the differences between these species. Therefore, further characterization was done using NMR.

The NMR spectra of the PLA-PEG-PPG-PEG nanoparticles obtained using different molecular weights of the block copolymer, PEG-PPG-PEG, are shown in FIGS. 3A-C. In the figures, the proton with a chemical shift of about 5.1 represents the ester proton of PLA and the proton with a chemical shift at around 3.5 represent the ether proton of PEG-PPG-PEG. The presence of both the protons in the spectra confirms the conjugation of PLA with PEG-PPG-PEG.

### Example 2: Preparation of an Entity-Encapsulated Nanoparticle

### Preparation of a Drug Encapsulated Polymeric Nanoparticle

The nanoparticles of the present invention are amphiphillic in nature and are capable of being loaded with both hydrophobic drugs like Doxorubicin and hydrophilic drugs like the anticancer nine mer peptides, (L-NuBCP-9, L-configuration of FSRSLHSLL), 16 mer-BH3 domain etc.

0.100 g of the PLA-PEG-PPG-PEG nanoparticle prepared using the process of Example 1 is dissolved in 5 ml of an organic solvent like acetonitrile (CH₃CN), dimethyl formamide (DMF; C₃H₇NO), acetone or dichloromethane (CH₂Cl₂).

1-5 mg of the drug entity, NuBCP-9 (L-configuration of FSRSLHSLL), is dissolved in an aqueous solution and is added to the above polymeric solution. The entity is usually taken in the weight range of about 10-20% weight of the polymer. This solution is briefly sonicated for 10-15 seconds at 250-400 rpm produce a fine primary emulsion.

The fine primary emulsion is added drop wise using a syringe/micropipette to the aqueous phase of 20 ml distilled water and stirred magnetically at 250 to 400 rpm at 25° C. to 30° C. for 10 to 12 h in order to allow solvent evaporation and nanoparticle stabilization. The aqueous phase further comprises a sugar additive. The resulting nanoparticle suspension was allowed to stir overnight, in an open, uncovered condition to evaporate the residual organic solvent. The NuBCP-9 encapsulated polymeric nanoparticles are collected by centrifugation at 10,000 g for 10 min or by ultrafiltration at 3000 g for 15 min. (Amicon Ultra, Ultracel membrane with 100,000 NMWL, Millipore, USA). The nanoparticles are resuspended in distilled water, washed thrice and lyophilized. They are stored at 2° C. to 8° C. until further use. The polymeric nanoparticles are highly stable with no stealth character.

### Comparison of the Loading Efficacy of the Polymeric Nanoparticle Prepared Using Different Weights of the Co-Polymer

PLA-PEG-PPG-PEG polymeric nanoparticles were prepared using different molecular weights of the PEG-PPG-PEG polymer using the process as mentioned above. Pyrene loaded PLA-PEG-PPG-PEG polymeric nanoparticles were prepared using the PLA-PEG-PPG-PEG copolymer synthesized using varying molecular weights of the PEG-PPG-PEG polymer. Pyrene was taken in the range of 2-20% weight of the PLA-PEG-PPG-PEG block copolymer and fluorescent dye-loaded nanoparticles were prepared. The entity loading capacity of the nanoparticles varied depending on the molecular weight of the PEG-PPG-PEG polymer used for the synthesis of the nanoparticles. Table 3 provides the percentage of the imaging molecule encapsulated by the polymeric nanoparticles produced using different molecular weights of the block copolymer.

### Cellular Internalization of the Fluorescent Dye, Rhodamine

Rhodamine loaded PLA-PEG-PPG-PEG polymeric nanoparticles were prepared using the process as mentioned above. Rhodamine was taken in the range of 2-20% weight of the PLA-PEG-PPG-PEG block copolymer and fluorescent dye-loaded nanoparticles were prepared.

1×10⁵ MCF-7 cells were initially plated and grown to 60% confluence on cover slip flasks. Cells were then washed twice with phosphate-buffered saline (PBS) and cultured in 10 ml of DMEM medium containing 10% Foetal Bovine Serum (FBS) and 1% penicillin/streptomycin for 24 h. The growth medium was then aspirated and the cells were washed twice with PBS. The rhodamine-loaded nanoparticles were added to cells attached to coverslips and incubated at 37° C. for 12 hrs. After incubation, cells were washed, and coverslips were removed. This was followed by washing with PBS solution and finally fixed with 4% paraformaldehyde for 20 minutes at room temperature. After removing the fixing agent, the cells were washed and cells were stain with DAPI (florescent dye-stain nuclei cells) for 5 min and then rinsed in running tap water for 1 min. The coverslips were then analyzed using confocal fluorescent microscope (Olympus, Fluoview FV1000 Microscope, Japan). Cellular internalization of nanoparticles in MCF-7 cells was confirmed by using fluorescent dye (Rhodamine B) loaded nanoparticles in conjunction with Confocal Laser Scanning Microscope (CLSM) (FIG. 5).

### Example 3: Preparation of Drug Encapsulated Polymeric Nanoparticle with a Targeting Moiety

Various small molecules like amines or amino acids which provide a —COOH or —NH₂ functionality, respectively, may be used for conjugation of biomolecules as targeting moieties onto the polymeric nanoparticles of the present invention.

### Preparation of PLA-PEG-PPG-PEG-Lysine

PLA-PEG-PPG-PEG copolymer was conjugated to amino acid, lysine, to have —NH₂ group. 5 g of PLA-PEG-PPG-PEG and 0.05 g of lysine were dissolved in 100 ml acetonitrile/dichloromethane (1:1) in 250 ml RB flask and allowed to stir at −4-0° C. To this solution, 1% N,N-Dicyclohexylcarbodimide (DCC) solution was added followed by slow addition of 0.1% 4-Dimethylaminopyridine (DMAP) at 0° C. The reaction mixture was stirred for 24 hours after which PLA-PEG-PPG-PEG-Lysine was precipitated by diethyl ether and filtered through Whatman filter paper No. 1. Precipitates were dried under low vacuum and kept at 2-8° C. until further use.

Preparation of Nanoparticles from PLA-PEG-PPG-PEG-Lysine

For nanoparticles preparation, PLA-PEG-PPG-PEG-Lysine copolymer (100 mg) was dissolved in acetonitrile (or dimethyl formamide (DMF) or dichloromethane). Drug (about 10-20% weight of the polymer) was then added to the solution with brief sonication of 15 s to produce a primary emulsion. The resulting primary emulsion was added drop-wise to the aqueous phase of distilled water (20 ml) and stirred magnetically at room temperature for 10-12 hrs in order to allow solvent evaporation and nanoparticle stabilization. The formed nanoparticles were collected by centrifugation at 25,000 rpm for 10 min and washed thrice using distilled water and lyophilized followed by storage at 2-8° C. for further use.

Bio-Conjugation of Nanoparticles with Folic Acid (FA)

20 mg of lyophilized PLA-PEG-PPG-PEG nanoparticles were dissolved in milliQ water and were treated with N-(3-diethylaminopropyl)-N-ethylcarbodiimide (EDC)(50 μl, 400 mM) and N-hydroxysuccinamide (NHS) (50 μl, 100 mM) and the mixture was gently shaken for 20 min. After this folic acid solution of 10 mM was added and the solution was gently shaken for 30 minutes followed by filtration using an amikon filter to remove un-reacted FA which remains in the filtrate. Folic acid conjugated nanoparticles were lyophilized followed by storage at −20° C.

### Example 4: Evaluation of the Delivery Potential of the PLA-PEG-PPG-PEG Polymeric Nanoparticle

### In-Vitro Release of Encapsulated Drug by the Polymeric Nanoparticle PLA-PEG-PPG-PEG

A mixture containing 10 ml phosphate buffer saline and 10 mg PLA-PEG-PPG-PEG nanoparticles encapsulating rhodamine B-conjugated NuBCP-9 (drug) was stirred at 200 rpm at 37° C. Supernatant samples of the mixture were collected by centrifugation at 25,000 rpm at different time intervals for a period of 6 days. The nanoparticles were re-suspended in fresh buffer after each centrifugation. 2 ml of the supernatant was subjected to protein estimation using BCA kit (Pierce, USA) to evaluate the amount of drug release spectrophotometrically at 562 nm. The drug release was calculated by means of a standard calibration curve. It was observed that the release of the drug by the PLA-PEG-PPG-PEG polymeric nanoparticles can be controlled better than the conventional PLA nanoparticles (FIG. 6A).

### The XTT (sodium 2,3,-bis(2-methoxy-4-nitro-5-sulfophenyl)-5-[(phenylamino)-carbonyl]-2H-tetrazolium Inner Salt) Assay

Cell viability using XTT (sodium 2,3,-bis(2-methoxy-4-nitro-5-sulfophenyl)-5-[(phenylamino)-carbonyl]-2H-tetrazolium inner salt) assays were carried out in Primary HUVEC cell lines and the MCF-7 cell lines (FIGS. 6B, 7A and 7B).

A total of 1×10⁴ MCF-7 cells were seeded on each well of a 96-well plate and cultured for 24 h. After 24 hours, cells in each plate were treated with polymeric nanoparticles of the present invention containing 5 M NuBCP-9 peptide or control nanoparticles without any peptide. Cells were also separately treated with the same concentration of NuBCP-9 peptide without any cell penetrating peptide (CPP). The cells were incubated with the nanoparticles for different intervals of time ranging from 16 h, 24 h, 48 h, 72 h and 96 h. After incubation, the medium containing PLA-PEG-PPG-PEG nanoparticles loaded with anticancer peptide-NuBCP-9 was exchanged with fresh medium, and 10 μl of the reconstitute XTT mixture kit reagent were added to each well. After culturing for 4 h, the absorbance of the sample was measured by using a microtiter plate reader (Bio-Rad, CA, U.S.A.) at 450 nm. The proliferation of cells was determined as the percentage of viable cells of the untreated control and analyzed in triplicate. FIG. 6B shows the effect of NuBCP-9-loaded PLA-PEG-PPG-PEG nanoparticle on the cell viability of MCF-7 cell line in relation to time. FIG. 7A shows the effect of the drug NuBCP-9 loaded PLA-PEG-PPG-PEG nanoparticle on the cell viability of Primary HUVEC cell line in relation to time.

### Example 5: Modification of Peptide Drugs to Achieve Higher Therapeutic Loading in Nanoparticles

Higher loading of hydrophobic as well as hydrophilic therapeutic agents was achieved by covalently modifying the drug moiety with low molecular weight PLA. The peptide drug is modified using low molecular weight of PLA using ethyl-dimethyl aminopropylcarbodiimide and N-hydroxy-succinimide (EDC/NHS) chemistry. The average molecular weight of the PLA used for linking the entity is usually in the range of about 2,000-10,000 g/mol.

1 g of PLA having molecular weight of 5,000 g/mol was dissolved in 10 ml acetonitrile. To this solution, 500 μl of N-(3-diethylaminopropyl)-N-ethylcarbodimide (EDC; 400 mM) in dichloromethane and 500 μl N-hydroxysuccinamide (NHS; 100 mM) in dichloromethane was added. The mixture was gently shaken for 2 hours followed by precipitation of PLA with diethyl ether. This PLA was termed “activated” PLA. 1 mmol of activated PLA was dissolved in acetonitrile and to this solution, 1 mmol of peptide drug NuBCP-9, was added and the reaction mixture was gently shaken again for 30 min. This mixture was then precipitated with diethyl ether and dried under low vacuum followed by storage at −20° C. until further use.

The drug loading capacity of the polymeric nanoparticle increased with an increase in the weight of the block copolymer used for the preparation of the nanoparticle. The drug loading capacity of the nanoparticle is also significantly increased by the conjugation of the low molecular weight PLA with the therapeutic agent (i.e. NuBCP-9) prior to the loading of the drug into the polymeric nanoparticles, as shown in Tables 4 and 5. The increase in the drug loading capacity of the nanoparticles of the present invention is by 5% to 10%.

### Example 6: In Vivo Studies to Evaluate the Safety and Toxicity of the Nanoparticles

Studies were conducted in BALB/c mice to evaluate the toxicity and safety of the PLA-PEG-PPG-PEG polymeric nanoparticles prepared using the process as given in Example 1.

### Hematology Parameters

PLA-PEG-PPG-PEG nanoparticles were intravenously injected in the animal group at a single dose of 150 mg/kg body weight and hematology parameters were evaluated in the control and nanoparticle-treated groups at intervals of 7 days for a period of 21 days. The control group received no nanoparticles.

There was no significant change in the Complete Blood Count (CBC), Red blood cell (RBC) count, White blood cell (WBC) count, Neutrophils, lymphocytes, packed cell volume, MCV (Mean Corpuscular Volume), MCH (Mean Corpuscular Hemoglobin) and MCHC (Mean Corpuscular Hemoglobin Concentration) between the control and the nanoparticle-treated groups as seen in FIG. 8.

### Biochemistry Blood Assays for Liver and Kidney Functions

PLA-PEG-PPG-PEG nanoparticles were intravenously injected in the animal group at a single dose of 150 mg/kg body weight and hematology parameters were evaluated in the control and nanoparticle-treated groups at intervals of 7 days for a period of 21 days.

There were no significant changes in the total protein, albumin and globulin levels between the control and the treated groups. The levels of the liver enzymes, alanine transaminase (ALT), aspartate transaminase (AST) and alkaline phosphatase (ALP) were non-significantly increased in the PLA-PEG-PPG-PEG nanoparticle treated group as seen in FIG. 9. Urea and Blood urea nitrogen (BUN) is a good indicator of renal function. There was no significant change in the urea and BUN levels of treated mice compared to control as seen in FIG. 9.

Histopathology of the Organs of Mice Treated with PLA-PEG-PPG-PEG Nanoparticles

BALB/c mice were treated with PLA-PEG-PPG-PEG nanoparticles at a single dose of 150 mg/kg body weight. After 21 days, the animals were sacrificed and histology of the organ tissues was carried out to assess any tissue damage, inflammation, or lesions due to toxicity caused by the PLA-PEG-PPG-PEG nanoparticles or their degradation products. No apparent histopathological abnormalities or lesions were observed in the brain, heart, liver, spleen, lung and kidney of the nanoparticle-treated animal, as shown in FIG. 10.

### Example 7: Efficacy of the PLA-PEG-PPG-PEG Nanoparticles as Nanocarrier Systems In-Vivo

Ehrlich Ascites Tumor (EAT) model transgenic mice of strain BALB/c type were used for evaluating the efficacy of the nanoparticles as nanocarrier systems. Animals having body weight of 20 g were taken up for the study (FIG. 12A).

Anticancer peptide drug, NuBCP-9, was loaded into the PLA-PEG-PPG-PEG polymeric nanoparticles. The mice were given an intraperitoneal formulation of the polymeric nanoparticles as prepared in Example 2 comprising the anticancer peptide, NuBCP-9, at a dose of 200-1000 μg of peptide encapsulated in PLA-PEG-PPG-PEG. The total weight of the anticancer peptide given to the animals was 300 μg to 600 μg/mice. The dosing frequency of the formulation was biweekly for a period of 21 days and the animals were kept under observation for a period of 60 days.

Tumor growth suppression was observed in the mice after administration of the nanoparticles loaded with NuBCP-9 for a period of 60 days (FIG. 11). It was found that the mice treated with the NuBCP-9-loaded nanoparticles were completely cured of tumor (FIG. 12b) compared to the control group (FIG. 12c). The control group received plain nanoparticles without any therapeutic agent.

### Evaluation of Insulin Loaded PLA-PEG-PPG-PEG Nanoparticles as Parenteral Depot in Diabetic Rabbits

### Encapsulation of Insulin in PLA-PEG-PPG-PEG Nanoparticles

Insulin encapsulated PLA-PEG-PPG-PEG nanoparticles were prepared by the double emulsion solvent evaporation method. For nanoparticle preparation, 1 g of PLA-PEG-PPG-PEG copolymer was dissolved in acetonitrile. Insulin (500 I.U.) was added to the solution with brief sonication of 15 s to produce a primary emulsion. The resultant primary emulsion was added drop-wise to 30 ml aqueous phase and stirred magnetically at room temperature for 6-8 hours in order to allow solvent evaporation and nanoparticle stabilization. The nanoparticles were collected by centrifugation at 21,000 rpm for 10 min and washed thrice using distilled water. The insulin loaded-PLA-PEG-PPG-PEG nanoparticles were lyophilized and stored at 4° C. until further use.

### In-Vivo Studies

Diabetic rabbits were administered a single dose of 50 I.U./kg body weight insulin loaded PLA-PEG-PPG-PEG nanoparticles, subcutaneously, and monitored for 10 days.

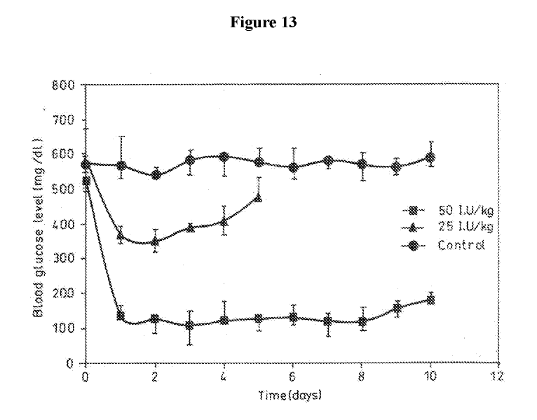

In animals given an insulin dose of 50 I.U./kg body weight, the blood glucose level was maintained between 120-150 mg/dl up to 8 days after which a gradual increase in blood glucose level was observed. The drug loaded polymeric nanoparticles form a depot at the site of injection and release the entrapped insulin in a sustained manner due to slow degradation and diffusion. The glucose level did not revert to original diabetic levels (500 mg/dl) even after 8 days, indicating the capability of polymeric nanoparticles to hold and release bioactive insulin in a sustained manner for more than a one week time period (FIG. 13).

### Evaluation of MUC1 Loaded PLA-PEG-PPG-PEG Nanoparticles:

In-Vitro Release of Encapsulated MUC1 by the Polymeric Nanoparticle PLA-PEG-PPG-PEG

A mixture containing 10 ml phosphate buffer saline and 10 mg PLA-PEG-PPG-PEG nanoparticles encapsulating rhodamine B-conjugated to a MUC1 cytoplasmic domain peptide linked to polyarginine protein transduction domain (Ac-RRRRRRRRRCQCRRKN-NH2) was stirred at 200 rpm at 37° C. Supernatant samples of the mixture were collected by centrifugation at 25,000 rpm at different time intervals for a period of 6 days. The nanoparticles were re-suspended in fresh buffer after each centrifugation. 2 ml of the supernatant was subjected to protein estimation using BCA kit (Pierce, USA) to evaluate the amount of drug release spectrophotometrically at 562 nm. The drug release was calculated by means of a standard calibration curve. It was observed that the release of the drug by the PLA-PEG-PPG-PEG polymeric nanoparticles can be controlled up to 60 days. (FIG. 14).

### The XTT (sodium 2,3,-bis(2-methoxy-4-nitro-5-sulfophenyl)-5-[(phenylamino)-carbonyl]-2H-tetrazolium Inner Salt) Assay

Cell viability using XTT (sodium 2,3,-bis(2-methoxy-4-nitro-5-sulfophenyl)-5-[(phenylamino)-carbonyl]-2H-tetrazolium inner salt) assays were carried out in Primary HUVEC cell lines and the MCF-7 cell lines (Table 6).

A total of 1×10⁴ MCF-7 cells were seeded on each well of a 96-well plate and cultured for 24 h. After 24 hours, cells in each plate were treated with polymeric nanoparticles of the present invention containing either 20 or 30 μM of MUC1-cytoplasmic domain peptide linked to a polyarginine sequence (RRRRRRRRRCQCRRKN) or control nanoparticles without any peptide. The cells were incubated with the nanoparticles for different intervals of time ranging from 16 h, 24 h, 48 h, 72 h and 96 h. After incubation, the medium containing PLA-PEG-PPG-PEG nanoparticles loaded with MUC1-cytoplasmic domain peptide was exchanged with fresh medium, and 10 μl of the reconstitute XTT mixture kit reagent were added to each well. After culturing for 4 h, the absorbance of the sample was measured by using a microtiter plate reader (Bio-Rad, CA, U.S.A.) at 450 nm. The proliferation of cells was determined as the percentage of viable cells of the untreated control and analyzed in triplicate. Table 6 shows the effect of MUC1-cytoplasmic domain peptide-loaded PLA-PEG-PPG-PEG nanoparticle on the cell viability of hormone-dependent breast carcinoma cell line, MCF-7.

Those skilled in the art will recognize, or be able to ascertain using no more than routine experimentation, many equivalents to the specific embodiments of the invention described herein. Such equivalents are intended to be encompassed by the following claims.

### Example 8: Synergistic Effect of PTX and L-NuBCP-9 Peptide Co-Delivered by Polymeric Nanoparticles

Paclitaxel and L-NuBCP-9 were encapsulated into PLA-PEG-PPG-PEG tetrablock polymeric nanoparticles to assess the synergistic effect to malignant cells in vitro and in vivo.

### I. Materials and Methods

A. Synthesis and Characterizations of PLA-PEG-PPG-PEG Copolymers:

PLA-PEG-PPG-PEG terablock copolymer was synthesized using 70-kDa PLA (NatureWorks, USA) or 12-kDa PLA (Purac Chemicals, EUROPE) and Poloxamer-F127 (12.5 KDa) and Poloxomer F68 (6 KDa); (Sigma-Aldrich, USA). Tetrablock copolymer were synthesised by DCC-DMAP (Sigma-Aldrich) method.

B. Preparation of L-NuBCP 9 and PTX Drug Loaded Nanoparticles:

L-NuBCP-9 peptide (custom synthesized from Bioconcept, India) loaded PLA-PEG-PPG-PEG nanoparticles was performed using a double emulsion solvent evaporation method as reported in the previous paper by Kumar M, Gupta D, Singh G, Sharma S, Bhat M, Prashant C K, Dinda A K, Kharbanda S, Kufe D, and Singh H. Cancer Research 74(12): 3271-3281, 2014. PTX loaded nanoparticles were produced using an emulsion-solvent evaporation method. Briefly, 50 mg of copolymer in 5 ml acetonitrile (ACN) and 5 mg of PTX (LC Laboratories, Boston, Mass., US) were dissolved in 100 ul ACN and added to 50 mg of PLA-PEG-PPG-PEG solution in 5 ml ACN. The resulting mixture was then added into a 20 ml aqueous phase composed of F127 in distilled water and stirred at room temperature for 6 to 8 hours to facilitate solvent evaporation and nanoparticle stabilization. PTX and NuBCP-9 peptide loaded PLA-PEG-PPG-PEG (50 mg) nanoparticles were prepared by double emulsion process. Paclitaxel was added into the dissolved PLA-PEG-PPG-PEG copolymer followed by immediate addition of peptide with a slight sonication. Then this whole mixture was added into the 20 ml aqueous phase containing poloxomer F127. Rhodamine (RhB) as hydrophilic and coumarine 6 as hydrophobic dye loaded nanoparticles were also prepared by same procedure for cellular uptake studies of PLA-PEG-PPG-PEG nanoparticles.

Nanoparticles were filtered through an Amikon 30-kDa ultrafilter (Millipore, USA) and washed twice with MQ water to remove free drug/dye. The nanoparticles were lyophilized and stored at −20° C. until use. The filtrate was collected and analyzed for free NuBCP-9 peptide by a Micro-BCA Kit (Pierce Chemicals, USA) and measured on EPOCH microplate reader (BioTek, US) at 590 nm. Similarly, free paclitaxel was measured through high performance liquid chromatography HPLC (Perkin Elmer, US) assay method, using C18 column with acetonitrile, Water, Methanol (60:35:5 volume ratio) as mobile phase. Encapsulation efficiency (EE %) of NuBCP-9 peptide/PTX was determined using the following formula:

$$E E \% = \frac{[ Total Drug ( Peptide / PTX ) - Filtrate ] \times 100}{Total ( Initial Peptide / PTX )}$$

Morphology and particle size of the nanoparticles were determined using scanning electron microscopy (SEM, Zeiss EVO 50 Series) and transmission electron microscope (TEM, Philips Model CM12). Zeta potential of the nanoparticles was assessed by nanoparticle tracking analysis (Malvern nanosight, UK).

C. Assessment of Peptide and Paclitaxel Release from Nanoparticles:

In-vitro release kinetics of NuBCP-9 and paclitaxel from nanoparticles were determined by the ultrafiltration method. Briefly, samples of freeze-dried nanoparticles (10 mg) were suspended in PBS and incubated at 37° C. with constant shaking at 150 to 160 rpm. At predetermined time points of up to 60 days, the samples were removed from the incubator and ultrafiltered through 30-kDa Amikon filters (Millipore). The filtrates were collected for analysis and fresh buffer was added to the respective tubes. Peptide concentration in the filtrates was determined by micro-BCA assay kit and PTX was measured through HPLC.

D. In Vitro Cytotoxicity Analysis:

The in vitro cytotoxicity of PLA-PEG-PPG-PEG nanoparticles was assessed on two cancer cell lines. Human ER+MCF-7 and ER-MDA-MB231 breast cancer cells were grown in DMEM containing 10% FBS, 100 units/mL penicillin, and 100 g/mL streptomycin. The cells were maintained at 37° C. and 5% CO2 atmosphere for the duration of experiments. Exponentially growing cancer cells were plated into a 96-well plate at a seeding density of 3000 cells per well and incubated for 24 hrs. Free PTX in DMSO, PTX- and NuBCP-9 loaded (single/dual) in PLA-PEG-PPG-PEG nanoparticles was added separately at final drug concentrations of 0.001, 0.01, 0.1, 1, 5, 10 and 20 μM in the wells. The final level of DMSO in the culture plate wells was <0.1% after dilution with cell culture medium. Tumor cell proliferation inhibition behavior of free drug, drug-loaded single or dual drug PLA-PEG-PPG-PEG nanoparticles were evaluated separately after 72 hrs by XTT based in vitro cell proliferation Assay Kit (Cayman, USA) as per manufacturer instructions. The half maximal inhibitory drug concentration (IC50) was determined by the median effect equation using Graph Pad prism and data are presented as average±SD (n=3).

To assess the uptake of nanoparticles, MCF-7 cells were seeded on coverslips and grown for 24 hours and then incubated with rhodamine B and coumarin 6 loaded nanoparticles, the coverslips were removed, washed with PBS, and fixed with 4% paraformaldehyde. The cells were then stained with 4,6-diamidino-2-phenylindole (DAPI) (Invitrogen, US) and visualized under a confocal laser scanning microscope (CLSM; Olympus, Fluoview FV1000 Microscope).

E. Combination Index (CI) Analysis:

CI analysis based on Chou and Talalay method was performed using compusyn software (version 1.0, combosyn Inc., U.S.) for PTX and NubCP-9 peptide combination, determining synergistic, additive or antagonistic cytotoxic effects against MCF-7 and MDA-MB-231 breast cancer cells.

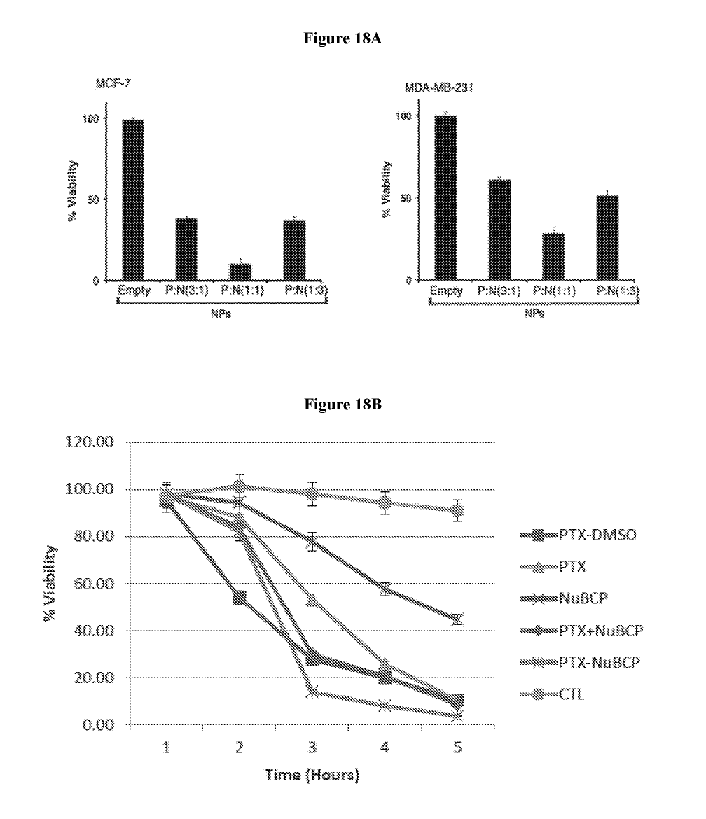

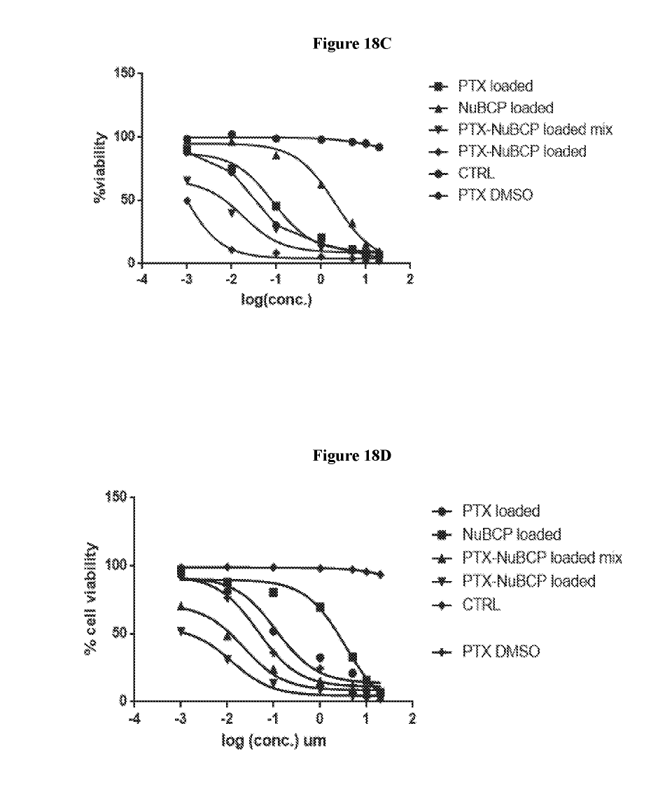

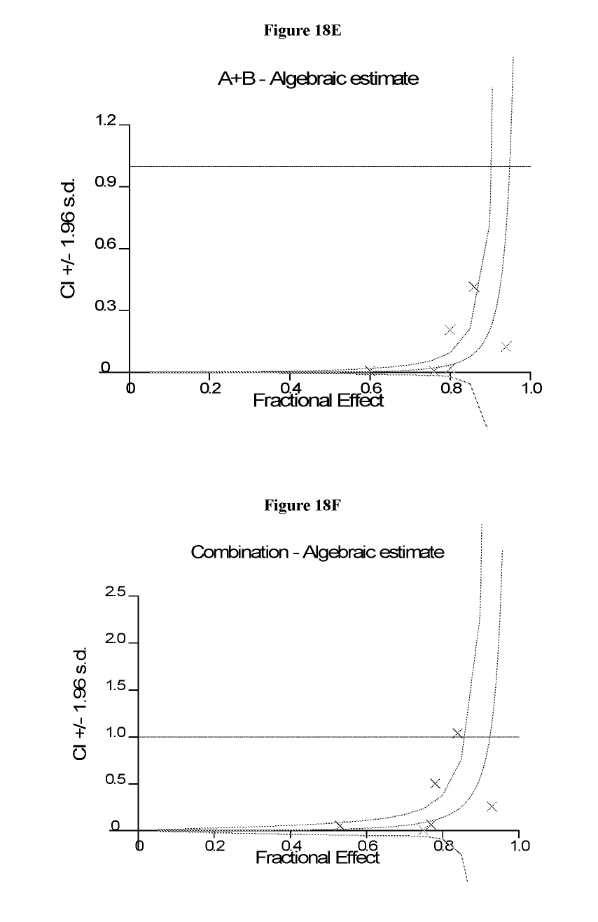

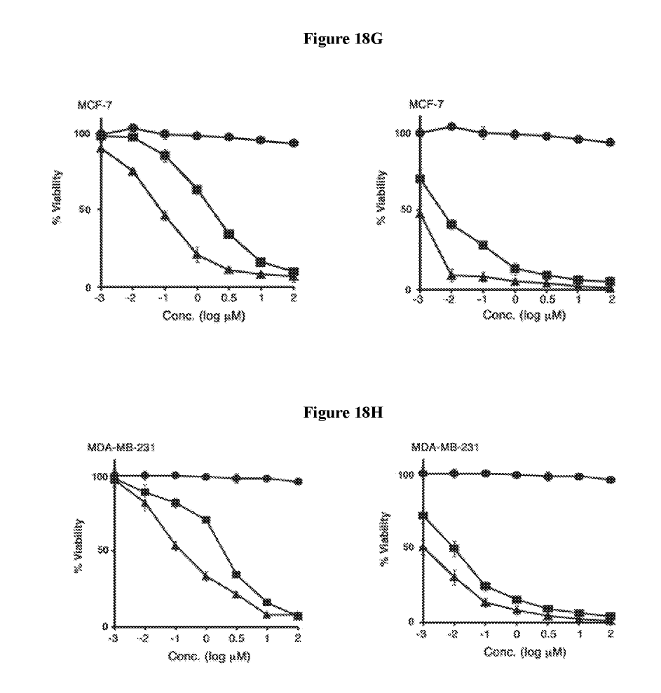

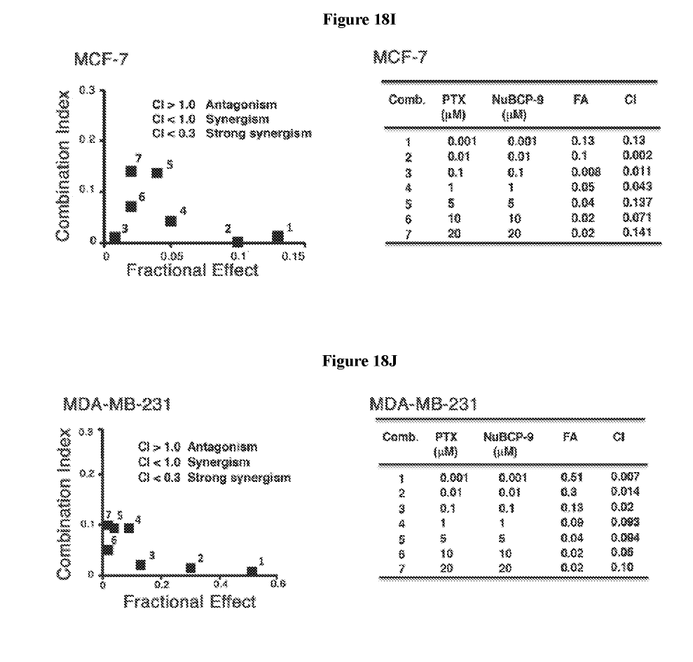

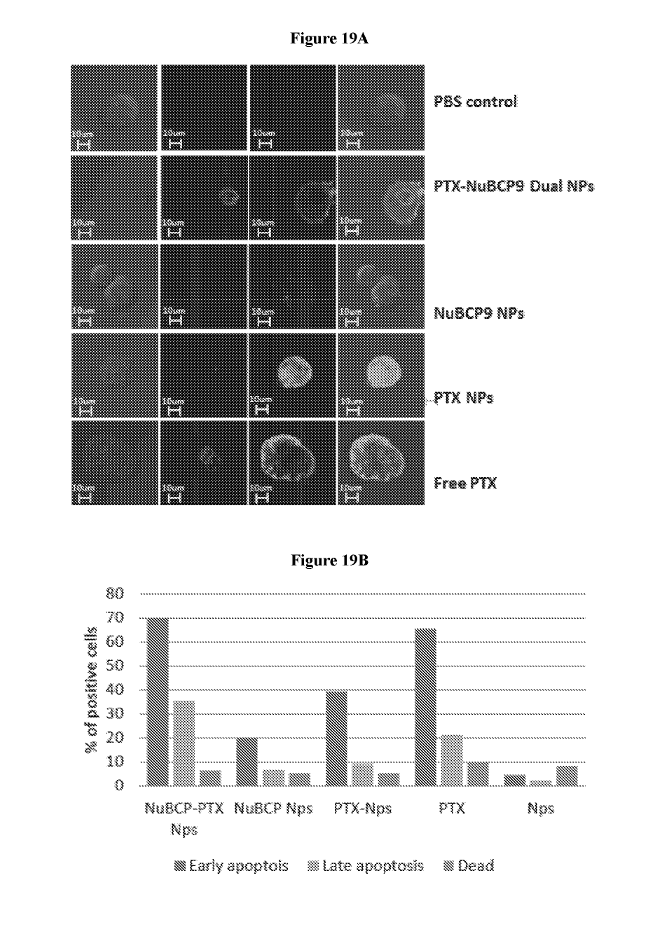

Values of CI>1 represents antagonism, CI=1 additive and CI<1 represent synergism. At constant drug combination ratios, fa (fractional affect) versus CI plots for the drug combination were obtained with GraphPad prism software (Version 5.0, U.S.) (e.g., FIGS. 18E, 18F, 18I, 18J).

F. Assessment of Apoptosis:

Cells were stained using the Annexin V-Alexa Fluor 488/PI Apoptosis Assay Kit (Invitrogen, USA). For qualitative analysis, cells were imaged using the CLSM microsocope. Quantification of apoptosis/necrosis was performed using FACS (Aria LLC).

G. Western Blot Analysis:

Cell lysates were prepared with M-PER reagent (Pierce Chemicals, USA) and analyzed by immunoblotting with anti-Bcl-2, anti-β-tubulin, anti-caspase-3 (Biosepses, China), anti-PARP and anti-β-actin (Santa Cruz Biotechnology, USA). Relative fold change in the band intensity was calculated from the software of chemiliumincsence (Li-Cor blot scanner, USA)

H. Analysis of Antitumor Activity:

Mice Ehrlich tumor cells were injected subcutaneously in the hind limb of syngeneic Balb/c mice (17-22 g). Tumor bearing mice (˜150 mm³) were divided into 9 groups (6 mice/group) and treated weekly or biweekly intraperitoneally (i.p.) with different formulations for 21 days. Tumor volume was determined by vernier caliper and calculated using the formula (A×B²)×0.5, where A and B are the longest and shortest tumor diameters, respectively. From each group, 1 mouse was sacrificed on day 7, 14, and 21 for harvesting of tumor for histopathologal examination. The tumors were fixed in 10% formalin/saline and embedded in paraffin. Five-micrometer sections were stained with hematoxylin and eosin for further immunohistochemistry, TUNNEL and Ki67 assay. Statistical analysis of tumor volumes was performed by one-way ANOVA using Graph pad prism. Survival of the mice was determined by the Kaplan-Meier method using Prism 4.0 software (Graph Pad Software).

I. Data/Statistical Analysis:

All the results are reported as mean±standard deviation and the difference between the control and test groups were tested using students t test. Sample size of at least three was used for the analysis. Results were considered statistically significant between the control and test treatment at the level of P<0.05.

### II. Results

A. Preparation and Characterization of NuBCP-9-Loaded Polymeric Nanoparticles:

PLA-PEG-PPG-PEG block copolymers were prepared using PLA of 12 KDa or 72 KDa and PEG-PPG-PEG block of 6 KDa or 12.5 KDa using DCC DMAP as described previously. The PLA^{12K} PEG-PPG-PEG and PLA^{72K} PEG-PPG-PEG was found to be 15.6 KDa and 83 KDa Synthesis of bock copolymers was confirmed by 1HNMR as previously mentioned.

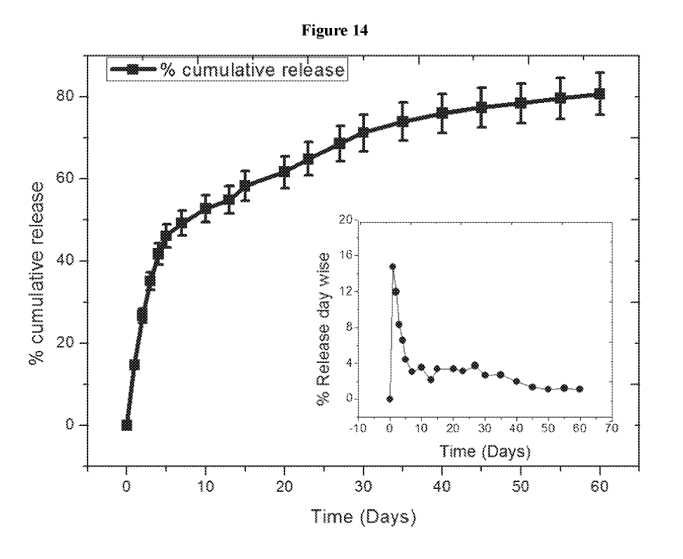

The morphology and size of PLA-PEG-PPG-PEG tetrablock copolymer was analysed through SEM and TEM. SEM showed spherical morphology of PLA-PEG-PPG-PEG nanoparticles while TEM showed the multi-layered structure where PLA exists as hydrophobic core and the PEG as hydrophilic shell with a hydrophobic PPG sandwich between the two layers. Particle sizes ranged from 45-90 nm in diameter (FIGS. 15A and 15B).

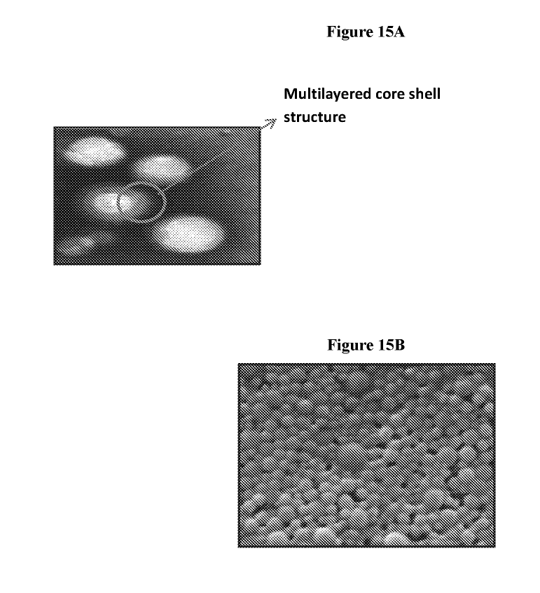

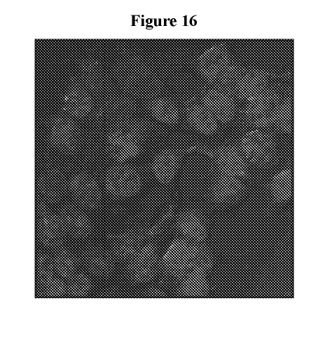

Incubation of rhodamine B (model for hydrophilic drug) and coumarin 6 (model for hydrophobic drug) dye loaded PLA^{72K}-PEG-PPG-PEG^{12.5K} nanoparticles with MCF-7 breast cancer cells were exhibited the uptake of nanoparticles after 3 hrs by fluorescent confocal laser scanning microscopy (FIG. 16). The results showed the intracellular fluorescence of the rhodamine B and coumarin 6 loaded nanoparticles throughout the cytosol. Based on these observations uptake of PLA-based NPs is through endocytosis and is associated with surface charge reversal (anionic to cationic) in the acidic pH of the endo-lysosomes. This charge reversal facilitates interaction of the NPs with vesicular membranes, leading to transient and localized membrane destabilization, and thereby escape of the NPs into the cytosol (Kumar M, et al. (2014) Novel polymeric nanoparticles for intracellular delivery of peptide cargos: antitumor efficacy of the BCL-2 conversion peptide NuBCP-9. Cancer Res 74(12):1-11; Hasegawa M, et al. (2015) Intracellular targeting of the oncogenic MUC1-C protein with a novel GO-203 nanoparticle formulation. Clin Cancer Res 21(10):2338-2347).

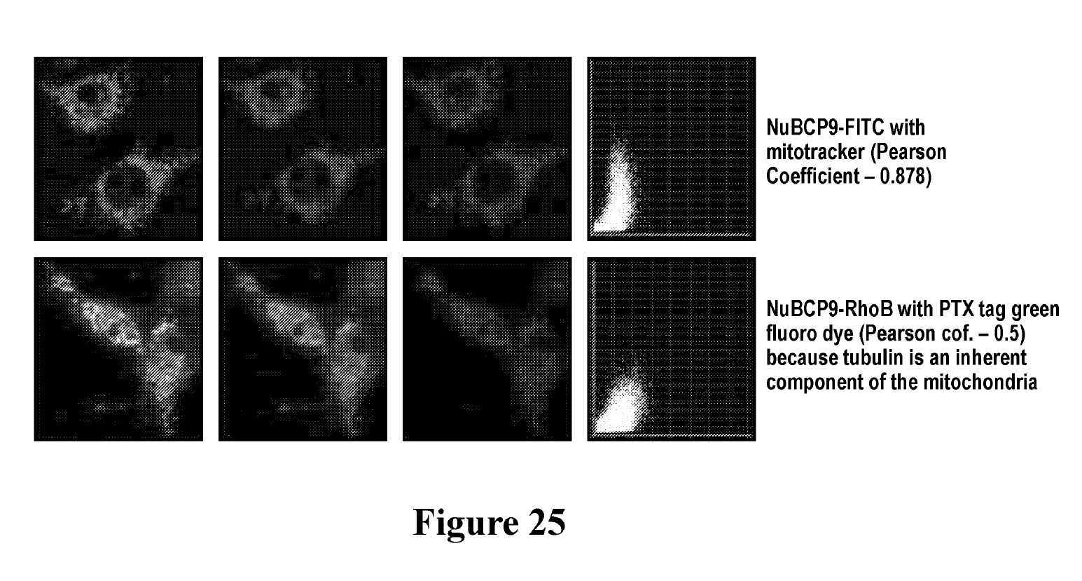

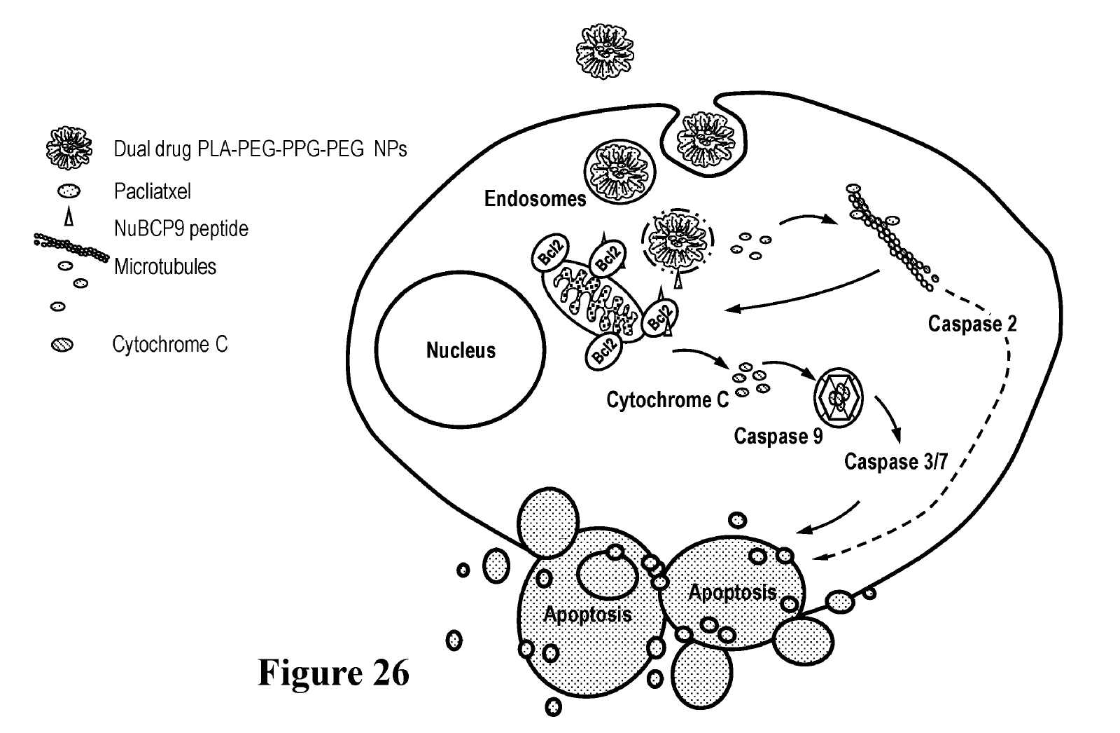

NuBCP-9 targets BCL-2 to convert it from a cell protector to cell killer (Kolluri S K, et al. (2008) A short Nur77-derived peptide converts Bcl-2 from a protector to a killer. Cancer Cell 14(4):285-298). Accordingly, the intracellular localization of NuBCP-9 when treating MCF-7 cells with FITC-NuBCP-9/NPs was investigated. FITC-NuBCP-9 localized to the cytoplasm and mitochondria as evidenced by staining with Mitotracker (FIG. 25). Photoaffinity crosslinking studies have demonstrated localization of PTX binding to tubulin in microtubules (Rao S, et al. (1995) Characterization of the taxol binding site on the microtubule. 2-(m-Azidobenzoyl) taxol photolabels a peptide (amino acids 217-231) of beta-tubulin. J. Biol. Chem. 270(35):20235-20238; Rao S, et al. (1999) Characterization of the Taxol binding site on the microtubule. Identification of Arg(282) in beta-tubulin as the site of photoincorporation of a 7-benzophenone analogue of Taxol. J. Biol. Chem. 274(53):37990-37994) and in mitochondria (Carre M, et al. (2002) Tubulin is an inherent component of mitochondrial membranes that interacts with the voltage-dependent anion channel. J. Biol. Chem. 277(37):33664-33669). In agreement with those and the above studies, confocal analysis of MCF-7 cells treated with FITC-PTX/NPs and RhoB-NuBCP-9/NPs demonstrated the colocalization of PTX and NuBCP-9 in the cytosol and mitochondria (FIG. 25).

B. Drug Loading Efficiency and In Vitro Release Studies

The percentage encapsulation of NuBCP-9 and PTX with different molecular weight PLA-PEG-PPG-PEG nanoparticles are listed in Table 7. PLA-PEG-PPG-PEG tetra block copolymer is highly hydrophobic due to its high PLA content (84%), which resulted in a low encapsulation of hydrophilic peptide NuBCP-9 with 64.5% as compared to Paclitaxel i.e. 87%.

Different formulations of PTX-NuBCP-9 peptide combination in PLA-PEG-PPG-PEG nanoparticles were prepared with an aim to achieve the maximum cell proliferation inhibition at minimum concentrations of PTX and NuBCP-9 peptide. Further, these formulations were observed for their size and zeta potential as given in Table 7. The encapsulation efficiency of PTX was determined to be >90% in all the formulations whereas in case of NuBCP-9 peptide the loading increased with the increase in peptide amount. Among all the formulations, maximum loading was observed in 1:4 ratio of PTX and NuBCP-9 peptide, respectively, subsequent increase in ratio leads to micro particle formation.

The zeta potential of nanoparticles was also observed to be more negative with increase of peptide encapsulation (Table 7) The precise reason for this decrease in z potential is not clear; however, it is plausible that because of the interaction of the positively charged adsorbed peptide with the negatively charged PLA, the peptide carboxyl groups, which have a negative charge. Size distribution of PTX and NuBCP-9 loaded PLA-PEG-PPG-PEG NPs (single/dual) ranges from 100-170 nm, which was observed to be almost similar in all the formulations.

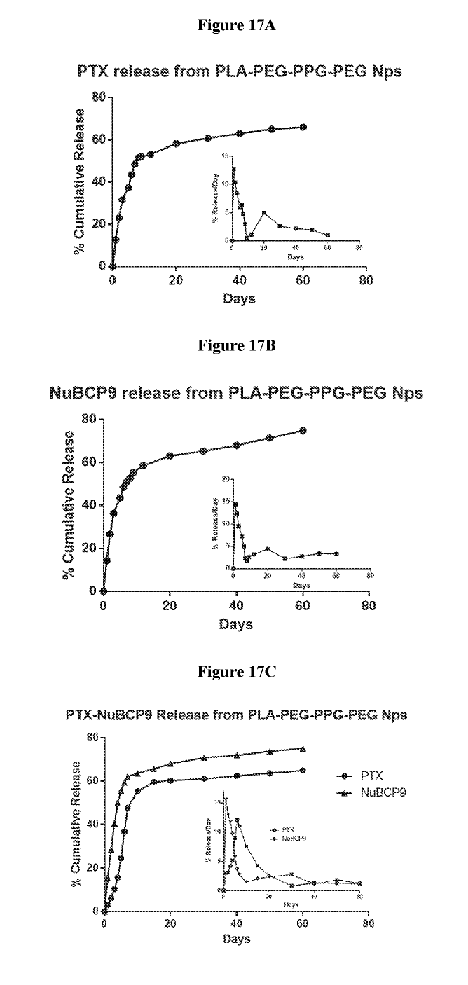

In vitro release profiles for PTX and NuBCP-9 from PLA^{72K1/12K}-PEG-PPG-PEG^{12.5k} nanoparticles is shown in FIGS. 17A and 17B. The co-release of PTX and NuBCP-9 peptide from PLA^{72K}-PEG-PPG-PEG^{12.5K} and PLA^{12K}-PEG-PPG-PEG^{6K} at physiological pH showed a slow and sustained cumulative release of 30% and 40% of drugs respectively within 7 days, whereas it was 47% for PTX and 58% for peptide when loaded as single drugs in nanoparticles (FIG. 17C). However, a complete in-vitro release profile for PTX and NuBCP-9 (single/dual) from low molecular weight PLA^{12K}-PEG-PPG-PEG^{6K} was sustained for only 7 and 10 days, respectively, as compared with 60 days with high molecular weight PLA^{72K}-PEGPPG-PEG^{12.5K} probably due to faster degradation and bio-solubilization of low molecular weight PLA.

These findings demonstrate that (i) encapsulation of both PTX and NuBCP-9 is achievable in the same NPs and (ii) release of PTX and NuBCP-9 is sustained from PTX-NuBCP-9/NPs.

Based on these results, the NuBCP-9 and PTX-encapsulated (single and dual) PLA^{72K}-PEG-PPG-PEG^{12.5K} nanoparticles were taken further for controlled and sustained delivery of drugs for longer period of time as compared to the low molecular weight PLA tetra block nanoparticles further studied for biologic activity in vitro and in vivo studies.

C. In Vitro Cytotoxicity and Combinational Analysis

To verify the synergistic effect of the co-delivery system, different formulations were studied in a dose dependent manner for in vitro cell viability effects against free drug and single/dual drug-loaded nanoparticles on MCF7 and MDA-MB231 cells. As shown in FIG. 18A, it was observed that 1:1 formulation of PTX-NuBCP-9 combination loaded nanoparticles showed highest cell proliferation inhibition on both MCF7 and MDA-MB231 breast cancer cells as compared to other various drug formulations. Therefore, 1:1 formulation of PTX-L-NuBCP-9 loaded nanoparticles was being used for further in vitro and in vivo studies.

A time dependent study was performed up to 96 hours to compare the efficacy of free versus drug loaded (single/dual) NPs at 1 uM. In FIG. 18B, 1:1 combination PTX-NuBCP-9 peptide loaded NPs showed >80% cell inhibition at 48 hours. PTX loaded and L-NuBCP-9 loaded mix together showed ˜70% inhibition which is comparable with free PTX. Although, single loaded PTX and NuBCP-9 showed only 40% and 20% cell proliferation inhibition respectively. Hence, the synergistic effect of 1:1 PTX-NuBCP-9 peptide combination loaded nanoparticles was confirmed by cell proliferation inhibition at 48 h. The cell viability of plain PLA-PEG-PPG-PEG nanoparticles was also tested at different concentrations up to 115 μM which was above 85%, indicating that non toxicity and biocompatibility of PLA-PEG-PPG-PEG nanoparticles (FIGS. 18C and 18D).

Single drug PTX and NuBCP-9 peptide loaded nanoparticles were mixed in 1:1 ratio to compare with dual PTX-NuBCP-9 peptide loaded nanoparticles and evaluated for cell proliferation inhibition studies. Mixed nanoparticles showed only 70% inhibition as compared to 90% inhibition by dual loaded PTX-NuBCP-9 peptide loaded NPs at 48^{th} hr. When single drug loaded nanoparticles were mixed in same ratio is almost ineffective at 1 uM whereas when both the drugs loaded together in same nanoparticles, showed maximum synergistic effect which was far better than the single PTX or NuBCP-9 loaded NPs. Therefore, the synergistic effect of dual loaded nanoformulation was confirmed.

In view of the foregoing, it can be concluded optimized co-delivery of PTX-NuBCP-9 NPs into the cells is very important for enhanced antitumor efficacy in vitro.

Combination Index of different nanoformulations were analysed at wide range of concentrations on MCF-7 and MDA-MB cells. Combination index (CI) values lower than, equal to, or higher than 1 indicate synergism, additivity, or antagonism, respectively. It was observed that 1:1 nanoformulation of PTX-NuBCP-9 peptide loaded nanoparticles has best fit levels of high synergism as compared with free or single drug loaded nanoparticles (FIGS. 18E and 18F). To further substantiate these findings, MCF-7 cells were treated with different concentrations of PTX/NPs, NuBCP-9/NPs or PTX-NuBCP-9/NPs. CI analysis based on the Chou and Talalay method was performed using Compusyn software. The results demonstrate that all of the different combinations are synergistic with CI values <0.2 (FIG. 18I). Similar results were obtained with MDA-MB-231 cells (FIG. 18J) indicating that PTX-NuBCP-9/NPs are synergistic in inhibiting growth and survival of breast carcinoma cells.

D. Effect of NuBCP-9-PTX Combination Loaded Nanoparticles on Apoptosis of Breast Cancer Cells:

To assess the effects of PTX-NuBCP-9 nanoparticles on the apoptotic effect with single or dual loaded PLA-PEG-PPG-PEG nanoparticles, MCF-7 cells were treated with nanoparticles and monitored for externalization of phosphatidylserine at the cell membrane. Confocal images of MCF-7 cells stained with Annexin V-Alexa flour 488/PI demonstrated that the treatment with combination PTX-NuBCP-9 and single drug loaded nanoparticles resulted in higher apoptosis than single loaded nanoparticles at 48 h is associated with the induction of an apoptotic response. By contrast, treatment with empty nanoparticles had no apparent effect.

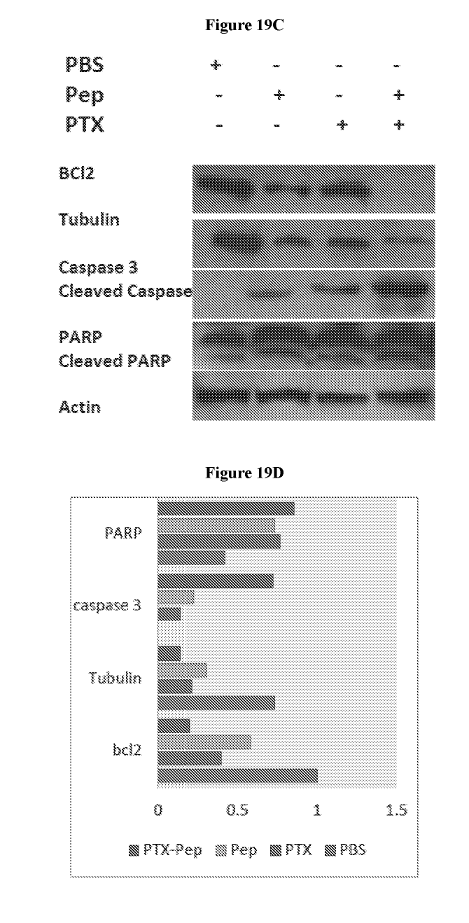

Quantitation of Annexin V and PI staining by FACS (Aria BD falcon) further confirmed the combination of PTX-NuBCP-9 PLA^{72K}-PEG-PPG-PEG^{12.5K} nanoparticle is more effective than single loaded nanoparticles in inducing apoptosis of MCF-7 cells at 24 hours (FIGS. 19A/19B).

The levels of BCL-2, Tubulin, cleaved fragment of caspase 3 and cleaved fragment of PARP proteins in the breast cancer cell lines were examined through Western blot analysis. (FIG. 19C) The combination of PTX-NuBCP-9 nanoformulation has reduced BCL-2 and Tubulin expression levels and increases cleaved fragment of caspase 3 and cleaved fragment of PARP expression more than either single drug loaded nanoparticles alone (FIG. 19D). These findings support the premise that PTX-NuBCP-9/NPs are more active in inducing apoptosis of MCF-7 cells than PTX/NPs or NuBCP-9/NPs.

E. Evaluation of Synergistic Antitumor Efficacy In Vivo:

The in vivo antitumor efficacy and systemic toxicity of the dual-drug and single drug loaded nanoparticles were evaluated on EAT tumor model Balb/c mice. Accurate administration of the viscous suspensions of drug loded nanoparticles was problematic in the narrow tail vein and therefore, used the i.p. route of administration was used, which allows nanoparticles to enter the systemic circulation through mesenteric vessels and the portal vein. Mice were treated with different drug formulations, loaded individually and in combination, given at biweekly and weekly schedules for up to 21 days and exhibited significant effect on tumor growth as compared with that obtained in the saline control.

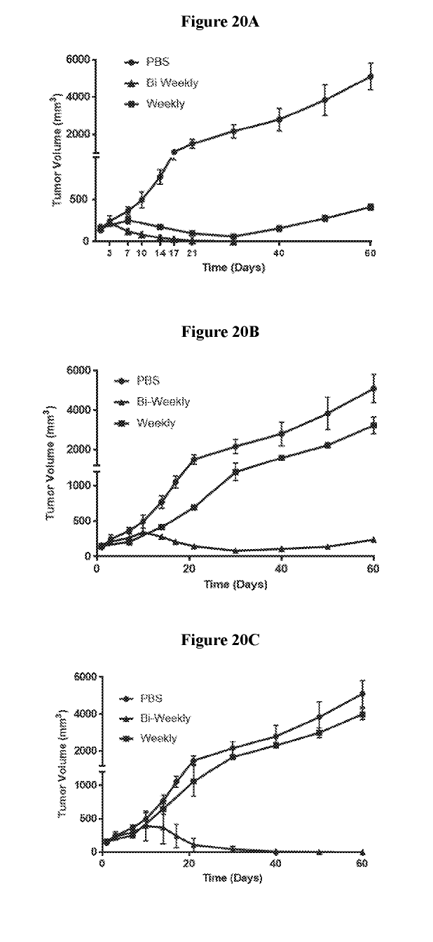

The previous studies were performed using NuBCP-9 peptide alone and NuBCP-9 loaded PLA^{72K}-PEG-PPG-PEG12.5k NPs by giving 20 mg/kg biweekly i.p injections, showed almost 90% regression in the tumour volume. In comparison to that combination of NuBCP-9-PTX showed the better efficacy with complete inhibition of tumor growth and no obvious tumour recrudescence during the whole treatment. It was also observed that in all the three sets (dual-, PTX-, NuBCP-9 peptide loaded NPs) biweekly i.p administration showed more efficacy than weekly administration (FIGS. 20A, 20B, and 20C) Significantly, there was no weight loss or other overt toxicities observed in any of the PTX/NuBCP-9 nanoparticles (single/dual) treated mice.

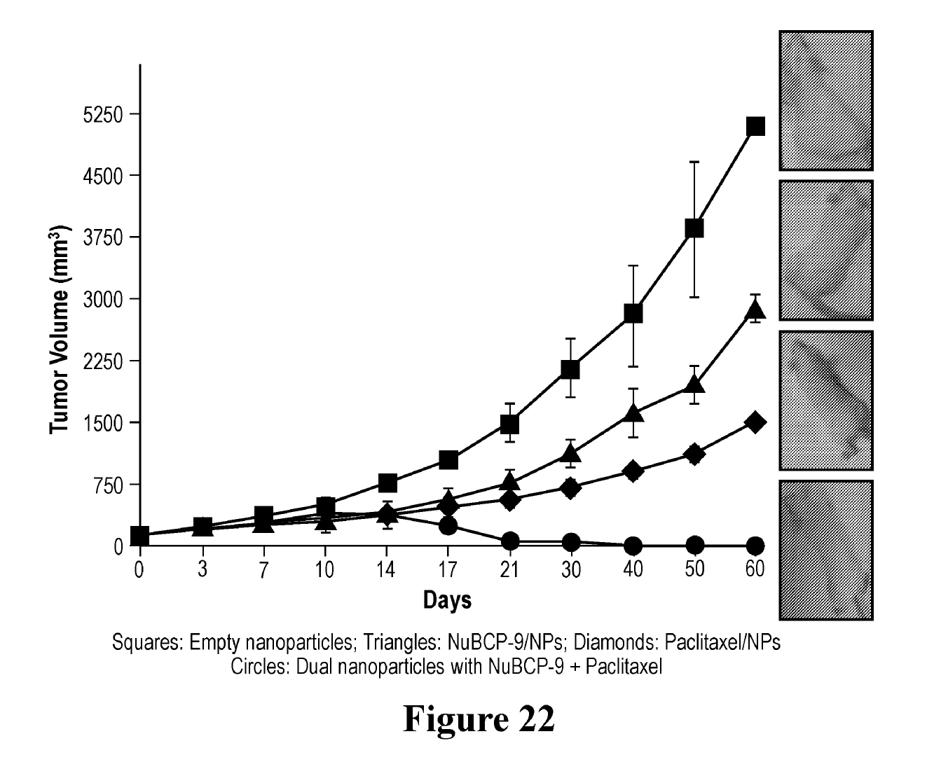

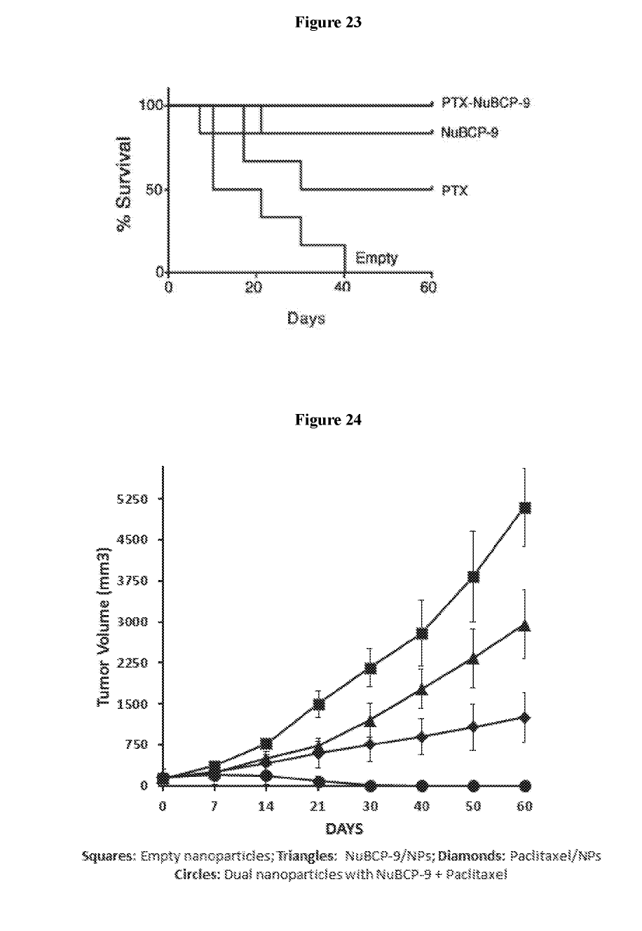

Ehrlich tumor-bearing mice were treated i.p. twice a week for 3 weeks. As compared with mice treated with empty NPs, treatment with 10 mg/kg PTX/NPs was associated with partial regression of the tumors (FIG. 22). Moreover and importantly, treatment with 10 mg/kg PTX-NuBCP-9/NPs was associated with complete and prolonged tumor regressions (FIG. 22). Analysis of survival as determined by Kaplan-Meier plots further demonstrated that mice treated with PTX-NuBCP-9/NPs survived significantly longer than those treated with empty NPs, PTX/NPs or NuBCP-9/NPs (FIG. 23). With the high antitumor efficacy and the low drug-related toxicity, the dual-drug loaded system is promising in cancer therapy. The principle of drug combination is to achieve efficient antitumor effect at lower drug doses and obtain the maximal therapeutic effect while decreasing negative side effect.

F. Histological and Immunohistochemical Analyses:

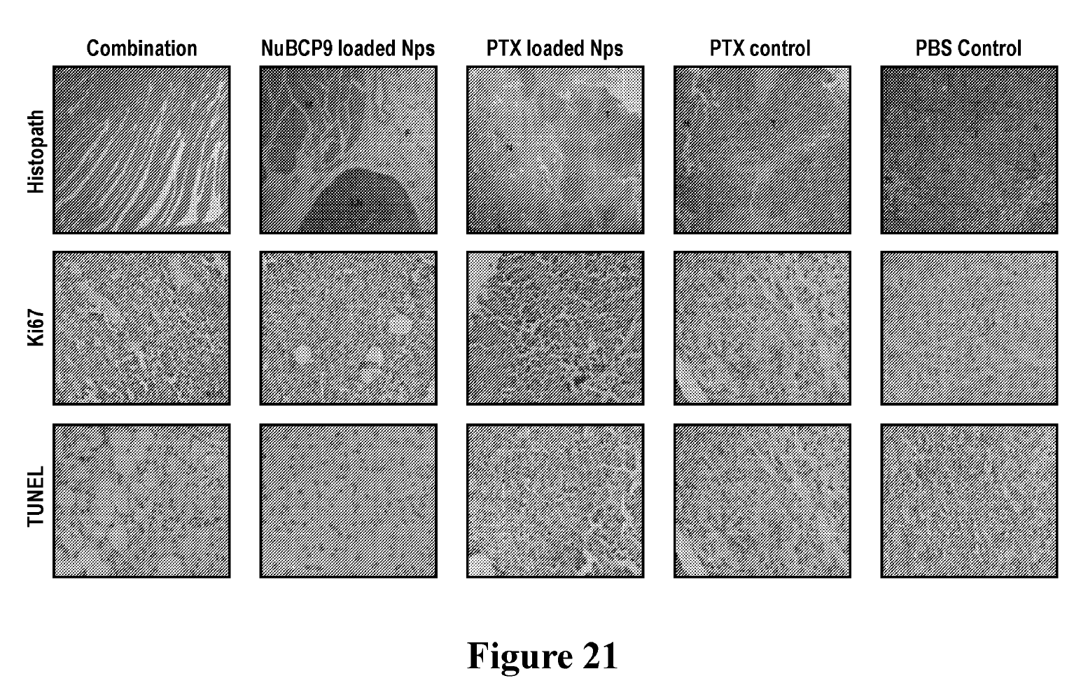

To further investigate the antitumor activity of Co-NPs, tumor-bearing Balb/C mice were sacrificed after the treatment (day 21) and the tumors were dissected and stained with H&E and TUNEL for pathology analysis. The data of PBS, NuBCP-NPs, PTX-NPs and Co-NPs treated groups were shown in FIG. 21.

For H&E staining, the normal tumor cells had large nuclei with spherical or spindle shape and more chromatin. Whereas the necrotic cells did not have clear cell morphology, and the chromatin became darker and pyknotic or absent outside the cellular. As shown in FIG. 7, the tumor cells with normal shape and more chromatin were observed in the PBS group, revealing a vigorous tumor growth. However, the extensive tissue necrosis was observed in single loaded PTX or NuBCP 9 NPs treated groups. However, the Co-NPs treated group had not shown the normal muscle tissue revealing the complete regression of tumor as compared with the groups treated with NuBCP-9-NPs and PTX-NPs, indicating that most tumor cells were necrotic in the Co-NPs treated group.

The TUNEL assay could detect DNA fragmentation in the nuclei of tumor cells. Little apoptosis was detected in the PBS treated tumor tissues. While in the NuBCP-9-NPs, PTX-NPs and Co-NPs treated groups, obvious cell apoptosis areas were observed. The treatment of Co-NPs obviously increased apoptosis level compared with the signal drug-loaded nanoparticles, which was consistent with the H&E analysis.

### III. Discussion

Paclitaxel has been a major chemotherapeutic agent for breast cancer and a variety of solid tumors. The major clinical limitations of paclitaxel are neurotoxicity and cellular resistance after prolonged treatment. NuBCP-9 peptide is a novel epigenetic agent with a dual effect of BCL-2 mediated apoptosis Cancer Cell 2008; 14:285-298. Example 8 demonstrates that paclitaxel and NuBCP-9 have a profound synergistic inhibitory effect on the growth of two different breast cancer cell lines, MCF-7 and MDA-MB-231 when delivered by nanoparticles. The IC₅₀ of NuBCP-9 and PTX decrease dramatically when the two agents are used in combination. The results suggest that it is possible to significantly reduce side effects of PTX while maintaining or enhancing clinical efficacy by combining the two drugs.

In order to characterize the dual drug-loaded PLA-PEG-PPG NPs, the loading degree of Paclitaxel, NuBCP-9, and PTX-NuBCP-9 nanoparticles together in different molecular weight PLA72 KDa/12 KDa with PEG-PPG-PEG12.5k/6K NPs was determined and their in vitro release properties were investigated. The average loading degrees of PTX and NuBCP-9 with different PLA-PEG-PPG-PEG NPs are listed in Table 7. As presented in Table 7, regardless of the loaded drug, the loading degrees for high molecular weight PLA-PEG-PPG-PEG NPs were always higher than their corresponding low molecular weight PLA-PEG-PPG-PEG NPs. Furthermore, the loading degrees for the individual drug molecules were lower in the dual drug-loading (PLA12K-PEG-PPG-PEG-PTX-PEP and PLA72K-PEG-PPG-PTX-PEP) than in the single drug-loading (PLA10KPEG-PPG-PEG-PTX/PLA10KPEG-PPG-PEG-PEP and PLA72K-PEG-PPG-PTX/PLA72K-PEG-PPG-PEP). Therefore, the differences in the loading degree could be attributed to their different intensity of electrostatic attraction and hydrophobic forces between payloads.

The obtained higher loading degree of PTX in the dual drug-loading procedure might be ascribed to the later loading of NuBCP-9 peptide, which could lead to slight release of the NuBCP-9 from the NPs. Following the theory of like dissolves like, due to the hydrophobic nature of PTX captures the hydrophobic core of PLA more than the peptide and also PTX causes some steric hindrance which resulted in a lower loading degree compared with the single drug-loading. On the other hand, the overall loading degree of dual drug-loaded PLA-PEG-PPG-PEG NPs was higher than that of single drug-loaded ones. This could probably be ascribed to the incompletely filled pores of PLA-PEG-PPG-PEG NPs after loading with PTX. In addition, Peptide could also adsorb onto the external surface of the PTX-loaded PLA-PEG-PPG-PEG NPs through hydrophobic interactions between the hydrophobic parts of peptide and the surface of the particles even if all the pores were filled or blocked with PTX. The electrostatic attraction is also a plausible explanation. The isoelectric point of NuBCP-9 is around pH 7.2, which means that the peptide in water should be positively charged during the loading process. Since the PTX-loaded PLA-PEG-PPG-PEG (−3.21±1.5 mV) was negatively charged, peptide could be adsorbed onto the PLA-PEG-PPG-PEG particle surface also through electrostatic attractions, even though PTX was loaded initially and prevented the peptide diffusion into the pores.

The release profiles of PTX and NuBCP-9 peptide from high and low molecular weight PLA-PEG-PPG-PEGS NPs at pH-7.4 are shown in FIG. 3. Peptide and PTX (single or dual) can slowly be released up to 60 days from high molecular weight PLA-PEG-PPG-PEG nanoparticles particles whereas low molecular weight PLA-PEG-PPG-PEG particles could not show the stability due to precipitation of drugs or faster degradation of copolymers. It was suggested that the synergistic effect might result from then combination of individual antitumor mechanism for each drug. As mentioned, NuBCP-9 binds the BCL-2 cascade, thereby converting the protein from pro-apoptotic to anti-apoptotic whereas PTX can inhibit microtubules disassembly which disrupts normal dynamic reorganization of the microtubule network required for mitosis and cell proliferation, and in turn causing cell apoptosis. It was also reported; PTX directly binds to BCL-2 and functionally mimics the activity of Nur 77. As reported, multiple drugs have same cellular pathways could function synergistically for higher therapeutic efficacy and higher target selectivity.

Treating MCF-7 cells with a mix of NuBCP-9 and PTX Nps together works similarly as that of PTX, but when both the therapeutic agents were co-delivered in same vehicle to act concomitantly, best synergistic effects were achieved (FIGS. 18G and 18H, right panels). According to the in vitro studies when using other drug ratios, the synergistic effects could not display efficiently, and balanced dosage of the two drugs together gave the highest tumor efficacy.

It is shown that the IC₅₀ of PTX-NuBCP-9 Nps decreases dramatically in MCF-7 cells and MDA-MB231 triple negative cells with about a 40 and 4-fold differences as compared with normal paclitaxel (Table 8). Therefore co-treatment suggests the ability to potentiate the cytotoxicity of paclitaxel, which offers the potential to broaden the clinical efficacy.

To explore the possible mechanism, in vitro studies demonstrated the synergistic effect of co-delivery of two drugs in same vehicle. The expression of proapoptotic BCL-2 and tubulin remarkably decreases with combined NuBCP-9-PTX loaded Nps as compared to single drug only. These biochemical data provided the foundation of the mechanisms for the synergistic effects of the two agents on apoptosis and cell cycle arrest.

To further explore the possible apoptosis pathways, the expressions of some key apoptosis-associated proteins, including Caspase-3, and PARP were assayed. In this study, the level of Caspase-3, and PARP proteins was remarkably elevated in dual drug loaded NPs group compared to single loaded groups. The cleaved-PARP is critically involved in the intrinsic apoptosis pathway and considered to be a marker of apoptosis.

Previous studies of the antitumor effects of NuBCP-9 nanoparticles administered via i.p showed prolonged tumor regression. In vivo, the tumor volume of the mice injected with dual drug loaded NPs was almost clear than that of the mice treated with single loaded NPs wherein only Nps/saline did not affect the tumor volume in mice (FIGS. 20A-C). These results indicated that dual drug loaded NPs showed more significant antitumor activity. Further, it can be inferred that dual/single loaded NPs deliver the drugs effectively into tumor cells with a sustained and control release for up to 60 days. Of further importance, administration of single/dual Nps was well tolerated with no evidence of weight loss or overt toxicities. These results may advocate the feasibility of reducing the dose and intensifying the tumor-tissue reduction through dual loaded nanoformulation.

### IV. CONCLUSION

In summary, a polylactic acid (PLA) tetrablock with PEG-PPG-PEG copolymer for the co-delivery of NuBCP-9 (anticancer peptide) and PTX has been developed. The robust construct stability, efficiently delivering capacity, good biocompatibility and favourable size distribution of high molecular weight PLA-PEG-PPG-PEG revealed its great potential for delivering antitumor drugs via intraperitoneal injection in cancer treatment. Co-NPs had synergistic effect in suppression of MCF-7 and in triple negative MDA-MB231 breast cancer cell growth. Co-NPs exhibited high tumor accumulation, superior antitumor efficiency and much lower toxicity in vivo. The present studies indicate that the co-delivery system provides a promising platform as a combination therapy in the treatment of breast cancer, and possibly other type of cancer as well.

### Example 9: PTX-NuBCP-9/NPs are Active Against MCF-7 Cells Resistant to PTX and Nab-Paclitaxel

Paclitaxel (PTX) is a widely used microtubule inhibitor for the treatment of breast and other cancers. PTX is also administered in an albumin-bound nanoparticle formulation (nab-paclitaxel; Abraxane). However, the effectiveness of PTX is limited by resistance mechanisms mediated by upregulation of drug efflux pumps, such as P-glycoprotein (P-gp), and the anti-apoptotic BCL-2 proteins. The biodegradable tetrablock polymeric nanoparticles for intracellular PTX delivery (PTX/NPs) described herein are highly effective in inhibiting PTX efflux. Specifically, the PTX/NPs are active against P-gp-expressing breast cancer cells resistant to PTX and nab-paclitaxel. These nanoparticles have been used to systemically deliver the NuBCP-9 peptide (NuBCP-9/NPs), which converts the anti-apoptotic BCL-2 protein from a cell protector to cell killer. Treatment of breast cancer cells with NPs containing both PTX and NuBCP-9 (PTX-NuBCP-9/NPs) is markedly synergistic against breast cancer cells in vitro, as evidenced by a 40-fold decrease in the PTX IC₅₀ and an enhanced apoptotic response. Treatment of the syngeneic Ehrlich breast tumor model in mice with PTX-NuBCP-9/NPs was also significantly more effective than that obtained with either PTX/NPs and/or NuBCP-9/NPs (see Example 8). These results demonstrate that PTX/NPs are active in the setting of nab-paclitaxel resistance and that the activity of PTX is synergistically increased when codelivered with NuBCP-9 in PTX-NuBCP-9/NPs. These findings also support the notion that this platform could be broadly applicable for enhancing the activity of other cytotoxic agents that are P-gp substrates and/or inhibited by BCL-2 overexpression.

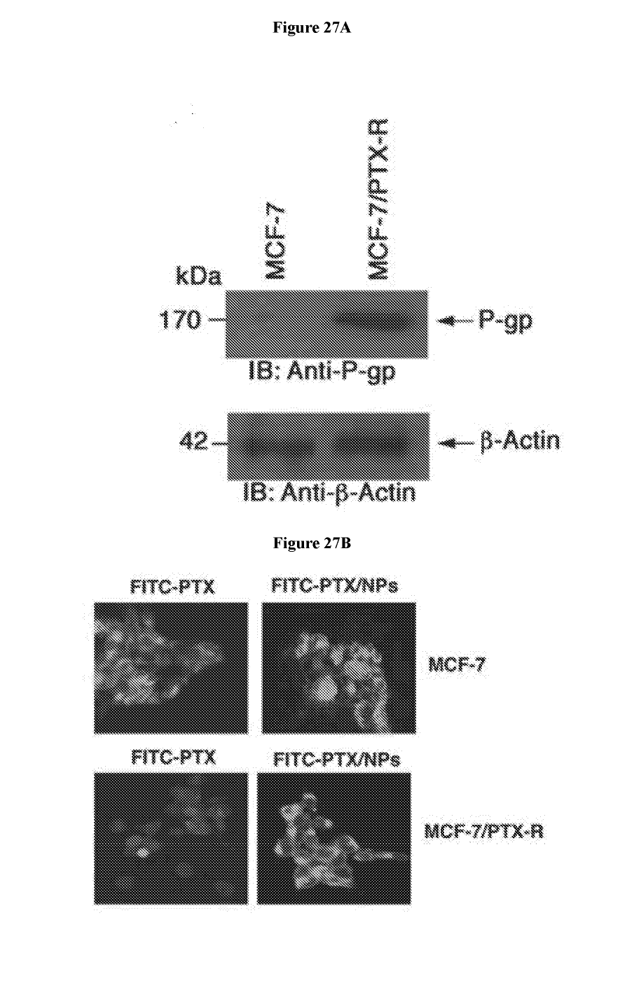

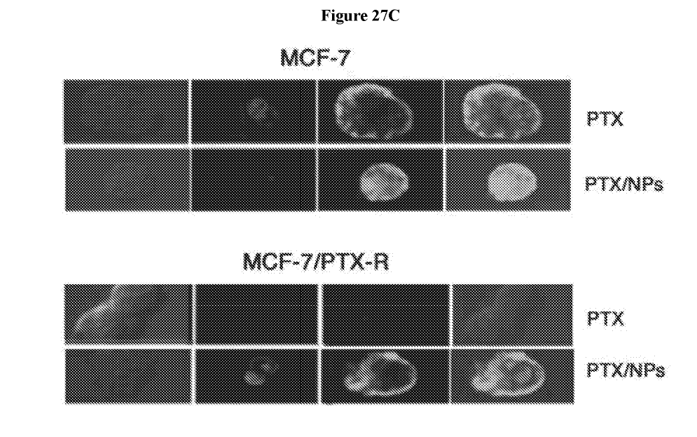

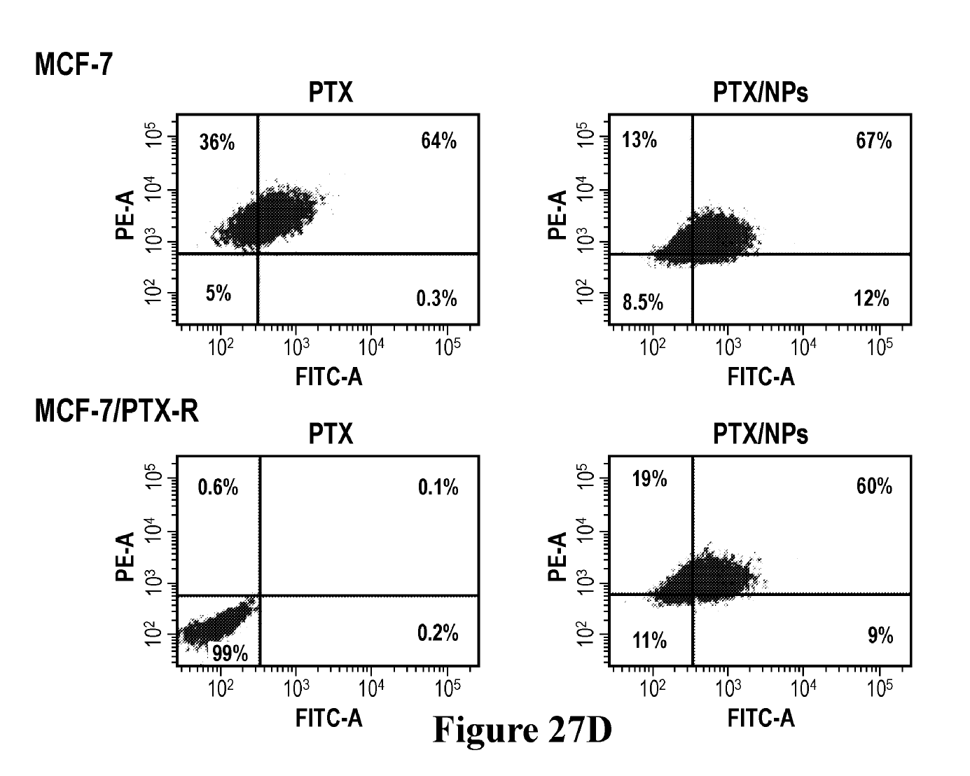

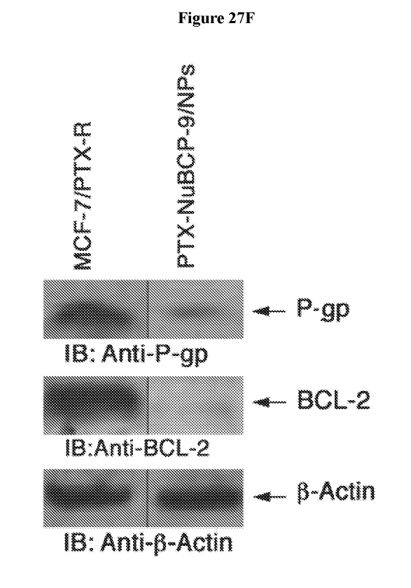

The findings that PTX-NuBCP-9/NPs are synergistic in inducing apoptosis invoked the possibility that these NPs could be effective against PTX-resistant cells. Accordingly, MCF-7 cells resistant to PTX were generated by exposure to increasing PTX concentrations (Table 9). Notably, the MCF-7/PTX-R cells were also resistant to nab-paclitaxel, but not PTX/NPs (Table 9). To define the mechanistic basis for sensitivity of MCF-7/PTX-R cells to PTX/NPs, wild-type and PTX-resistant MCF-7 cells were analyzed for P-gp expression and found that, consistent with previous reports (Brown T, et al. (1991) J. Clin. Oncol. 9(7):1261-1267; Wiernik P H, et al. (1987) Cancer Res 47(9):2486-2493; Wiernik P H, et al. (1987). J. Clin. Oncol. 5(8):1232-1239), resistance is associated with upregulation of P-gp (FIG. 27A). In concert with P-gp overexpression, intracellular FITC-PTX was markedly decreased in MCF-7/PTX-R, as compared to wild-type MCF-7, cells (FIG. 27B). Moreover and strikingly, treatment of MCF-7/PTX-R cells with FITC-PTX/NPs was associated with intracellular retention of FITC-PTX (FIG. 27B), supporting the notion that these polymeric NPs inhibit PTX efflux. Treatment of MCF-7/PTX-R cells with PTX/NPs, but not PTX or nab-paclitaxel, was also associated with induction of apoptosis as evidenced by (i) Annexin V/PI staining (FIG. 27C), (ii) quantification by FLOW analysis (FIG. 27D) and (iii) caspase-3 and PARP cleavage (FIG. 27E). The observation that P-gp and BCL-2 are upregulated in MCF-7/PTX-R cells suggested that targeting both potential mechanisms of PTX resistance may be needed to fully enhance PTX activity. Accordingly, MCF-7/PTX-R cells were treated with PTX-NuBCP-9/NPs and found an IC₅₀ of 10.3 nM, which is 5-fold lower than that obtained with PTX/NPs (Table 9). Additionally, treatment of MCF-7/PTX-R cells with PTX-NuBCP-9/NPs was associated with significant inhibition of P-gp and BCL-2 levels (FIG. 27F).

These findings provided support for a model in which PTX-NuBCP-9/NPs increase intracellular levels of PTX by blocking Pgp1 and target BCL-2 to effectively reverse PTX resistance.

### List of Tables

Table 1 provides the details of PEG-PPG-PEG block copolymer used for the preparation of the PLA-PEG-PPG-PEG copolymer

| Sl. No. | Mol. wt. | Chemical Name | Composition |
|---|---|---|---|
| 1 | 1100 | PEG-PPG-PEG 1100 | PEG 10% wt. |
| 2 | 4400 | PEG-PPG-PEG 4400 | PEG 30% wt. |
| 3 | 8400 | PEG-PPG-PEG 8400 | PEG 80% wt. |

Table 2 shows the characterization of PLA-PEG-PPG-PEG nanoparticles

| | Particle Size | | |
|---|---|---|---|
| | (nm) | | PDI |
| | (Diffraction | Zeta (ζ) Potential | (polydispersity |
| Sample | study) | (surface charge) | index) |
| PLA | 125 | −15.8 | 0.099 |
| PLA-PEG-PPG- | 120 | −1.89 | 0.11 |
| PEG(1100) | | | |
| PLA-PEG-PPG-PEG | 117 | −5.86 | 0.105 |
| (4400) | | | |
| PLA-PEG-PPG-PEG | 114 | −3.6 | 0.097 |
| (8400) | | | |

Table 3 shows the loading efficacy of the PLA-PEG-PPG-PEG nanoparticles synthesized using varying molecular weights of the polymer PEG-PPG-PEG.

| | Total Pyrene content | Loading in NP's | Percent |
|---|---|---|---|
| Nanoparticle | (mg/50 mg of PLA) | (mg/50 mg of PLA) | loading |
| PLA-PEG-PPG- | 6.21 | 6.14 | 98.84 |
| PEG (1100) | | | |
| PLA-PEG-PPG- | 2.68 | 2.33 | 96.74 |
| PEG (4400) | | | |
| PLA-PEG-PPG- | 1.74 | 1.69 | 97.29 |
| PEG (8400) | | | |

Table 4 provides the loading percent of unmodified anticancer peptide drug, NuBCP-9 in PLA-PEG-PPG-PEG nanoparticles

| | | Encapsulated | |
|---|---|---|---|
| Sample | Total Peptide (μg) | peptide (μg) | Loading % |
| | 2242.49 | 998.27 | 44.52 |
| PEG-PPG-PEG 1100 | 2242.49 | 1125.34 | 50.18 |
| PEG-PPG-PEG 4400 | 2242.49 | 1457.99 | 65.02 |
| PEG-PPG-PEG 8400 | 2242.49 | 1459.77 | 65.10 |

Table 5 provides the loading percent of modified anticancer peptide drug NuBCP-9 in PLA-PEG-PPG-PEG nanoparticles

| | | Encapsulated | |
|---|---|---|---|
| Sample | Total Peptide (μg) | peptide (μg) | % Loading |
| | 2112.23 | 1434.23 | 67.90 |
| PEG-PPG-PEG 1100 | 2112.23 | 1498.76 | 70.96 |
| PEG-PPG-PEG 4400 | 2112.23 | 1545.14 | 73.15 |
| PEG-PPG-PEG 8400 | 2112.23 | 1578.23 | 74.72 |

Table 6 provides the data obtained from proliferation studies of a MUC1 cytoplasmic domain peptide linked to a polyarginine protein transduction domain loaded in PLA-PEG-PPG-PEG nanoparticles. (* indicates a concentration of 1 mg/well)

| | | Dead | Live | Total | | Mean (% | Mean (% |
|---|---|---|---|---|---|---|---|
| No. | Formulation | Cells | Cells | Cells | % Dead | Dead) | Live) |
| 1 | NP'S* | 20000 | 125000 | 145000 | 13.79 | 14.79 | 85.21 |
| 2 | | 21000 | 112000 | 133000 | 15.79 | | |
| 3 | 20 μM | 99500 | 48000 | 147500 | 67.46 | 66.32 | 33.68 |
| 4 | MUC1-NPS | 94500 | 50500 | 145000 | 65.17 | | |
| 5 | 30 μM | 97000 | 35500 | 132500 | 73.21 | 72.22 | 27.78 |
| 6 | MUC1-NPS | 99000 | 40000 | 139000 | 71.22 | | |
| 7 | PBS | 12500 | 117500 | 130000 | 9.62 | 10.11 | 89.89 |
| 8 | (Control) | 13100 | 110500 | 123600 | 10.60 | | |

Table 7 provides the size, zeta potential, % EE or singly or dually loaded PLA72K-PEG-PPG-PEG 12.5K NPs

| | PTX:Peptide | Drug/polymer | EE % of | EE % of | | Zeta (ζ) | |
|---|---|---|---|---|---|---|---|
| Samples | (w/w) | ratio | PTX | Peptide | Size | P | PDI |
| PLA | — | — | 89.3 | 41.25 | 100 ± 5.6 | −24.1 ± 2.1 | 0.136 |
| PLA-PEG- | — | — | — | — | 104 ± 7.2 | −17.9 ± 1.3 | 0.09 |
| PPG-PEG | | | | | | | |
| Peptide-NPs | 0:1 | 1:10 | — | 64.61 | 130.1 | −28.7 | 0.10 |
| PTX-NPs | 1:0 | 1:10 | 87.63 | — | 135.4 | −3.21 | 0.12 |
| PTX-Peptide | 3:1 | 1:10 | 96.84 | 12.74 | 172 ± 4.8 | −8.57 | 0.11 |
| (F1) | | | | | | | |
| PTX-Peptide | 1:1 | 1:10 | 98.96 | 20.01 | 165.1 ± 6.4 | −9.23 | 0.01 |
| (F2) | | | | | | | |
| PTX-Peptide | 1:3 | 1:10 | 99.19 | 28.77 | 160.1 ± 9.1 | −11.3 | 0.071 |
| (F3) | | | | | | | |
| PTX-Peptide | 1:4 | 1:10 | 99.35 | 34.05 | 159.6 ± 8.6 | −24.2 | 0.096 |
| (F4) | | | | | | | |

Table 8 shows Data in connection with FIGS. 18A-F: These results demonstrate that combining PTX with L-NuBCP-9 in nanoparticles substantially reduces the effective dose of PTX by 38 fold (from 38 nM to 1 nM). The NuBCP-9 dose is also reduced from 3600 nM to 12 nM (˜300 fold reduction).

IC50 (nM)

| Samples | MCF 7 | MDA-MB 231 |
|---|---|---|
| PTX | 38 | 46 |
| PTX-NPs | 85 | 113 |
| NuBCP-9-NPs | 2000 | 3600 |
| PTX + NuBCP-9 NPs | 17 | 22 |
| PTX − NuBCP-9 NPs | 1 | 12 |

Table 9 shows IC₅₀ values of PTX, nab-paclitaxel, PTX/NPs and PTX-NuBCP-9/NPs in MCF-7 and MCF-7/PTX-R cell lines.

IC₅₀ (μM)

| Treatments | MCF-7 | MCF-7/PTX-R |
|---|---|---|
| PTX | 0.027 | 1.75 |
| nab-paclitaxel | 0.036 | 1.80 |
| PTX/NPs | 0.042 | 0.05 |
| PTX − NuBCP-9/NPs | 0.002 | 0.01 |

1. A composition comprising
a) polymeric nanoparticles comprising a poly(lactic acid)-poly(ethylene glycol)-poly(propylene glycol)-poly(ethylene glycol) (PLA-PEG-PPG-PEG) tetra block copolymer;
b) one or more chemotherapeutic agents or anti-cancer targeting agents; and
c) a peptide comprising NuBCP-9 (SEQ ID NO: 1) or a peptide comprising MUC1 (SEQ ID NO: 2).

2. (canceled)

3. (canceled)

4. The composition of claim 1, wherein the molecular weight of PLA is between about 2,000 and about 80,000 daltons.

5. The composition of claim 1, wherein the PLA-PEG-PPG-PEG tetra block copolymer is formed from chemical conjugation of PEG-PPG-PEG tri-block copolymer with PLA, and wherein the PEG-PPG-PEG tri-block copolymer can be of different molecular weights.

6. The composition of claim 1, wherein the polymeric nanoparticles are loaded with
a) a chemotherapeutic agent or a targeted anti-cancer agent; and
b) a peptide comprising NuBCP-9 (SEQ ID NO: 1) or a peptide comprising MUC1 (SEQ ID NO: 2).

7. (canceled)

8. (canceled)

9. The composition of 6, wherein the chemotherapeutic agent is paclitaxel or gemcitabine.

10. The composition of claim 9, wherein the polymeric nanoparticles are loaded with the chemotherapeutic agent and a peptide comprising NuBCP-9 (SEQ ID NO: 1) in a ratio of about 9:1, 8:2, 7:3, 6:4, 5:5, 4:6, 3:7, 2:8, or 1:9.

11. (canceled)

12. (canceled)

13. The composition of claim 6, wherein the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

14. A method of
treating a disease selected from the group consisting of cancer, an autoimmune disease, an inflammatory disease, a metabolic disorder, a developmental disorder, a cardiovascular disease, liver disease, an intestinal disease, an infectious disease, an endocrine disease and a neurological disorder in a subject in need thereof comprising administering a therapeutically effective amount of a composition of claim 1 to the subject.

15. (canceled)

16. (canceled)

17. The composition of claim 1, wherein the polymeric nanoparticles consist essentially of poly(lactic acid)-poly(ethylene glycol)-poly(propylene glycol)-poly(ethylene glycol) (PLA-PEG-PPG-PEG) tetra block copolymer.

18. The composition of claim 1, wherein the polymeric nanoparticles further comprise a targeting moiety attached to the outside of the polymeric nanoparticles, and wherein the targeting moiety is an antibody, peptide, or aptamer.

19. A polymeric nanoparticle consisting essentially of a PLA-PEG-PPG-PEG tetra block copolymer, wherein the polymeric nanoparticles are loaded with paclitaxel and a peptide comprising NuBCP-9 (SEQ ID NO: 1) or a peptide comprising MUC1 (SEQ ID NO: 2).

20. (canceled)

21. A method for treating cancer in a subject in need thereof comprising administering to the subject a therapeutically effective amount of a pharmaceutical composition comprising
a) polymeric nanoparticles comprising a PLA-PEG-PPG-PEG tetra block copolymer;
b) a chemotherapeutic agent and/or an anti-cancer targeted agent; and
c) a peptide comprising NuBCP-9 (SEQ ID NO: 1) or a peptide comprising MUC1 (SEQ ID NO: 2).

22. (canceled)

23. (canceled)

24. The method of claim 21, wherein the chemotherapeutic agent is paclitaxel or gemcitabine.

25. The method of claim 24, wherein the polymeric nanoparticles are loaded with the chemotherapeutic agent and a peptide comprising NuBCP-9 (SEQ ID NO: 1) in a ratio of about 9:1, 8:2, 7:3, 6:4, 5:5, 4:6, 3:7, 2:8, or 1:9.

26. (canceled)

27. (canceled)

28. The method of claim 21, wherein the chemotherapeutic agent or targeted anti-cancer agent is selected from the group consisting of doxorubicin, daunorubicin, decitabine, irinotecan, SN-38, cytarabine, docetaxel, triptolide, geldanamycin, 17-AAG, 5-FU, oxaliplatin, carboplatin, taxotere, methotrexate, and bortezomib.

29. The method of claim 21, wherein the cancer is breast cancer, prostate cancer, non-small cell lung cancer, metastatic colon cancer, pancreatic cancer, or a hematological malignancy.

30. The method of claim 21, wherein the subject is resistant to treatment with paclitaxel or nab-paclitaxel.

31. The method of claim 21, wherein the subject is refractory to treatment with paclitaxel or nab-paclitaxel.

32. The method of claim 21, wherein the subject is in relapse after treatment with paclitaxel or nab-paclitaxel.

33. A method for inhibiting paclitaxel efflux in a cell comprising contacting the cell with an effective amount of polymeric nanoparticles comprising PLA-PEG-PPG-PEG tetra block copolymer.

34. (canceled)

35. A method for blocking P-glycoprotein expression in a cell comprising contacting the cell with an effective amount of polymeric nanoparticles comprising PLA-PEG-PPG-PEG tetra block copolymer.

36. A method for reversing P-glycoprotein-mediated drug resistance in a cell comprising contacting the cell with an effective amount of polymeric nanoparticles comprising PLA-PEG-PPG-PEG tetra block copolymer.

37. The method of claim 21, wherein the polymeric nanoparticles consist essentially of PLA-PEG-PPG-PEG tetra block copolymer.

38. A method for causing apoptosis in a cancer cell having resistance against a first chemotherapeutic comprising contacting the cancer cell with polymeric nanoparticles comprising PLA-PEG-PPG-PEG tetra block copolymer, wherein the polymeric nanoparticles are loaded with a second chemotherapeutic, and wherein the resistance of the cancer cell against the first chemotherapeutic is caused by upregulation of P-glycoprotein.

39. (canceled)

40. (canceled)

41. (canceled)

42. (canceled)

43. (canceled)

44. (canceled)


SEQUENCE LISTING (5 sequences)

SEQ ID NO: 1 - protein, 9 aa, Homo sapiens
FSRSLHSLL

SEQ ID NO: 2 - protein, 7 aa, Homo sapiens
CQCRRKN

SEQ ID NO: 3 - protein, 16 aa, Homo sapiens
RRRRRRRRRCQCRRKN

SEQ ID NO: 4 - protein, 7 aa, Homo sapiens
AQARRKN

SEQ ID NO: 5 - protein, 32 aa, Homo sapiens
RRRRRRRRRAQARRKNRRRRRRRRRAQARRKN